(19) United States
(12) Patent Application Publication
(10) Pub. No.: US 2008/0004269 A1
(43) Pub. Date: Jan. 3, 2008

(54) Pyrazolylmethy Heteroaryl Derivatives

(76) Inventors: Yuelian Xu, East Haven, CT (US); Linghong Xie, Guilford, CT (US); Yang Gao, Madison, CT (US); Bingsong Han, North Haven, CT (US); George D. Maynard, Clinton, CT (US); Bertrand L. Chenard, Waterford, CT (US); Jiong Lan, Moraga, CA (US)

Correspondence Address: NEUROGEN CORPORATION, 35 NORTHEAST INDUSTRIAL ROAD, BRANFORD, CT, 06405, US

(21) Appl. No.: 11/718,528

(22) PCT Filed: Nov. 1, 2005
(86) PCT No.: PCT/US05/39488

§ 371 Date: May 3, 2007

Related U.S. Application Data
(60) Provisional application No. 60/625,313, filed on Nov. 4, 2004.

(51) Int. Cl. A61K 31/506; A61K 31/444; A61K 31/519; A61P 25/22; A61P 25/28; C07D 403/14; C07D 487/04; C07D 413/14; C07D 239/34; A61P 25/24; A61K 31/5377; A61K 31/497

(52) U.S. Cl. 514/233.2; 514/255.05; 514/256; 514/259.31; 514/333; 544/118; 544/263; 544/319; 544/328; 544/333; 544/405; 546/256

(57) ABSTRACT

Compounds of Formula (I) are provided, as are methods for their preparation. The variables W, X, Y, Z, R₅, R₈ and Ar in the above formula are defined herein. Such compounds may be used to modulate ligand binding to GABA_{A} receptorsin vivo or in vitro, and are particularly useful in the treatment of a variety of central nervous system (CNS) disorders in humans, domesticated companion animals and livestock animals. Compounds provided herein may be administered alone or in combination with one or more other CNS agents to potentiate the effects of the other CNS agent(s). Pharmaceutical compositions and methods for treating such disorders are provided, as are methods for using such ligands for detecting GABA_{A} receptors (e.g., receptor localization studies).

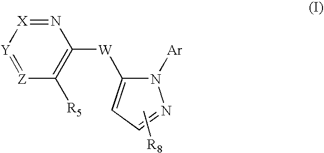

### FIELD OF THE INVENTION

The present invention relates generally to pyrazolylmethyl heteroaryl derivatives that have useful pharmacological properties. The invention further relates to pharmaceutical compositions comprising such compounds and to the use of such compounds in the treatment of central nervous system (CNS) disorders.

### BACKGROUND OF THE INVENTION

The GABA_{A} receptor superfamily represents one of the classes of receptors through which the major inhibitory neurotransmitter γ-aminobutyric acid (GABA) acts. Widely, although unequally, distributed throughout the mammalian brain, GABA mediates many of its actions through interaction with a complex of proteins called the GABA_{A} receptor, which causes alteration in chloride conductance and membrane polarization. A number of drugs, including the anxiolytic and sedating benzodiazepines, also bind to this receptor. The GABA_{A} receptor comprises a chloride channel that opens in response to GABA, allowing chloride to enter the cell. This, in turn, effects a slowing of neuronal activity through hyperpolarization of the cell membrane potential.

GABA_{A} receptors are composed of five protein subunits. A number of cDNAs for these GABA_{A} receptor subunits have been cloned and their primary structures determined. While these subunits share a basic motif of 4 membrane-spanning helices, there is sufficient sequence diversity to classify them into several groups. To date, at least six α, three β, three γ, one ε, one δ and two ρ subunits have been identified. Native GABA_{A} receptors are typically composed of two α subunits, two β subunits and one γ subunit. Various lines of evidence (such as message distribution, genome localization and biochemical study results) suggest that the major naturally occurring receptor combinations are β₁β₂γ₂, α₂β₃γ₂, α₃ β₃γ₂ and α₅β₃γ₂.

The GABA_{A} receptor binding sites for GABA (two per receptor complex) are formed by amino acids from the α and β subunits. Amino acids from the α and γ subunits together form one benzodiazepine site per receptor, at which benzodiazepines exert their pharmacological activity. In addition, the GABA_{A} receptor contains sites of interaction for several other classes of drugs. These include a steroid binding site, a picrotoxin site and a barbiturate site. The benzodiazepine site of the GABA_{A} receptor is a distinct site on the receptor complex that does not overlap with the sites of interaction for other classes of drugs or GABA.

In a classic allosteric mechanism, the binding of a drug to the benzodiazepine site alters the affinity of the GABA receptor for GABA. Benzodiazepines and related drugs that enhance the ability of GABA to open GABA_{A} receptor channels are known as agonists or partial agonists, depending on the level of GABA enhancement. Other classes of drugs, such as β-carboline derivatives, that occupy the same site and negatively modulate the action of GABA are called inverse agonists. Those compounds that occupy the same site, and yet have little or no effect on GABA activity, can block the action of agonists or inverse agonists and are thus referred to as GABA_{A} receptor antagonists.

The important allosteric modulatory effects of drugs acting at the benzodiazepine site were recognized early, and the distribution of activities at different receptor subtypes has been an area of intense pharmacological discovery. Agonists that act at the benzodiazepine site are known to exhibit anxiolytic, sedative, anticonvulsant and hypnotic effects, while compounds that act as inverse agonists at this site elicit anxiogenic, cognition enhancing and proconvulsant effects.

While benzodiazepines have enjoyed long pharmaceutical use, these compounds can exhibit a number of unwanted side effects. Accordingly, there is a need in the art for additional therapeutic agents that modulate GABA_{A} receptor activation and/or activity. The present invention fulfills this need, and provides further related advantages.

### SUMMARY OF THE INVENTION

The present invention provides compounds of Formula I: 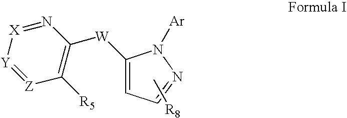
as well as pharmaceutically acceptable salts thereof, wherein:
W is CR₆R₇ or O;
X is nitrogen (optionally taken together with R₂ to form a fused 5-membered heteroaryl), NO or CR₁;
Y is nitrogen (optionally taken together with R₃ to form a fused 5-membered heteroaryl), NO or CR₂;
Z is nitrogen (optionally taken together with R₂ to form a fused 5-membered heteroaryl), NO or CR₃;
such that no more than two of X, Y and Z are nitrogen or NO;
R₁ is chosen from R_{C};
With respect to R₂ and R₃:
   (i) R₂ and R₃ are independently chosen from R_{C}; or
   (ii) Z is nitrogen and R₂ is taken together with Z to form a fused, 5-membered heteroaryl that contains 2 or 3 ring nitrogen atoms, with remaining ring atoms being carbon, and that is substituted with from 0 to 3 substituents independently chosen from R₄; or
   (iii) X is nitrogen and R₂ is taken together with X to form a fused, 5-membered heteroaryl that contains 1, 2 or 3 ring nitrogen atoms, with remaining ring atoms being carbon, and that is substituted with from 0 to 3 substituents independently chosen from R₄; and R₃ is chosen from R_{C}; or
   (iv) Y is nitrogen and R₃ is taken together with Y to form a fused 5-membered heteroaryl that contains 2 or 3 ring nitrogen atoms, with remaining ring atoms being carbon, and that is substituted with from 0 to 3 substituents independently chosen from R₄;
Each R₄ is independently chosen from R_{C};
R₅ is:
   (a) hydrogen, halogen or cyano; or
   (b) C₁-C₆alkyl, C₂-C₆alkenyl, C₂-C₆alkynyl, C₁-C₄alkoxy, or mono- or di-(C₁-C₄alkyl)amino, each of which is optionally substituted, and is preferably substituted with from 0 to 5 substituents independently chosen from halogen, hydroxy, nitro, cyano, amino, C₁-C₄alkoxy, C₁-C₂haloalkyl, C₁-C₂haloalkoxy, mono- and di-(C₁-C₄alkyl)amino, C₃-C₈cycloalkyl, phenyl, phenylC₁-C₄alkoxy and 5- or 6-membered heteroaryl;
   such that if W is O, then R₅ is not hydrogen;
R₆, and R₇ are independently hydrogen, methyl, ethyl or halogen;
R₈ represents 0, 1 or 2 substituents independently chosen from halogen, hydroxy, nitro, cyano, amino, C₁-C₄alkyl, C₁-C₄alkoxy, mono- and di(C₁-C₄alkyl)amino, C₃-C₇cycloalkyl, C₁-C₂haloalkyl and C₁-C₂haloalkoxy;
Each R_{C} is independently chosen from:
   (a) hydrogen, halogen, nitro and cyano; and
   (b) groups of the formula: 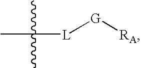
   wherein:
      L is absent, a single covalent bond or C₁-C₈alkylene;
      G is a single covalent bond, N(R_{B}) (i.e., 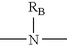
      ), O, C(═O) (i.e., 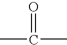
      ), C(═O)O (i.e., 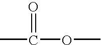
      ),
      C(═O)N(R_{B}) (i.e., 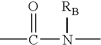
      ), N(R_{B})C(═O) (i.e., 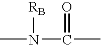
      ), S(O)ₘ (i.e., —S—, 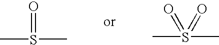
      ), CH₂C(═O) (i.e., 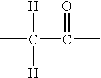
      ), S(O)ₘN(R_{B}) (e.g., 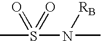
      ) or N(R_{B})S(O)ₘ
      (e.g., 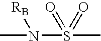
      ); wherein m is 0, 1 or 2; and
R_{A} and each R_{B} are independently selected from:
   (i) hydrogen; and
   (ii) C₁-C₈alkyl, C₂-C₈alkenyl, C₂-C₈alkynyl, (C₃-C₈cycloalkyl)C₀-C₄alkyl, (3- to 7-membered heterocycloalkyl)C₀-C₄alkyl, (C₆-C₁₀aryl)C₀-C₂alkyl and (5- to 10-membered heteroaryl)C₀-C₂alkyl, each of which is optionally substituted, and is preferably substituted with from 0 to 4 substituents independently selected from halogen, hydroxy, nitro, cyano, amino, oxo, C₁-C₄alkyl, C₁-C₄alkoxy, C₁-C₄alkanoyl, mono- and di(C₁-C₄alkyl)amino, C₁-C₄haloalkyl and C₁-C₄haloalkoxy; and
Ar represents phenyl, naphthyl or 5- to 10-membered heteroaryl, each of which is optionally substituted, and is preferably substituted with from 0 to 4 substituents independently chosen from halogen, hydroxy, nitro, cyano, amino, aminocarbonyl, C₁-C₈alkyl, C₁-C₈alkenyl, C₁-C₈alkynyl, C₁-C₈alkoxy, (C₃-C₇cycloalkyl)C₀-C₄alkyl, (C₃-C₇cycloalkyl)C₁-C₄alkoxy, C₁-C₈alkyl ether, C₁-C₈alkanone, C₁-C₈alkanoyl, (3- to 7-membered heterocycle)C₀-C₄alkyl, C₁-C₈haloalkyl, C₁-C₈haloalkoxy, oxo, C₁-C₈hydroxyalkyl, C₁-C₈-aminoalkyl, and mono- and di-(C₁-C₈alkyl)aminoC₀-C₈alkyl.

Within certain aspects, such compounds are GABA_{A} receptor modulators provided herein that modulate GABA_{A} receptor activation and/or GABA_{A} receptor-mediated signal transduction. Such GABA_{A} receptor modulators are preferably high affinity and/or high selectivity GABA_{A} receptor ligands and act as agonists, inverse agonists or antagonists of GABA_{A} receptors, such as human GABA_{A} receptors. As such, they are useful in the treatment of various CNS disorders.

Within further aspects, the present invention provides pharmaceutical compositions comprising one or more compounds or salts as described above in combination with a pharmaceutically acceptable carrier, diluent or excipient. Packaged pharmaceutical preparations are also provided, comprising such a pharmaceutical composition in a container and instructions for using the composition to treat a patient suffering from a CNS disorder such as anxiety, depression, a sleep disorder, sleepwalking, attention deficit disorder, schizophrenia, or a cognitive disorder such as short-term memory loss or Alzheimer's dementia.

The present invention further provides, within other aspects, methods for treating patients suffering from certain CNS disorders, such as anxiety, depression, a sleep disorder, sleepwalking, attention deficit disorder, schizophrenia or a cognitive disorder, comprising administering to a patient in need of such treatment a therapeutically effective amount of a compound or salt of Formula I. Methods for improving short term memory in a patient are also provided, comprising administering to a patient in need of such treatment a therapeutically effective amount of a compound or salt of Formula I. Treatment of humans, domesticated companion animals (pets) or livestock animals suffering from certain CNS disorders with a compound or salt of Formula I is encompassed by the present invention.

In a separate aspect, the invention provides methods of potentiating the action of other CNS active compounds. These methods comprise administering to a patient a therapeutically effective amount of a compound or salt of Formula I in conjunction with the administration of a therapeutically effective amount of a different CNS active compound.

The present invention further relates to the use of compounds and salts of Formula I as probes for the localization of GABA_{A} receptors in sample (e.g., a tissue section). In certain embodiments, GABA_{A} receptors are detected using autoradiography. Additionally, the present invention provides methods for determining the presence or absence of GABA_{A} receptor in a sample, comprising the steps of: (a) contacting a sample with a compound or salt of Formula I under conditions that permit binding of the compound to GABA_{A} receptor; (b) removing compound or salt that is not bound to the GABA_{A} receptor and (c) detecting compound or salt bound to GABA_{A} receptor.

In yet another aspect, the present invention provides methods for preparing the compounds disclosed herein, including the intermediates.

These and other aspects of the present invention will become apparent upon reference to the following detailed description.

### DETAILED DESCRIPTION OF THE INVENTION

As noted above, the present invention provides compounds and salts of Formula I. Certain preferred compounds bind to GABA_{A} receptor, preferably with high selectivity; more preferably such compounds further provide beneficial modulation of brain function. Without wishing to be bound to any particular theory of operation, it is believed that that interaction of such compounds with the benzodiazepine site of GABA_{A} receptor results in the pharmacological effects of these compounds. Such compounds may be used in vitro or in vivo to determine the location of GABA_{A} receptors or to modulate GABA_{A} receptor activity in a variety of contexts.

Chemical Description and Terminology

Compounds provided herein are generally described using standard nomenclature. For compounds having asymmetric centers, it should be understood that (unless otherwise specified) all of the optical isomers and mixtures thereof are encompassed. All chiral (enantiomeric and diastereomeric) and racemic forms, as well as all geometric isomeric forms of a structure are intended, unless the specific stereochemistry or isomeric form is specifically indicated. Geometric isomers of olefins, C═N double bonds and the like may also be present in the compounds described herein, and all such stable isomers are contemplated in the present invention. Cis and trans geometric isomers are also contemplated and may be isolated as a mixture of isomers or as separated isomeric forms. Compounds in which one or more atoms are replaced with an isotope (i.e., an atom having the same atomic number but a different mass number) are also contemplated. By way of general example, and without limitation, isotopes of hydrogen include tritium and deuterium, and isotopes of carbon include ¹¹C, ¹³C and ¹⁴C.

Certain general formulas recited herein includes variables. Unless otherwise specified, each variable within such a formula is defined independently of other variables, and any variable that occurs more than one time within a formula is defined independently at each occurrence. Thus, for example, if a group is described as being substituted with 0-2 R*, then the group may be unsubstituted or substituted with up to two R* groups and R* at each occurrence is selected independently from the definition of R*. In addition, it will be apparent that combinations of substituents and/or variables are permissible only if such combinations result in a stable compound (i.e., a compound that can be isolated, characterized and tested for biological activity).

A “pharmaceutically acceptable salt” is an acid or base salt form of a compound, which salt form is suitable for use in contact with the tissues of human beings or animals without excessive toxicity or carcinogenicity, and preferably without irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, EtOAc, EtOH, isopropanol or acetonitrile, is preferred.

It will be apparent that each compound of Formula I may, but need not, be formulated as a hydrate, solvate or non-covalent complex. In addition, the various crystal forms and polymorphs are within the scope of the present invention. Also provided herein are prodrugs of the compounds of Formula I. A “prodrug” is a compound that may not fully satisfy the structural requirements of the compounds provided herein, but is modified in vivo, following administration to a patient, to produce a compound of Formula I, or other formula provided herein. Prodrugs include compounds wherein hydroxy, amine or sulfhydryl groups are bonded to any group that, when administered to a mammalian subject, cleaves to form a free hydroxy, amino or sulfhydryl group, respectively. For example, a prodrug may be an acylated derivative of a compound of Formula I. Further examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved in vivo to yield the parent compounds.

A “substituent,” as used herein, refers to a molecular moiety that is covalently bonded to an atom within a molecule of interest. For example, a “ring substituent” may be a moiety such as a halogen, alkyl group, haloalkyl group or other substituent discussed herein that is covalently bonded to an atom (preferably a carbon or nitrogen atom) that is a ring member. The term “substitution” refers to replacing a hydrogen atom in a molecular structure with a substituent as described above, such that the valence on the designated atom is not exceeded, and such that a chemically stable compound (i.e., a compound that can be isolated, characterized, and tested for biological activity) results from the substitution. When a substituent is oxo (i.e., ═O), then 2 hydrogens on the atom are replaced. When aromatic moieties are substituted with an oxo group, the aromatic ring is replaced by the corresponding partially unsaturated ring. For example a pyridyl group substituted with oxo is a pyridone.

The phrase “optionally substituted” indicates that a group may either be unsubstituted or substituted at one or more of any of the available positions, typically at 1, 2, 3, 4 or 5 positions, by one or more suitable substituents such as those disclosed herein. Optional substitution is also indicated by the phrase “substituted with from 0 to X substituents,” in which X is the maximum number of substituents.

A dash (“-”) that is not between two letters or symbols is used to indicate a point of attachment for a substituent. For example, the amide substituent —C(═O)NH₂ is attached via the carbon atom.

As used herein, “alkyl” is intended to include both branched and straight-chain saturated aliphatic hydrocarbon groups; where specified, such a group has the indicated number of carbon atoms. Thus, the term C₁-C₆alkyl, as used herein, indicates an alkyl group having from 1 to 6 carbon atoms. “C₀-C₄alkyl” refers to a single covalent bond or a C₁-C₄alkyl group. Alkyl groups include groups having from 1 to 8 carbon atoms (C₁-C₈alkyl), from 1 to 6 carbon atoms (C₁-C₆alkyl) and from 1 to 4 carbon atoms (C₁-C₄alkyl), such as methyl, ethyl, n-propyl, isopropyl, n-butyl, sec-butyl, tert-butyl, pentyl, 2-pentyl, isopentyl, neopentyl, hexyl, 2-hexyl, 3-hexyl and 3-methylpentyl. In certain embodiments, preferred alkyl groups are methyl, ethyl, propyl, butyl and 3-pentyl. “Aminoalkyl” is an alkyl group as defined herein substituted with one or more —NH₂ substituents. “Hydroxyalkyl” is an alkyl group as defined herein substituted with one or more —OH substituents.

“Alkylene” refers to a divalent alkyl group, as defined above. C₀-C₃alkylene is a single covalent bond or an alkylene group having 1, 2 or 3 carbon atoms.

“Alkenyl” refers to a straight or branched hydrocarbon chain comprising one or more carbon-carbon double bonds, such as ethenyl and propenyl. Alkenyl groups include C₂-C₈alkenyl, C₂-C₆alkenyl and C₂-C₄alkenyl groups (which have from 2 to 8, 2 to 6 or 2 to 4 carbon atoms, respectively), such as ethenyl, allyl or isopropenyl.

“Alkynyl” refers to straight or branched hydrocarbon chains comprising one or more carbon-carbon triple bonds. Alkynyl groups include C₂-C₈alkynyl, C₂-C₆alkynyl and C₂-C₄alkynyl groups, which have from 2 to 8, 2 to 6 or 2 to 4 carbon atoms, respectively. Alkynyl groups include, for example, groups such as ethynyl and propynyl.

By “alkoxy,” as used herein, is meant an alkyl group as described above attached via an oxygen bridge. Alkoxy groups include C₁-C₆alkoxy and C₁-C₄alkoxy groups, which have from 1 to 6 or 1 to 4 carbon atoms, respectively. Methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy and 3-methylpentoxy are specific alkoxy groups. Similarly “alkylthio” refers to an alkyl group attached via a sulfur bridge.

A “cycloalkyl” is a saturated or partially saturated cyclic group in which all ring members are carbon, such as cyclopropyl, cyclobutyl, cyclopentyl, cyclohexyl, cycloheptyl, cyclooctyl, norbornyl, adamantyl, decahydro-naphthalenyl, octahydro-indenyl, and partially saturated variants of any of the foregoing, such as cyclohexenyl. Cycloalkyl groups typically contain from 3 to about 10 ring carbon atoms; in certain embodiments, such groups have from 3 to 7 ring carbon atoms (i.e., C₃-C₇cycloalkyl). If substituted, any ring carbon atom may be bonded to any indicated substituent.

In the term “(cycloalkyl)alkyl,” “cycloalkyl” and “alkyl” are as defined above, and the point of attachment is on the alkyl group. Certain such groups are (C₃-C₈cycloalkyl)C₀-C₄alkyl and (C₃-C₇cycloalkyl)C₀-C₄alkyl, in which the cycloalkyl group of the indicated ring size is linked via a single covalent bond or a C₁-C₄alkylene group. This term encompasses, for example, cyclopropylmethyl, cyclohexylmethyl and cyclohexylethyl. Similarly, “(C₃-C₇cycloalkyl)C₁-C₄alkoxy” refers to a C₃-C₇cycloalkyl group linked via a C₁-C₄alkoxy, in which the oxygen atom is the point of attachment (i.e., (C₃-C₇cycloalkyl)C₁-C₄alkyl-O—).

The term “alkanoyl” refers to an alkyl group as defined above attached via a carbonyl bridge (i.e., —(C═O)-alkyl). Alkanoyl groups include C₂-C₈alkanoyl, C₂-C₆alkanoyl and C₂-C₄alkanoyl groups, which have from 2 to 8, 2 to 6 or 2 to 4 carbon atoms, respectively. “C₁alkanoyl” refers to —(C═O)—H, which (along with C₂-C₈alkanoyl) is encompassed by the term “C₁-C₈alkanoyl.” Ethanoyl is C₂alkanoyl.

The term “oxo,” as used herein, refers to a keto (C═O) group. An oxo group that is a substituent of a nonaromatic ring results in a conversion of —CH₂— to —C(═O)—. It will be apparent that the introduction of an oxo substituent on an aromatic ring destroys the aromaticity.

An “alkanone” is a ketone group in which carbon atoms are in a linear or branched alkyl arrangement. “C₃-C₈alkanone,” “C₃-C₆alkanone” and “C₃-C₄alkanone” refer to an alkanone having from 3 to 8, 6 or 4 carbon atoms, respectively. By way of example, a C₃ alkanone substituent has the structure —CH₂—(C═O)—CH₃.

Similarly, “alkyl ether” refers to a linear or branched ether substituent linked via a carbon-carbon bond. Alkyl ether groups include C₂-C₈allyl ether, C₂-C₆alkyl ether and C₂-C₄alkyl ether groups, which have 2 to 8, 6 or 4 carbon atoms, respectively. By way of example, a C₂alkyl ether group has the structure —CH₂—O—CH₃.

The term “alkoxycarbonyl” refers to an alkoxy group linked via a carbonyl (i.e., a group having the general structure —C(═O)—O-alkyl). Alkoxycarbonyl groups include C₁-C₈, C₁-C₆ and C₁-C₄alkoxycarbonyl groups, which have from 1 to 8, 6 or 4 carbon atoms, respectively, in the alkyl portion of the group. For example, “C₁alkoxycarbonyl” refers to —C(═O)—O—CH₃.

The term “aminocarbonyl” refers to an amide group (i.e., —(C═O)NH₂). “Mono- or di-(C₁-C₆alkyl)aminocarbonyl” is an aminocarbonyl group in which one or both of the hydrogen atoms is replaced with C₁-C₆alkyl. If both hydrogen atoms are so replaced, the C₁-C₆alkyl groups may be the same or different.

“Alkylamino” refers to a secondary or tertiary amine substituent having the general structure —NH-alkyl or —N(alkyl)(alkyl), wherein each alkyl may be the same or different. Such groups include, for example, mono- and di-(C₁-C₆alkyl)amino groups (in which each alkyl may be the same or different and may contain from 1 to 6 carbon atoms), as well as mono- and di-(C₁-C₄alkyl)amino groups and mono- and di-(C₁-C₂alkyl)amino groups.

“Alkylaminoalkyl” refers to an alkylamino group linked via an alkyl group (i.e., a group having the general structure -alkyl-NH-alkyl or -alkyl-N(alkyl)(alkyl)). Such groups include, for example, mono- and di-(C₁-C₈alkyl)aminoC₁-C₈alkyl, in which each alkyl may be the same or different. “Mono- or di-(C₁-C₈alkyl)aminoC₀-C₈alkyl” refers to a mono- or di-(C₁-C₈alkyl)amino group linked via a single covalent bond or a C₁-C₈alkylene group. The following are representative alkylaminoalkyl groups:

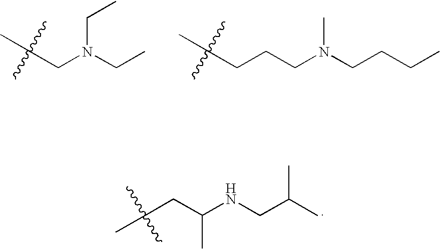

The term “halogen” refers to fluorine, chlorine, bromine and iodine.

A “haloalkyl” is a branched or straight-chain alkyl group, substituted with 1 or more halogen atoms (e.g., “C₁-C₈haloalkyl” groups have from 1 to 8 carbon atoms; “C₁-C₂haloalkyl” groups have from 1 to 2 carbon atoms). Examples of haloalkyl groups include, but are not limited to, mono-, di- or tri-fluoromethyl; mono-, di- or tri-chloromethyl; mono-, di-, tri-, tetra- or penta-fluoroethyl; and mono-, di-, tri-, tetra- or penta-chloroethyl. Typical haloalkyl groups are trifluoromethyl and difluoromethyl. The term “haloalkoxy” refers to a haloalkyl group as defined above attached via an oxygen bridge. “C₁-C₈haloalkoxy” groups have from 1 to 8 carbon atoms.

As used herein, the term “aryl” indicates aromatic groups containing only carbon in the aromatic ring(s). Such aromatic groups may be further substituted with carbon or non-carbon atoms or groups. Typical aryl groups contain 1 to 3 separate, fused, spiro or pendant rings and from 6 to about 18 ring atoms, without heteroatoms as ring members. Preferred aryl groups are 6- to 12-membered groups and 6- to 10-membered groups, such as phenyl, naphthyl (including 1-naphthyl and 2-naphthyl) and biphenyl. Arylalkyl groups are aryl groups linked via an alkylene. Such groups include, for example, (C₆-C₁₀aryl)C₀-C₂alkyl groups, which are 6- to 10-membered groups liked via a single covalent bond or a methylene or ethylene moiety. Arylalkoxy groups are aryl groups linked via an alkoxy moiety. For example, phenylC₁-C₂alkoxy refers to benzyloxy or phenylethoxy (also known as phenethyloxy).

The term “heterocycle” or “heterocyclic group” is used to indicate saturated, partially unsaturated or aromatic groups having 1 or 2 rings, with 3 to 8 atoms in each ring, and in at least one ring from 1 to 4 heteroatoms independently chosen from oxygen, sulfur and nitrogen. The heterocyclic ring may be attached via any ring heteroatom or carbon atom that results in a stable structure, and may be substituted on carbon and/or nitrogen atom(s) if the resulting compound is stable. Any nitrogen and/or sulfur heteroatoms may optionally be oxidized, and any nitrogen may optionally be quaternized.

Certain heterocycles are “heteroaryl” (i.e., comprise at least one aromatic ring having from 1 to 4 heteroatoms, with the remaining ring atoms being carbon). When the total number of S and O atoms in the heteroaryl group exceeds 1, then these heteroatoms are not adjacent to one another; preferably the total number of S and O atoms in the heteroaryl group is not more than 1, 2 or 3, more preferably not more than 1 or 2 and most preferably not more than 1. Examples of heteroaryl groups include pyridyl, indolyl, pyrimidinyl, pyridazinyl, pyrazinyl, imidazolyl, oxazolyl, thienyl, thiazolyl, triazolyl, isoxazolyl, quinolinyl, pyrrolyl, pyrazolyl and 5,6,7,8-tetrahydroisoquinoline. Bicyclic heteroaryl groups may, but need not, contain a saturated ring in addition to the aromatic ring (e.g., tetrahydroquinolinyl or tetrahydroisoquinolinyl). A “5- or 6-membered heteroaryl” is a monocyclic heteroaryl having 5 or 6 ring members.

Other heterocycles are referred to herein as “heterocycloalkyl” (i.e., saturated or partially saturated heterocycles, that do not contain a heteroaryl group). Heterocycloalkyl groups generally have from 3 to about 8 ring atoms, and more typically from 3 to 7 (or from 5 to 7) ring atoms. Examples of heterocycloalkyl groups include morpholinyl, thiomorpholinyl, piperazinyl, piperadinyl and pyrrolidinyl.

A (3- to 7-membered heterocycle)C₀-C₄alkyl is a heterocycle having from 3 to 7 ring members that is linked via a single covalent bond or a C₁-C₄alkylene group. A (3- to 7-membered heterocycloalkyl)C₀-C₄alkyl group is a heterocycloalkyl group having from 3 to 7 ring members that is linked via a single covalent bond or a C₁-C₄alkylene group. A (5- to 10-membered heterocycloalkyl)C₀-C₂alkyl group is a heteroaryl group having from 5 to 10 ring members that is linked via a single covalent bond or a methylene or ethylene group.

The terms “GABA_{A} receptor” and “benzodiazepine receptor” refer to a protein complex that detectably binds GABA and mediates a dose dependent alteration in chloride conductance and membrane polarization. Receptors comprising naturally-occurring mammalian (especially human or rat) GABA_{A} receptor subunits are generally preferred, although subunits may be modified provided that any modifications do not substantially inhibit the receptor's ability to bind GABA (i.e., at least 50% of the binding affinity of the receptor for GABA is retained). The binding affinity of a candidate GABA_{A} receptor for GABA may be evaluated using a standard ligand binding assay as provided herein. It will be apparent that there are a variety of GABA_{A} receptor subtypes that fall within the scope of the term “GABA_{A} receptor.” These subtypes include, but are not limited to, α₂62 _{3 g2}, α₃β_{3 g2}, α₅β_{3 g2} and α₁β_{2 g2} receptor subtypes. GABA_{A} receptors may be obtained from a variety of sources, such as from preparations of rat cortex or from cells expressing cloned human GABA_{A} receptors. Particular subtypes may be readily prepared using standard techniques (e.g., by introducing mRNA encoding the desired subunits into a host cell, as described herein).

An “agonist” of a GABA_{A} receptor is a compound that enhances the activity of GABA at the GABA_{A} receptor. Agonists may, but need not, also enhance the binding of GABA to GABA_{A} receptor. The ability of a compound to act as a GABA_{A} agonist may be determined using an electrophysiological assay, such as the assay provided in Example 9.

An “inverse agonist” of a GABA_{A} receptor is a compound that reduces the activity of GABA at the GABA_{A} receptor. Inverse agonists, but need not, may also inhibit binding of GABA to the GABA_{A} receptor. The reduction of GABA-induced GABA_{A} receptor activity may be determined from an electrophysiological assay such as the assay of Example 9.

An “antagonist” of a GABA_{A} receptor, as used herein, is a compound that occupies the benzodiazepine site of the GABA_{A} receptor, but has no detectable effect on GABA activity at the GABA_{A} receptor. Such compounds can inhibit the action of agonists or inverse agonists. GABA_{A} receptor antagonist activity may be determined using a combination of a suitable GABA_{A} receptor binding assay, such as the assay provided in Example 8, and a suitable functional assay, such as the electrophysiological assay provided in Example 9, herein.

A “GABA_{A} receptor modulator” is any compound that acts as a GABA_{A} receptor agonist, inverse agonist or antagonist. In certain embodiments, such a modulator may exhibit an affinity constant (Kᵢ) of less than 1 micromolar in a standard GABA_{A} receptor radioligand binding assay, or an EC₅₀ of less than 1 micromolar in an electrophysiological assay. In other embodiments a GABA_{A} receptor modulator may exhibit an affinity constant or EC₅₀ of less than 500 nM, 200 nM, 100 nM, 50 nM, 25 nM, 10 nM or 5 nM.

A GABA_{A} receptor modulator is said to have “high affinity” if the Kᵢ at a GABA_{A} receptor is less than 1 micromolar, preferably less than 100 nanomolar or less than 10 nanomolar. A representative assay for determining Kᵢ at GABA_{A} receptor is provided in Example 8, herein. It will be apparent that the Kᵢ may depend upon the receptor subtype used in the assay. In other words, a high affinity compound may be “subtype-specific” (i.e., the Kᵢ is at least 10-fold greater for one subtype than for another subtype). Such compounds are said to have high affinity for GABA_{A} receptor if the Kᵢ for at least one GABA_{A} receptor subtype meets any of the above criteria.

A GABA_{A} receptor modulator is said to have “high selectivity” if it binds to at least one subtype of GABA_{A} receptor with a Kᵢ that is at least 10-fold lower, preferably at least 100-fold lower, than the Kᵢ for binding to other (i.e., not GABA_{A}) membrane-bound receptors. In particular, a compound that displays high selectivity should have a Kᵢ that is at least 10-fold greater at the following receptors than at a GABA_{A} receptor: serotonin, dopamine, galanin, VR1, C5a, MCH, NPY, CRF, bradykinin and tackykinin. Assays to determine Kᵢ at other receptors may be performed using standard binding assay protocols, such as using a commercially available membrane receptor binding assay (e.g., the binding assays available from MDS PHARMA SERVICES, Toronto, Canada and CEREP, Redmond, Wash.).

A “CNS disorder” is a disease or condition of the central nervous system that is responsive to GABA_{A} receptor modulation in the patient. Such disorders include anxiety disorders (e.g., panic disorder, obsessive compulsive disorder, agoraphobia, social phobia, specific phobia, dysthymia, adjustment disorders, separation anxiety, cyclothymia and generalized anxiety disorder), stress disorders (e.g., post-traumatic stress disorder, anticipatory anxiety acute stress disorder and acute stress disorder), depressive disorders (e.g., depression, atypical depression, bipolar disorder and depressed phase of bipolar disorder), sleepwalking, sleep disorders (e.g., primary insomnia, circadian rhythm sleep disorder, dyssomnia NOS, parasomnias including nightmare disorder, sleep terror disorder, sleep disorders secondary to depression, anxiety and/or other mental disorders and substance-induced sleep disorder), cognitive disorders (e.g., cognition impairment, mild cognitive impairment (MCI), age-related cognitive decline (ARCD), schizophrenia, traumatic brain injury, Down's Syndrome, neurodegenerative diseases such as Alzheimer's disease and Parkinson's disease, and stroke), AIDS-associated dementia, dementia associated with depression, anxiety or psychosis, attention deficit disorders (e.g., attention deficit disorder and attention deficit and hyperactivity disorder), convulsive disorders (e.g., epilepsy), pain, benzodiazepine overdose and drug and alcohol addiction.

A “CNS agent” is any drug used to treat or prevent a CNS disorder or to induce or prolong sleep in a healthy patient. CNS agents include, for example: GABA_{A} receptor modulators, serotonin receptor (e.g., 5-HT_{1A}) agonists and antagonists and selective serotonin reuptake inhibitors (SSRIs); neurokinin receptor antagonists; corticotropin releasing factor receptor (CRF₁) antagonists; melatonin receptor agonists; nicotinic agonists; muscarinic agents; acetylcholinesterase inhibitors and dopamine receptor agonists.

A “therapeutically effective amount” (or dose) is an amount that, upon administration to a patient, results in a discernible patient benefit (e.g., diminution of one or more symptoms of a CNS disorder or inducing a desired effect on sleep). Such an amount or dose generally results in a concentration of compound in cerebrospinal fluid that is sufficient to inhibit the binding of GABA_{A} receptor ligand to GABA_{A} receptor in vitro, as determined using the assay described in Example 8. It will be apparent that the therapeutically effective amount for a compound will depend upon the indication for which the compound is administered, as well as any co-administration of other CNS agent(s). It will be apparent that the discernible patient benefit may be apparent after administration of a single dose, or may become apparent following repeated administration of the therapeutically effective dose according to a prescribed regimen, depending upon the indication for which the compound is administered.

A “patient” is any individual treated with a compound provided herein. Patients include humans, as well as other vertebrate animals such as companion animals and livestock. Patients may be afflicted with a CNS disorder, or may be free of such a condition (i.e., treatment may be prophylactic or soporific).

Compounds of Formula I

As noted above, the present invention provides compounds of Formula I, with the variables as described above, as well as pharmaceutically acceptable salts of such compounds.

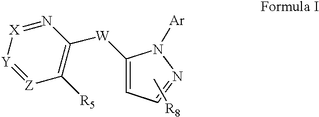

In certain compounds provided herein, R₈ represents 0 substituents or 1 substituent selected from halogen, C₁-C₂alkyl and C₁-C₂alkoxy.

Ar, within certain compounds of Formula I and other formulas provided herein, is substituted with 0, 1, 2 or 3 substituents independently selected from halogen, hydroxy, amino, cyano, aminocarbonyl, C₁-C₄alkyl, C₁-C₄alkoxy, mono- or di-(C₁-C₄alkyl)amino, C₂-C₄alkanoyl, (C₃-C₇cycloalkyl)C₀-C₂alkyl, C₁-C₄-aminoalkyl, C₁-C₄haloalkyl, C₁-C₄haloalkoxy and 5-membered heteroaryl. Certain Ar groups include phenyl, pyridyl, thiazolyl, thienyl, pyridazinyl and pyrimidinyl, each of which is substituted with from 0 to 3 substituents. Within certain embodiments, Ar represents phenyl, pyridyl, thiazolyl, thienyl or pyridazinyl, each of which is substituted with from 0 to 2 substituents (preferably 1 or 2 substituents) independently selected from halogen, hydroxy, cyano, amino, aminocarbonyl, C₁-C₄alkyl, C₁-C₄-aminoalkyl, C₁-C₄alkoxy, mono- or di-(C₁-C₂alkyl)amino, C₁-C₂haloalkyl, C₁-C₂haloalkoxy and 5-membered heteroaryl. Within further embodiments, Ar represents phenyl, pyridin-2-yl, 1,3-thiazol-2-yl, thien-2-yl or pyridazin-3-yl, each of which is substituted with from 0 to 3 substituents independently selected from fluoro, chloro, bromo, hydroxy, aminocarbonyl, thiazolyl, aminomethyl, methyl, ethyl, cyano, methoxy and ethoxy. Representative such Ar groups include, for example, 3-cyano-phenyl, pyridin-2-yl, 3-fluoro-pyridin-2-yl, 3-bromo-pyridin-2-yl, 3-chloro-pyridin-2-yl, 3-cyano-pyridin-2-yl, 3-aminomethyl-pyridin-2-yl, 3-aminocarbonyl-pyridin-2-yl, 3-thiazolyl-pyridin-2-yl, 6-fluoro-pyridin-2-yl and 6-cyano-pyridin-2-yl.

Within certain compounds of Formula I, and other formulas provided herein, R₁, R₂ and R₃ (if present) are independently selected from:

(a) hydrogen, halogen, nitro and cyano; and

(b) groups of the formula: 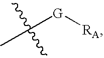
wherein:
(i) G is a single covalent bond, NH, N(R_{B}), O, C(═O)O or C(═O); and
(ii) R_{A} and R_{B} are independently selected from (1) hydrogen and (2) C₁-C₆alkyl, C₂-C₆alkenyl, (C₃-C₇cycloalkyl)C₀-C₄alkyl, (3- to 7-membered heterocycloalkyl)C₀-C₄alkyl, phenylC₀-C₄alkyl and (5- or 6-membered heteroaryl)C₀-C₄alkyl, each of which is substituted with from 0 to 4 substituents independently selected from hydroxy, halogen, cyano, amino, C₁-C₂alkyl and C₁-C₂alkoxy.

Within such compounds (and others in which L is absent), if G is a single covalent bond, then the group of the formula

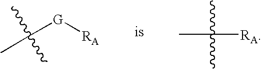

In certain such compounds, R₁, R₂ and R₃ are independently selected from hydrogen, hydroxy, halogen, cyano, amino, aminocarbonyl, nitro, C₁-C₆alkyl, C₂-C₆alkenyl, C₁-C₆alkoxy, C₂-C₆alkyl ether, C₃-C₇cycloalkylC₀-C₄alkyl, C₃-C₇cycloalkylC₁-C₄alkoxy, C₁-C₄hydroxyalkyl, C₁-C₆haloalkyl, C₁-C₆haloalkoxy, mono- or di-(C₁-C₆alkyl)amino C₁-C₆alkanoyl, C₁-C₆alkoxycarbonyl, mono- and di-(C₁-C₄alkyl)amino, phenylC₀-C₄alkyl, phenylC₁-C₄alkoxy, thienyl, pyridyl, pyrimidinyl, thiazolyl and pyrazinyl. Representative R₁ groups include hydrogen, methyl and ethyl.

Certain compounds of Formula I further satisfy one of the following formulas:

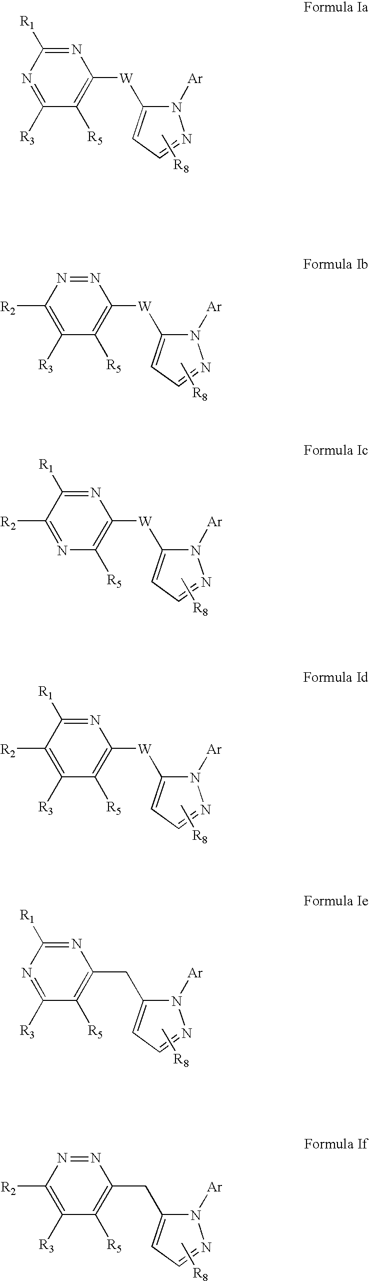

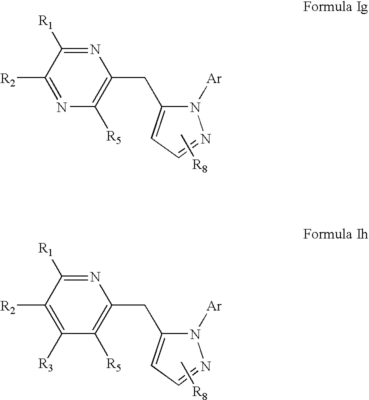

Certain compounds of Formula I further satisfy Formula II, in which Y is N, Z is CR₃, and R₃ is taken together with Y to form a fused 5-membered heteroaryl: 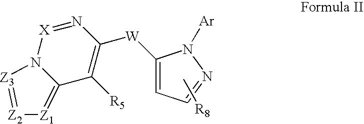
Within Formula II, Z₁, Z₂ and Z₃ are independently nitrogen or CR₄ such that exactly one or two of Z₁, Z₂ and Z₃ are nitrogen.

In certain such compounds, Z₁ and Z₃ are nitrogen and Z₂ is CR₄ (i.e., compounds of Formula IIa).

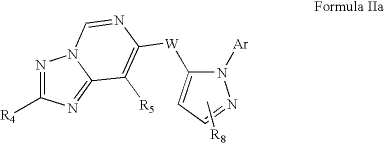

In other compounds of Formula II, Z₁ is nitrogen and Z₂ and Z₃ are independently chosen from CR₄ (i.e., compounds of Formula IIb).

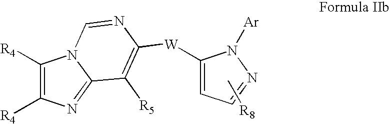

Within further compounds of Formula II, Z₁ is CR₄, Z₂ is nitrogen and Z₃ is CR₄ (i.e., compounds of Formula IIc).

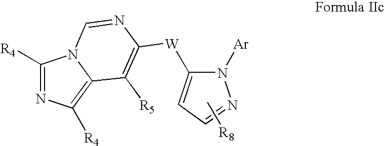

Within other compounds of Formula II, Z₁ and Z₂ are nitrogen and Z₃ is CR₄ (i.e., compounds of Formula IId).

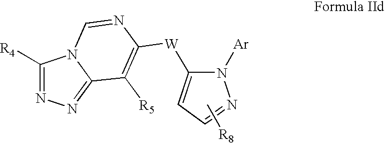

Within still further compounds of Formula II, Z₁ and Z₂ are CR₄, and Z₃ is nitrogen (i.e., compounds of Formula IIe).

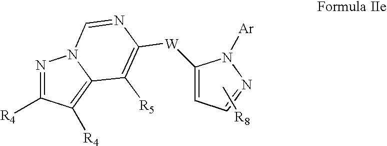

Within certain compounds of Formulas IIa-IIe, W is CH₂.

Certain compounds of Formula I further satisfy Formula III, in which Z is N, Y is CR₂, and R₂ is taken together with Z to form a fused 5-membered heteroaryl: 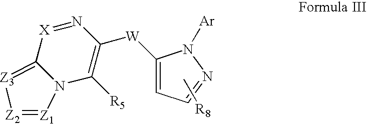
Within Formula III, Z₁, Z₂ and Z₃ are independently nitrogen or CR₄ such that exactly one or two of Z₁, Z₂ and Z₃ are nitrogen.

In certain such compounds (Formula IIIa), Z₁ is CR₄ and Z₂ and Z₃ are nitrogen.

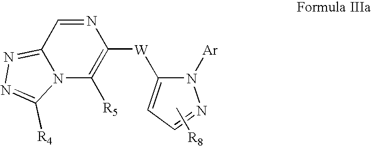

In other such compounds (Formula IIIb), Z₁ is nitrogen, Z₂ is CR₄ and Z₃ is nitrogen.

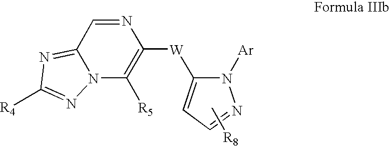

In further such compounds (Formula IIIc), Z₁ is nitrogen, and Z₂ and Z₃ are CR₄.

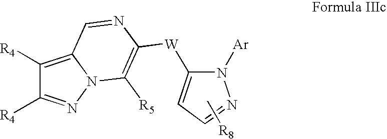

In other such compounds (Formula IIId), Z₁ is CR₄, Z₂ is nitrogen and Z₃ is CR₄.

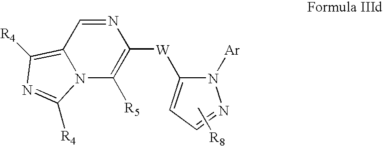

In further such compounds (Formula IIIe), Z₁ and Z₂ are CR₄, and Z₃ is nitrogen.

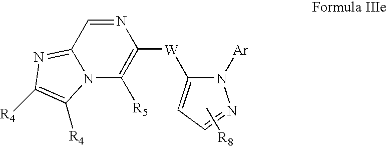

Within certain compounds of Formulas IIIa-IIIe, W is CH₂.

Within certain compounds of Formulas I, II, III and subformulas thereof, R₄ is: (a) hydrogen, halogen, cyano, amino or aminocarbonyl; or (b) C₁-C₄alkyl, C₁-C₄haloalkyl, C₁-C₄hydroxyalkyl, C₁-C₄alkoxy, C₁-C₄alkoxycarbonyl, C₂-C₄alkyl ether, (C₃-C₇cycloalkyl)C₀-C₂alkyl, mono- or di-(C₁-C₄alkyl)aminocarbonyl, (3- to 7-membered heterocycle)C₀-C₂alkyl or phenyl, each of which is substituted with from 0 to 2 substituents independently chosen from halogen, methyl and ethyl. Representative such R₄ groups include, for example, hydrogen, chloro, fluoro, cyano, amino, aminocarbonyl, methyl, ethyl, isopropyl, t-butyl, cyclopentylmethyl, methoxymethyl, ethoxymethyl, ethoxyethyl, hydroxymethyl, aminomethyl, methylaminocarbonyl, mono-, di- and tri-fluoromethyl, and (4- to 6-membered heterocycle)C₀-C₁alkyl (e.g., piperidinyl, morpholinyl, piperazinyl, morpholinylmethyl, piperidinylmethyl, piperazinylmethyl, pyrrolidinylmethyl, azetidinylmethyl or thiazolyl) that is optionally substituted with one or two substituents independently chosen from fluoro, chloro, methyl and ethyl.

W, within certain compounds of the above formulas, is CR₆R₇; preferably R₆ and R₇ are both hydrogen.

R₅, within certain compounds of the above formulas, is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy. Representative R₅ groups include ethyl, propyl, butyl, ethoxy and methoxymethyl.

Certain compounds of Formula I further satisfy Formula IV, in which X is N, Y is CR₂, wherein R₂ is taken together with X to form a fused 5-membered heteroaryl, and Z is CR₃: 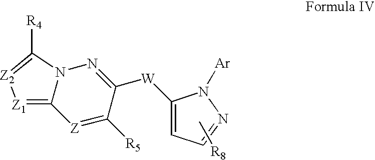
Within Formula IV, Z₁ and Z₂ are independently nitrogen or CR₄ such that exactly one or two of Z₁ and Z₂ are nitrogen.

Certain compounds of Formula IV further satisfy Formula IVa, in which Z₁ is N and Z₂ is CR₄.

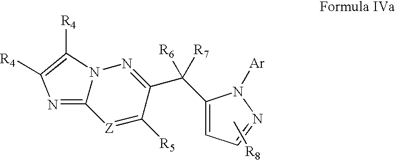

Certain compounds of Formula IV further satisfy Formula IVb, in which Z₁ is CR₄ and Z₂ is N.

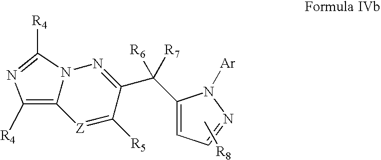

Other compounds of Formula IV further satisfy Formula IVc, in which Z₁ and Z₂ are nitrogen.

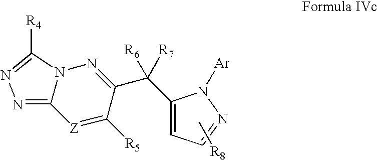

Within certain compounds of Formula IV, IVa, IVb or IVc, Z is CR₃; within further such compounds, R₆ and R₇ are both hydrogen.

Within certain compounds of Formulas I, II, III and IV and subformulas thereof:
R₁, if present, is hydrogen, methyl or ethyl;
R₅ is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy;
R₆ and R₇ are independently hydrogen, methyl, ethyl or halogen;
R₈ represents 0 or 1 substituent selected from halogen, C₁-C₂alkyl and C₁-C₂alkoxy; and/or
Ar represents phenyl, 2-pyridyl, 1,3-thiazol-2-yl, 2-thienyl or 3-pyridazinyl, each of which is substituted with from 0 to 2 substituents independently selected from fluoro, chloro, bromo, hydroxy, aminocarbonyl, thiazolyl, aminomethyl, methyl, ethyl, cyano, methoxy and ethoxy.

In certain aspects, compounds provided herein detectably alter (modulate) ligand binding to GABA_{A} receptor, as determined using a standard in vitro receptor binding assay. References herein to a “GABA_{A} receptor ligand binding assay” are intended to refer to the standard in vitro receptor binding assay provided in Example 8. Briefly, a competition assay may be performed in which a GABA_{A} receptor preparation is incubated with labeled (e.g., ³H) ligand, such as Flumazenil, and unlabeled test compound. Incubation with a compound that detectably modulates ligand binding to GABA_{A} receptor will result in a decrease or increase in the amount of label bound to the GABA_{A} receptor preparation, relative to the amount of label bound in the absence of the compound. Preferably, such a compound will exhibit a K; at GABA_{A} receptor of less than 1 micromolar, more preferably less than 500 nM, 100 nM, 20 nM or 10 nM. The GABA_{A} receptor used to determine in vitro binding may be obtained from a variety of sources, for example from preparations of rat cortex or from cells expressing cloned human GABA_{A} receptors.

In certain embodiments, preferred compounds provided herein have favorable pharmacological properties, including oral bioavailability (such that a sub-lethal or preferably a pharmaceutically acceptable oral dose, preferably less than 2 grams, more preferably less than or equal to one gram or 200 mg, can provide a detectable in vivo effect), low toxicity (a preferred compound is nontoxic when a therapeutically effective amount is administered to a subject), minimal side effects (a preferred compound produces side effects comparable to placebo when a therapeutically effective amount of the compound is administered to a subject), low serum protein binding, and a suitable in vitro and in vivo half-life (a preferred compound exhibits an in vivo half-life allowing for Q.I.D. dosing, preferably T.I.D. dosing, more preferably B.I.D. dosing and most preferably once-a-day dosing). Distribution in the body to sites of complement activity is also desirable (e.g., compounds used to treat CNS disorders will preferably penetrate the blood brain barrier, while low brain levels of compounds used to treat periphereal disorders are typically preferred).

Routine assays that are well known in the art may be used to assess these properties and identify superior compounds for a particular use. For example, assays used to predict bioavailability include transport across human intestinal cell monolayers, such as Caco-2 cell monolayers. Penetration of the blood brain barrier of a compound in humans may be predicted from the brain levels of the compound in laboratory animals given the compound (e.g., intravenously). Serum protein binding may be predicted from albumin binding assays, such as those described by Oravcová, et al. (1996) Journal of Chromatography B 677:1-27. Compound half-life is inversely proportional to the required frequency of dosage. In vitro half-lives of compounds may be predicted from assays of microsomal half-life as described by Kuhnz and Gieschen (1998) Drag Metabolism and Disposition 26:1120-27.

As noted above, preferred compounds provided herein are nontoxic. In general, the term “nontoxic” as used herein shall be understood in a relative sense and is intended to refer to any substance that has been approved by the United States Food and Drug Administration (“FDA”) for administration to mammals (preferably humans) or, in keeping with established criteria, is susceptible to approval by the FDA for administration to mammals (preferably humans). In addition, a highly preferred nontoxic compound generally satisfies one or more of the following criteria when administered at a minimum therapeutically effective amount or when contacted with cells at a concentration that is sufficient to inhibit the binding of GABA_{A} receptor ligand to GABA_{A} receptor in vitro: (1) does not substantially inhibit cellular ATP production; (2) does not significantly prolong heart QT intervals; (3) does not cause substantial liver enlargement or (4) does not cause substantial release of liver enzymes.

As used herein, a compound that does not substantially inhibit cellular ATP production is a compound that, when tested as described in Example 10, does not decrease cellular ATP levels by more than 50%. Preferably, cells treated as described in Example 10 exhibit ATP levels that are at least 80% of the ATP levels detected in untreated cells. Highly preferred compounds are those that do not substantially inhibit cellular ATP production when the concentration of compound is at least 10-fold, 100-fold or 1000-fold greater than the EC₅₀ or IC₅₀ for the compound.

A compound that does not significantly prolong heart QT intervals is a compound that does not result in a statistically significant prolongation of heart QT intervals (as determined by electrocardiography) in guinea pigs, minipigs or dogs upon administration of a dose that yields a serum concentration equal to the EC₅₀ or IC₅₀ for the compound. In certain preferred embodiments, a dose of 0.01, 0.05. 0.1, 0.5, 1, 5, 10, 40 or 50 mg/kg administered parenterally or orally does not result in a statistically significant prolongation of heart QT intervals. By “statistically significant” is meant results varying from control at the p<0.1 level or more preferably at the p<0.05 level of significance as measured using a standard parametric assay of statistical significance such as a student's T test.

A compound does not cause substantial liver enlargement if daily treatment of laboratory rodents (e.g., mice or rats) for 5-10 days with a dose that yields a serum concentration equal to the EC₅₀ or IC₅₀ for the compound results in an increase in liver to body weight ratio that is no more than 100% over matched controls. In more highly preferred embodiments, such doses do not cause liver enlargement of more than 75% or 50% over matched controls. If non-rodent mammals (e.g., dogs) are used, such doses should not result in an increase of liver to body weight ratio of more than 50%, preferably not more than 25%, and more preferably not more than 10% over matched untreated controls. Preferred doses within such assays include 0.01, 0.05. 0.1, 0.5, 1, 5, 10, 40 or 50 mg/kg administered parenterally or orally.

Similarly, a compound does not promote substantial release of liver enzymes if administration of a dose that yields a serum concentration equal to the EC₅₀ or IC₅₀ for the compound does not elevate serum levels of ALT, LDH or AST in laboratory rodents by more than 3-fold (preferably no more than 2-fold) over matched mock-treated controls. In more highly preferred embodiments, such doses do not elevate such serum levels by more than 75% or 50% over matched controls. Alternately, a compound does not promote substantial release of liver enzymes if, in an in vitro hepatocyte assay, concentrations (in culture media or other such solutions that are contacted and incubated with hepatocytes in vitro) concentrations that are equal to the EC₅₀ or IC₅₀ for the compound do not cause detectable release of any of such liver enzymes into culture medium above baseline levels seen in media from matched mock-treated control cells. In more highly preferred embodiments, there is no detectable release of any of such liver enzymes into culture medium above baseline levels when such compound concentrations are two-fold, five-fold, and preferably ten-fold the EC₅₀ or IC₅₀ for the compound.

In other embodiments, certain preferred compounds do not inhibit or induce microsomal cytochrome P450 enzyme activities, such as CYP1A2 activity, CYP2A6 activity, CYP2C9 activity, CYP2C19 activity, CYP2D6 activity, CYP2E1 activity or CYP3A4 activity at a concentration equal to the EC₅₀ or IC₅₀ for the compound.

Certain preferred compounds are not clastogenic or mutagenic (e.g., as determined using standard assays such as the Chinese hamster ovary cell vitro micronucleus assay, the mouse lymphoma assay, the human lymphocyte chromosomal aberration assay, the rodent bone marrow micronucleus assay, the Ames test or the like) at a concentration equal to the EC₅₀ or IC₅₀ for the compound. In other embodiments, certain preferred compounds do not induce sister chromatid exchange (e.g., in Chinese hamster ovary cells) at such concentrations.

For detection purposes, as discussed in more detail below, compounds provided herein may be isotopically-labeled or radiolabeled. Such compounds are identical to those described above, but for the fact that one or more atoms are replaced by an atom having an atomic mass or mass number different from the atomic mass or mass number usually found in nature. Examples of isotopes that can be incorporated into compounds provided herein include isotopes of hydrogen, carbon, nitrogen, oxygen, phosphorous, fluorine and chlorine, such as ²H, ³H, ¹¹C, ¹³C, ¹⁴C, ¹⁵N, ¹⁸O, ¹⁷O, ³¹P, ³²P, ³⁵S, ¹⁸F and ³⁶Cl. In addition, substitution with heavy isotopes such as deuterium (i.e., ²H) can afford certain therapeutic advantages resulting from greater metabolic stability, such as increased in vivo half-life or reduced dosage requirements and, hence, may be preferred in some circumstances.

As noted above, different stereoisomeric forms, such as racemates and optically active forms, are encompassed by the present invention. In certain embodiments, it may be desirable to obtain single enantiomers (i.e., optically active forms). Standard methods for preparing single enantiomers include asymmetric synthesis and resolution of the racemates. Resolution of the racemates can be accomplished by conventional methods such as crystallization in the presence of a resolving agent, or chromatography using, for example, a chiral HPLC column.

Pharmaceutical Compositions

The present invention also provides pharmaceutical compositions comprising at least one compound or salt of Formula I, together with at least one physiologically acceptable carrier or excipient. Such pharmaceutical compositions may be used to treat patients in which GABA_{A} receptor modulation is desirable (e.g., patients undergoing painful procedures who would benefit from the induction of amnesia, or those suffering from CNS disorders such as anxiety, depression, sleepwalking, sleep disorders or cognitive impairment). Pharmaceutical compositions may comprise, for example, water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. Preferred pharmaceutical compositions are formulated for oral delivery to humans or other animals (e.g., companion animals such as dogs or cats). If desired, other active ingredients may also be included, such as additional CNS-active agents.

Pharmaceutical compositions may be formulated for any appropriate manner of administration, including, for example, inhalation (e.g., nasal or oral), topical, oral, nasal, rectal or parenteral administration. The term parenteral as used herein includes subcutaneous, intradermal, intravascular (e.g., intravenous), intramuscular, spinal, intracranial, intrathecal and intraperitoneal injection, as well as any similar injection or infusion technique. In certain embodiments, compositions in a form suitable for oral use are preferred. Such forms include, for example, tablets, troches, lozenges, aqueous or oily suspensions, dispersible powders or granules, emulsion, hard or soft capsules, or syrups or elixirs. Within yet other embodiments, compositions of the present invention may be formulated as a lyophilizate.

Compositions intended for oral use may further comprise one or more components such as sweetening agents, flavoring agents, coloring agents and preserving agents in order to provide appealing and palatable preparations. Tablets contain the active ingredient in admixture with physiologically acceptable excipients that are suitable for the manufacture of tablets. Such excipients include, for example, inert diluents to increase the bulk weight of the material to be tableted (e.g., calcium carbonate, sodium carbonate, lactose, calcium phosphate or sodium phosphate), granulating and disintegrating agents that modify the disintegration rate in the environment of use (e.g., corn starch, starch derivatives, alginic acid and salts of carboxymethylcellulose), binding agents that impart cohesive qualities to the powdered material(s) (e.g., starch, gelatin, acacia and sugars such as sucrose, glucose, dextrose and lactose) and lubricating agents (e.g., magnesium stearate, calcium stearate, stearic acid or talc). Tablets may be formed using standard techniques, including dry granulation, direct compression and wet granulation. The tablets may be uncoated or they may be coated by known techniques.

Formulations for oral use may also be presented as hard gelatin capsules wherein the active ingredient is mixed with an inert solid diluent (e.g., calcium carbonate, calcium phosphate or kaolin), or as soft gelatin capsules wherein the active ingredient is mixed with water or an oil medium (e.g., peanut oil, liquid paraffin or olive oil).

Aqueous suspensions comprise the active materials in admixture with one or more suitable excipients, such as suspending agents (e.g., sodium carboxymethylcellulose, methylcellulose, hydroxypropylmethylcellulose, sodium alginate, polyvinylpyrrolidone, gum tragacanth and gum acacia); and dispersing or wetting agents (e.g., naturally-occurring phosphatides such as lecithin, condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides such as polyethylene sorbitan monooleate). Aqueous suspensions may also contain one or more preservatives, such as ethyl, or n-propyl p-hydroxybenzoate, one or more coloring agents, one or more flavoring agents and/or one or more sweetening agents, such as sucrose or saccharin.

Oily suspensions may be formulated by suspending the active ingredients in a vegetable oil (e.g., arachis oil, olive oil, sesame oil or coconut oil) or in a mineral oil such as liquid paraffin. The oily suspensions may contain a thickening agent such as beeswax, hard paraffin or cetyl alcohol. Sweetening agents and/or flavoring agents may be added to provide palatable oral preparations. Such suspensions may be preserved by the addition of an anti-oxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified above. Additional excipients, such as sweetening, flavoring and coloring agents, may also be present.

Pharmaceutical compositions may also be in the form of oil-in-water emulsions. The oily phase may be a vegetable oil (e.g., olive oil or arachis oil) or a mineral oil (e.g., liquid paraffin) or a mixture thereof. Suitable emulsifying agents include naturally-occurring gums (e.g., gum acacia or gum tragacanth), naturally-occurring phosphatides (e.g., soy bean, lecithin, and esters or partial esters derived from fatty acids and hexitol), anhydrides (e.g., sorbitan monoleate) and condensation products of partial esters derived from fatty acids and hexitol with ethylene oxide (e.g., polyoxyethylene sorbitan monoleate). An emulsion may also comprise one or more sweetening and/or flavoring agents.

Syrups and elixirs may be formulated with sweetening agents, such as glycerol, propylene glycol, sorbitol or sucrose. Such formulations may also comprise one or more demulcents, preservatives, flavoring agents and/or coloring agents.

A pharmaceutical composition may be prepared as a sterile injectible aqueous or oleaginous suspension. The compound(s) provided herein, depending on the vehicle and concentration used, can either be suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid find use in the preparation of injectible compositions, and adjuvants such as local anesthetics, preservatives and/or buffering agents can be dissolved in the vehicle.

Pharmaceutical compositions may also be prepared in the form of suppositories (e.g., for rectal administration). Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

Pharmaceutical compositions may be formulated as controlled release formulations (i.e., a formulation such as a capsule, tablet or coated tablet that slows and/or delays release of active ingredient(s) following administration), which may be administered by, for example, oral, rectal or subcutaneous implantation, or by implantation at a target site. In general, a controlled release formulation comprises a matrix and/or coating that delays disintegration and absorption in the gastrointestinal tract (or implantation site) and thereby provides a delayed action or a sustained action over a longer period. One type of controlled-release formulation is a sustained-release formulation, in which at least one active ingredient is continuously released over a period of time at a constant rate. Preferably, the therapeutic agent is released at such a rate that blood (e.g., plasma) concentrations are maintained within the therapeutic range, but below toxic levels, over a period of time that is at least 4 hours, preferably at least 8 hours, and more preferably at least 12 hours.

Controlled release may be achieved by combining the active ingredient(s) with a matrix material that itself alters release rate and/or through the use of a controlled-release coating. The release rate can be varied using methods well known in the art, including (a) varying the thickness or composition of coating, (b) altering the amount or manner of addition of plasticizer in a coating, (c) including additional ingredients, such as release-modifying agents, (d) altering the composition, particle size or particle shape of the matrix, and (e) providing one or more passageways through the coating. The amount of modulator contained within a sustained release formulation depends upon, for example, the method of administration (e.g., the site of implantation), the rate and expected duration of release and the nature of the condition to be treated or prevented.

The matrix material, which itself may or may not serve a controlled-release function, is generally any material that supports the active ingredient(s). For example, a time delay material such as glyceryl monosterate or glyceryl distearate may be employed. Active ingredient(s) may be combined with matrix material prior to formation of the dosage form (e.g., a tablet). Alternatively, or in addition, active ingredient(s) may be coated on the surface of a particle, granule, sphere, microsphere, bead or pellet that comprises the matrix material. Such coating may be achieved by conventional means, such as by dissolving the active ingredient(s) in water or other suitable solvent and spraying. Optionally, additional ingredients are added prior to coating (e.g., to assist binding of the active ingredient(s) to the matrix material or to color the solution). The matrix may then be coated with a barrier agent prior to application of controlled-release coating. Multiple coated matrix units may, if desired, be encapsulated to generate the final dosage form.

In certain embodiments, a controlled release is achieved through the use of a controlled release coating (i.e., a coating that permits release of active ingredient(s) at a controlled rate in aqueous medium). The controlled release coating should be a strong, continuous film that is smooth, capable of supporting pigments and other additives, non-toxic, inert and tack-free. Coatings that regulate release of the modulator include pH-independent coatings, pH-dependent coatings (which may be used to release modulator in the stomach) and enteric coatings (which allow the formulation to pass intact through the stomach and into the small intestine, where the coating dissolves and the contents are absorbed by the body). It will be apparent that multiple coatings may be employed (e.g., to allow release of a portion of the dose in the stomach and a portion further along the gastrointestinal tract). For example, a portion of active ingredient(s) may be coated over an enteric coating, and thereby released in the stomach, while the remainder of active ingredient(s) in the matrix core is protected by the enteric coating and released further down the GI tract. pH dependent coatings include, for example, shellac, cellulose acetate phthalate, polyvinyl acetate phthalate, hydroxypropylmethylcellulose phthalate, methacrylic acid ester copolymers and zein.

In certain embodiments, the coating is a hydrophobic material, preferably used in an amount effective to slow the hydration of the gelling agent following administration. Suitable hydrophobic materials include alkyl celluloses (e.g., ethylcellulose or carboxymethylcellulose), cellulose ethers, cellulose esters, acrylic polymers (e.g., poly(acrylic acid), poly(methacrylic acid), acrylic acid and methacrylic acid copolymers, methyl methacrylate copolymers, ethoxy ethyl methacrylates, cyanoethyl methacrylate, methacrylic acid alkamide copolymer, poly(methyl methacrylate), polyacrylamide, ammonio methacrylate copolymers, aminoalkyl methacrylate copolymer, poly(methacrylic acid anhydride) and glycidyl methacrylate copolymers) and mixtures of the foregoing. Representative aqueous dispersions of ethylcellulose include, for example, AQUACOAT® (FMC Corp., Philadelphia, Pa.) and SURELEASE® (Colorcon, Inc., West Point, Pa.), both of which can be applied to the substrate according to the manufacturer's instructions. Representative acrylic polymers include, for example, the various EUDRAGIT® (Rohm America, Piscataway, N.J.) polymers, which may be used singly or in combination depending on the desired release profile, according to the manufacturer's instructions.

The physical properties of coatings that comprise an aqueous dispersion of a hydrophobic material may be improved by the addition or one or more plasticizers. Suitable plasticizers for alkyl celluloses include, for example, dibutyl sebacate, diethyl phthalate, triethyl citrate, tributyl citrate and triacetin. Suitable plasticizers for acrylic polymers include, for example, citric acid esters such as triethyl citrate and tributyl citrate, diputyl phthalate, polyethylene glycols, propylene glycol, diethyl phthalate, castor oil and triacetin.

Controlled-release coatings are generally applied using conventional techniques, such as by spraying in the form of an aqueous dispersion. If desired, the coating may comprise pores or channels or to facilitate release of active ingredient. Pores and channels may be generated by well known methods, including the addition of organic or inorganic material that is dissolved, extracted or leached from the coating in the environment of use. Certain such pore-forming materials include hydrophilic polymers, such as hydroxyalkylcelluloses (e.g., hydroxypropylmethylcellulose), cellulose ethers, synthetic water-soluble polymers (e.g., polyvinylpyrrolidone, cross-linked polyvinylpyrrolidone and polyethylene oxide), water-soluble polydextrose, saccharides and polysaccharides and alkali metal salts. Alternatively, or in addition, a controlled release coating may include one or more orifices, which may be formed my methods such as those described in U.S. Pat. Nos. 3,845,770; 4,034,758; 4,077,407; 4,088,864; 4,783,337 and 5,071,607. Controlled-release may also be achieved through the use of transdermal patches, using conventional technology (see, e.g., U.S. Pat. No. 4,668,232).

Further examples of controlled release formulations, and components thereof, may be found, for example, in U.S. Pat. Nos. 5,524,060; 4,572,833; 4,587,117; 4,606,909; 4,610,870; 4,684,516; 4,777,049; 4,994,276; 4,996,058; 5,128,143; 5,202,128; 5,376,384; 5,384,133; 5,445,829; 5,510,119; 5,618,560; 5,643,604; 5,891,474; 5,958,456; 6,039,980; 6,143,353; 6,126,969; 6,156,342; 6,197,347; 6,387,394; 6,399,096; 6,437,000; 6,447,796; 6,475,493; 6,491,950; 6,524,615; 6,838,094; 6,905,709; 6,923,984; 6,923,988; and 6,911,217; each of which is hereby incorporated by reference for its teaching of the preparation of controlled release dosage forms.

In addition to or together with the above modes of administration, a compound provided herein may be conveniently added to food or drinking water (e.g., for administration to non-human animals including companion animals (such as dogs and cats) and livestock). Animal feed and drinking water compositions may be formulated so that the animal takes in an appropriate quantity of the composition along with its diet. It may also be convenient to present the composition as a premix for addition to feed or drinking water.

Compounds provided herein are generally present within a pharmaceutical composition in a therapeutically effective amount, as described above. Compositions providing dosage levels ranging from about 0.1 mg to about 140 mg per kilogram of body weight per day are preferred (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. It will be understood, however, that the optimal dose for any particular patient will depend upon a variety of factors, including the activity of the specific compound employed; the age, body weight, general health, sex and diet of the patient; the time and route of administration; the rate of excretion; any simultaneous treatment, such as a drug combination; and the type and severity of the particular disease undergoing treatment. Optimal dosages may be established using routine testing and procedures that are well known in the art.

Pharmaceutical compositions may be packaged for treating a CNS disorder such as anxiety, depression, sleepwalking, a sleep disorder, attention deficit disorder or a cognitive disorder such as short-term memory loss or Alzheimer's dementia. Packaged pharmaceutical preparations include a container holding a therapeutically effective amount of at least one compound as described herein and instructions (e.g., labeling) indicating that the contained composition is to be used for treating the CNS disorder.

Methods of Use

Within certain aspects, the present invention provides methods for inhibiting the development of a CNS disorder. In other words, therapeutic methods provided herein may be used to treat an existing disorder, or may be used to prevent, decrease the severity of, or delay the onset of such a disorder in a patient who is free of detectable CNS disorder. CNS disorders are discussed in more detail below, and may be diagnosed and monitored using criteria that have been established in the art. Alternatively, or in addition, compounds provided herein may be administered to a patient to improve short-term memory or induce sleep in a healthy patient. Patients include humans, domesticated companion animals (pets, such as dogs) and livestock animals, with dosages and treatment regimes as described above.

Frequency of dosage may vary, depending on the compound used and the particular disease to be treated or prevented. In general, for treatment of most disorders, a dosage regimen of 4 times daily or less is preferred. For soporific treatment, a single dose that rapidly reaches a concentration in cerebrospinal fluid that is sufficient to inhibit the binding of GABA_{A} receptor ligand to GABA_{A} receptor in vitro is desirable. Patients may generally be monitored for therapeutic effectiveness using assays suitable for the condition being treated or prevented, which will be familiar to those of ordinary skill in the art.

Within preferred embodiments, compounds provided herein are used to treat patients with an existing CNS disorder. In general, such patients are treated with a therapeutically effective amount of a compound of Formula I (or a pharmaceutically acceptable salt thereof); preferably the amount is sufficient to alter one or more symptoms of a CNS disorder. Compounds that act as agonists at α₂β₃γ₂ and α₃β₃γ₂ receptor subtypes are particularly useful in treating anxiety disorders such as panic disorder, obsessive compulsive disorder and generalized anxiety disorder; stress disorders including post-traumatic stress and acute stress disorders. Compounds that act as agonists at α₂β₃γ₂ and α₃β₃γ₂ receptor subtypes are also useful in treating depressive or bipolar disorders, schizophrenia and sleep disorders, and may be used in the treatment of age-related cognitive decline and Alzheimer's disease. Compounds that act as inverse agonists at the α₅β₃γ₂ receptor subtype or α₁β₂γ₂ and α₅β₃γ₂ receptor subtypes are particularly useful in treating cognitive disorders including those resulting from Down's Syndrome, neurodegenerative diseases such as Alzheimer's disease, Parkinson's disease and stroke related dementia. Compounds that act as inverse agonists at the α₅β_{3 g2} receptor subtype are particularly useful in treating cognitive disorders through the enhancement of memory, particularly short-term memory, in memory-impaired patients; while those that act as agonists at the α₅β_{3 g2} receptor subtype are particularly useful for the induction of amnesia. Compounds that act as agonists at the α₁β₂γ₂ receptor subtype are useful in treating sleep disorders and convulsive disorders such as epilepsy. Compounds that act as antagonists at the benzodiazepine site are useful in reversing the effect of benzodiazepine overdose and in treating drug and alcohol addiction.

CNS disorders that can be treated using compounds and compositions provided herein include:
Depression, e.g., major depression, dysthymic disorder, atypical depression, bipolar disorder and depressed phase of bipolar disorder.
Anxiety, e.g., general anxiety disorder (GAD), agoraphobia, panic disorder +/−agoraphobia, social phobia, specific phobia, post traumatic stress disorder, obsessive compulsive disorder (OCD), dysthymia, adjustment disorders with disturbance of mood and anxiety, separation anxiety disorder, anticipatory anxiety acute stress disorder, adjustment disorders and cyclothymia.
Sleepwalking and Sleep disorders, e.g., primary insomnia, circadian rhythm sleep disorder, dyssomnia NOS, parasomnias, including nightmare disorder, sleep terror disorder, sleep disorders secondary to depression and/or anxiety or other mental disorders, and substance induced sleep disorder. Representative treatable symptoms of sleep disorders include, for example, difficulty falling asleep, excessive waking during the night, waking too early and waking feeling unrefreshed.
Neurodegenerative Disorders, Cognition Impairment, and Acute Brain Injuries, e.g., Alzheimer's disease, Huntington's disease, Parkinson's disease, Amyotrophic Lateral Sclerosis (ALS), reflex sympathetic dystrophy (RSD), spastic paralysis, hypokinesia, mild cognitive impairment (MCI), age-related cognitive decline (ARCD), stroke (e.g., acute thromboembolic stroke, focal ischemia, global ischemia, transient cerebral ischemic attacks and other cerebrovascular problems accompanied by cerebral ischemia), traumatic brain injury, spinal cord trauma, asphyxia, injury from general anoxia, hypoxia, hypoglycemia or hypotension, AIDS associated dementia, and dementia associated with depression, anxiety and psychosis (including schizophrenia and hallucinatory disorders).
Attention Deficit Disorders, e.g., attention deficit disorder (ADD) and attention deficit and hyperactivity disorder (ADHD).
Acute or Chronic Pain, e.g., postoperative pain, hyperalgesia (e.g., thermal or mechanical), allodynia (e.g., mechanical or cold-induced allodynia), somatogenic pains, psychogenic pains (e.g., low back pain, atypical facial pain, and chronic headache), nociceptive pain, pain caused by injury or inflammation of peripheral sensory nerves (e.g., pain from peripheral nerve trauma, herpes virus infection, diabetes mellitus, causalgia, plexus avulsion, neuroma, limb amputation, and vasculitis), neuropathic pain (e.g., caused by nerve damage from chronic alcoholism, human immunodeficiency virus infection, hypothyroidism, uremia, diabetes or vitamin deficiencies), inflammatory pain, osteoarthritic pain, erythromelalgic pain, post-poliomyelitic pain, trigeminal neuralgia, post-herpetic neuralgia, cancer pain, diabetic neuropathy, acute herpetic and postherpetic neuralgia, causalgia, brachial plexus avulsion, occipital neuralgia, gout, phantom limb, bum, and other forms of neuralgia, neuropathic, idiopathic pain syndrome, migraine, and pain caused by multiple sclerosis, acquired nystagmus or painful diabetic neuropathy.
Speech and Movement Disorders, e.g., motor tic, clonic stuttering, dysfluency, speech blockage, dysarthria, Tourette's Syndrome and logospasm, restless leg syndrome, periodic limb movements in sleep (PLMS), periodic limb movement disorder (PLMD), muscle spasm, essential tremor, acquired nystagmus, post-anoxic myoclonus, spinal myoclonus, spasticity, chorea and dystonia.
Convulsive disorders e.g., epilepsy.

Compounds and compositions provided herein can also be used to improve short-term memory (working memory) in a patient. A preferred therapeutically effective amount of a compound for improving short-term memory loss is an amount sufficient to result in a statistically significant improvement in any standard test of short-term memory function, including forward digit span and serial rote learning. For example, such a test may be designed to evaluate the ability of a patient to recall words or letters. Alternatively, a more complete neurophysical evaluation may be used to assess short-term memory function. Patients treated in order to improve short-term memory may, but need not, have been diagnosed with memory impairment or be considered predisposed to development of such impairment.

In a separate aspect, the present invention provides methods for potentiating the action (or therapeutic effect) of other CNS agent(s). Such methods comprise administering a therapeutically effective amount of a compound provided herein in combination with a therapeutically effective amount of another CNS agent. Such other CNS agents include, but are not limited to the following: for anxiety, serotonin receptor (e.g., 5-HT_{1A}) agonists and antagonists; for anxiety and depression, neurokinin receptor antagonists or corticotropin releasing factor receptor (CRF₁) antagonists; for sleep disorders, melatonin receptor agonists; and for neurodegenerative disorders, such as Alzheimer's dementia, nicotinic agonists, muscarinic agents, acetylcholinesterase inhibitors and dopamine receptor agonists. Within certain embodiments, the present invention provides a method of potentiating the antidepressant activity of selective serotonin reuptake inhibitors (SSRIs) by co-administering a therapeutically effective amount of a GABA_{A} agonist compound provided herein in combination with an SSRI. A therapeutically effective amount of compound, when co-administered with another CNS agent, is an amount sufficient to result in a detectable change in patient symptoms, when compared to a patient treated with the other CNS agent alone.

The present invention also pertains to methods of inhibiting the binding of benzodiazepine compounds (i.e., compounds that comprise the benzodiazepine ring structure), such as RO15-1788 or GABA, to GABA_{A} receptor. Such methods involve contacting cells expressing GABA_{A} receptor with a concentration of compound provided herein that is sufficient to inhibit the binding of GABA_{A} receptor ligand to GABA_{A} receptor in vitro, as determined using the assay described in Example 8. This method includes, but is not limited to, inhibiting the binding of benzodiazepine compounds to GABA_{A} receptors in vivo (e.g., in a patient given an amount of a GABA_{A} receptor modulator provided herein that results in a concentration of compound in cerebrospinal fluid that is sufficient to inhibit the binding of benzodiazepine compounds or GABA to GABA_{A} receptor in vitro). In one embodiment, such methods are useful in treating benzodiazepine drug overdose. The amount of GABA_{A} receptor modulator that is sufficient to inhibit the binding of a benzodiazepine compound to GABA_{A} receptor may be readily determined via a GABA_{A} receptor binding assay as described in Example 8.

Within separate aspects, the present invention provides a variety of in vitro uses for the GABA_{A} receptor modulators provided herein. For example, such compounds may be used as probes for the detection and localization of GABA_{A} receptors, in samples such as tissue sections, as positive controls in assays for receptor activity, as standards and reagents for determining the ability of a candidate agent to bind to GABA_{A} receptor, or as radiotracers for positron emission tomography (PET) imaging or for single photon emission computerized tomography (SPECT). Such assays can be used to characterize GABA_{A} receptors in living subjects. Such compounds are also useful as standards and reagents in determining the ability of a potential pharmaceutical to bind to GABA_{A} receptor.

Within methods for determining the presence or absence of GABA_{A} receptor in a sample, a sample may be incubated with a compound as provided herein under conditions that permit binding of the compound to GABA_{A} receptor. The amount of compound bound to GABA_{A} receptor in the sample is then detected. For example, the compound may be labeled using any of a variety of well known techniques (e.g., radiolabeled with a radionuclide such as tritium, as described herein), and incubated with the sample (which may be, for example, a preparation of cultured cells, a tissue preparation or a fraction thereof). A suitable incubation time may generally be determined by assaying the level of binding that occurs over a period of time. Following incubation, unbound compound is removed, and bound compound detected using any method suitable for the label employed (e.g., autoradiography or scintillation counting for radiolabeled compounds; spectroscopic methods may be used to detect luminescent groups and fluorescent groups). As a control, a matched sample may be simultaneously contacted with radiolabeled compound and a greater amount of unlabeled compound. Unbound labeled and unlabeled compound is then removed in the same fashion, and bound label is detected. A greater amount of detectable label in the test sample than in the control indicates the presence of GABA_{A} receptor in the sample. Detection assays, including receptor autoradiography (receptor mapping) of GABA_{A} receptors in cultured cells or tissue samples may be performed as described by Kuhar in sections 8.1.1 to 8.1.9 of Current Protocols in Pharmacology (1998) John Wiley & Sons, New York.

For example, compounds provided herein may be used for detecting GABA_{A} receptors in cell or tissue samples. This may be done using matched cell or tissue samples that have not previously been contacted with a GABA_{A} receptor modulator, at least one of which is prepared as an experimental sample and at least one of which is prepared as a control sample. An experimental sample is prepared by contacting (under conditions that permit binding of RO15-1788 to GABA_{A} receptors within cell and tissue samples) a sample with a detectably-labeled compound of Formula I. A control sample is prepared in the same manner as the experimental sample, except that it is also is contacted with unlabelled compound at a molar concentration that is greater than the concentration of labeled modulator.

The experimental and control samples are then washed to remove unbound detectably-labeled compound. The amount of remaining bound detectably-labeled compound is then measured and the amount of detectably-labeled compound in the experimental and control samples is compared. The detection of a greater amount of detectable label in the washed experimental sample(s) than in the washed control sample(s) demonstrates the presence of GABA_{A} receptor in the experimental sample.

The detectably-labeled GABA_{A} receptor modulator used in this procedure may be labeled with a radioactive label or a directly or indirectly luminescent label. When tissue sections are used in this procedure and the label is a radiolabel, the bound, labeled compound may be detected autoradiographically.

Compounds provided herein may also be used within a variety of well known cell culture and cell separation methods. For example, compounds may be linked to the interior surface of a tissue culture plate or other cell culture support, for use in immobilizing GABA_{A} receptor-expressing cells for screens, assays and growth in culture. Such linkage may be performed by any suitable technique, such as the methods described above, as well as other standard techniques. Compounds may also be used to facilitate cell identification and sorting in vitro, permitting the selection of cells expressing a GABA_{A} receptor. Preferably, the compound(s) for use in such methods are labeled as described herein. Within one preferred embodiment, a compound linked to a fluorescent marker, such as fluorescein, is contacted with the cells, which are then analyzed by fluorescence activated cell sorting (FACS).

Within other aspects, methods are provided for modulating binding of ligand to a GABA_{A} receptor in vitro or in vivo, comprising contacting a GABA_{A} receptor with a sufficient amount of a GABA_{A} receptor modulator provided herein, under conditions suitable for binding of ligand to the receptor. The GABA_{A} receptor may be present in solution, in a cultured or isolated cell preparation or within a patient. Preferably, the GABA_{A} receptor is a present in the brain of a mammal. In general, the amount of compound contacted with the receptor should be sufficient to modulate ligand binding to GABA_{A} receptor in vitro within, for example, a binding assay as described in Example 8.

Also provided herein are methods for altering the signal-transducing activity of cellular GABA_{A} receptor (particularly the chloride ion conductance), by contacting GABA_{A} receptor, either in vitro or in vivo, with a sufficient amount of a compound as described above, under conditions suitable for binding of Flumazenil to the receptor. The GABA_{A} receptor may be present in solution, in a cultured or isolated cell or cell membrane preparation or within a patient, and the amount of compound may be an amount that would be sufficient to alter the signal-transducing activity of GABA_{A} receptor in vitro. In certain embodiments, the amount or concentration of compound contacted with the receptor should be sufficient to modulate Flumazenil binding to GABA_{A} receptor in vitro within, for example, a binding assay as described in Example 8. An effect on signal-transducing activity may be detected as an alteration in the electrophysiology of the cells, using standard techniques. The amount or concentration of a compound that is sufficient to alter the signal-transducing activity of GABA_{A} receptors may be determined via a GABA_{A} receptor signal transduction assay, such as the assay described in Example 9. The cells expressing the GABA receptors in vivo may be, but are not limited to, neuronal cells or brain cells. Such cells may be contacted with one or more compounds provided herein through contact with a body fluid containing the compound, for example through contact with cerebrospinal fluid. Alteration of the signal-transducing activity of GABA_{A} receptors in cells in vitro may be determined from a detectable change in the electrophysiology of cells expressing GABA_{A} receptors, when such cells are contacted with a compound as described herein in the presence of GABA.

Intracellular recording or patch-clamp recording may be used to quantitate changes in electrophysiology of cells. A reproducible change in behavior of an animal given a compound as described herein may also be taken to indicate that a change in the electrophysiology of the animal's cells expressing GABA_{A} receptors has occurred.

Preparation of Compounds

Compounds provided herein may generally be prepared using standard synthetic methods. Representative procedures suitable for the preparation of compounds provided herein are outlined in the following Schemes, which are not to be construed as limiting the invention in scope or spirit to the specific reagents and conditions shown in them. Those having skill in the art will recognize that the reagents and conditions may be varied and additional steps employed to produce compounds encompassed by the present invention. In some cases, protection of reactive functionalities may be necessary to achieve the desired transformations. In general, such need for protecting groups, as well as the conditions necessary to attach and remove such groups, will be apparent to those skilled in the art of organic synthesis. Each variable in the following schemes refers to any group consistent with the description of the compounds provided herein.

Unless otherwise indicated, starting materials are generally readily available from commercial sources, such as Sigma-Aldrich Corp. (St. Louis, Mo.), and various intermediates may be obtained from commercial sources, prepared from commercially available organic compounds, or prepared using known synthetic methods. Representative examples of methods suitable for preparing intermediates are set forth below in Examples 1-7.

Abbreviations Used

| | |
|---|---|
| Ac | acetyl or acetate |
| BINAP | (rac)-2,2′-Bis(diphenylphosphino)-1,1′-binaphthyl |
| Bu | butyl |
| BuLi | n-butyl lithium |
| Bu₃Sn | tributyl tin |
| CDCl₃ | deuterated chloroform |
| δ | chemical shift |
| DCM | dichloromethane |
| DMF | N,N-dimethylformamide |
| DMSO | dimethylsulfoxide |
| DPPF | 1,1′-bis(diphenylphosphino)ferrocene |
| EtOAc | ethyl acetate |
| EtOH | ethanol |
| eq. | equivalent(s) |
| HOAc | acetic acid |
| HMPA | hexamethylphosphoramide |
| HPLC | high pressure liquid chromatography |
| ¹H NMR | proton nuclear magnetic resonance |
| Hz | hertz |
| LC-MS | liquid chromatography/mass spectrometry |
| LDA | lithium diisopropylamide |
| mCPBA | m-chloroperoxybenzoic acid |
| MeOH | methanol |
| MS | mass spectrometry |
| M + 1 | mass + 1 |
| NaOMe | sodium methoxide |
| Nu | nucleophile |
| OEt | ethoxy |
| Pd/C | palladium carbon catalyst |
| Pd(PPh₃)₄ | tetrakis(triphenylphosphine) palladium (0) |
| Pd(Ph₃P)₂Cl₂ | dichlorobis(triphenylphosphine) palladium (II) |
| Pd₂(dba)₃ | tris(dibenzylidineacetone) dipalladium (0) |
| Ph₃P (or PPh₃) | triphenylphosphine |
| PTLC | preparative thin layer chromatography |
| THF | tetrahydrofuran |
| TLC | thin layer chromatography |

Reaction Schemes

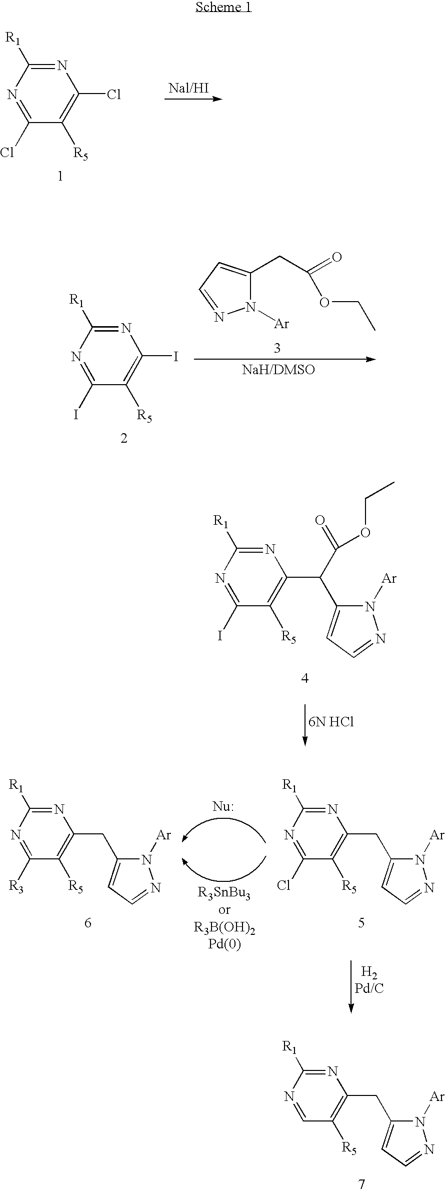

Scheme 1 illustrates the synthesis of compounds of formulas 6 and 7. Compound 1 is prepared essentially as described in J. Heterocycl. Chem. (1974) 11:295-297. Compound 3 is prepared essentially as described in Chem. Ber. (1985) 118:741-752 or Farmaco (1990) 45:167-186. Iodination of 1 with NaI/HI affords 2. Treatment of 3 with NaH in DMSO, followed by reaction with 2, provides 4. Hydrolysis and decarboxylation of 4 with 6 N HCl gives 5. Reduction of 5 with H₂ under Pd/C catalytic conditions provides 7. Compound 5 is converted to 6 by Suzuki or Stille coupling, or by other nucleophilic substitution.

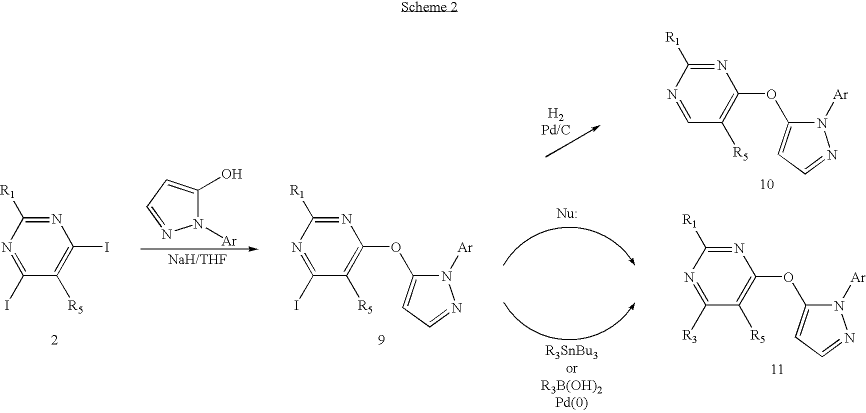

Scheme 2 illustrates the synthesis of compounds of formulas 10 and 11. Compound 8 is prepared essentially as described in Tetrahedron Lett. (1999) 40:1405-1408 or Eur. J. Med. Chem. Chim. Ther. (1989) 24:435-446. 8 is treated with NaH, followed by reaction with 2 to give 9. Reduction of 9 with H₂ under Pd/C catalytic conditions provides 10. 9 is converted to 11 by Suzuki or Stille coupling, or by other nucleophilic substitution.

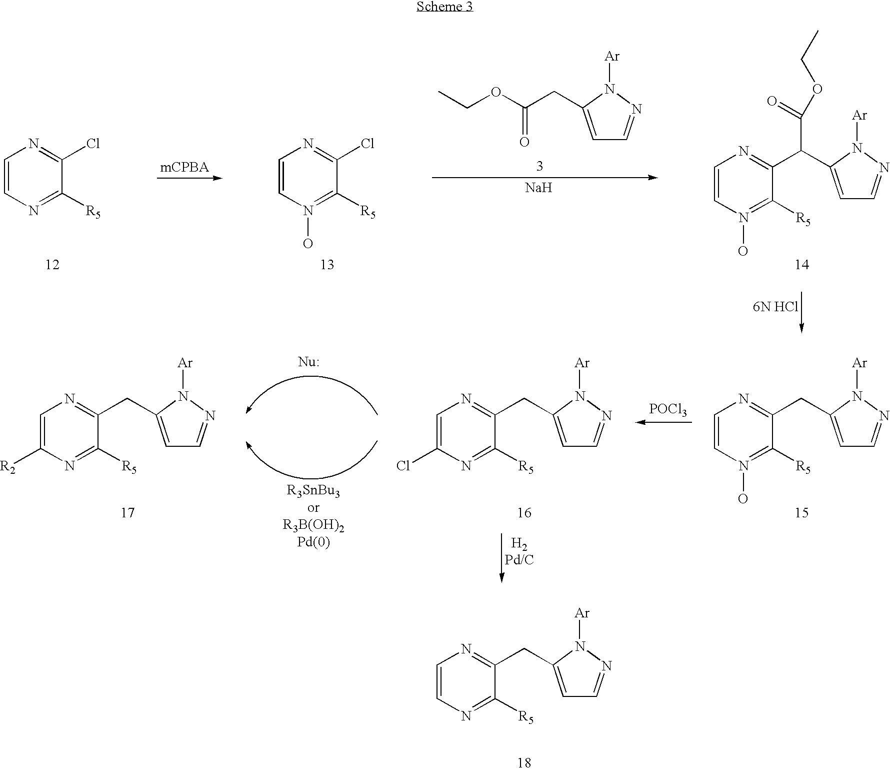

Scheme 3 illustrates the synthesis of pyrazines 17 and 18. Chloropyrazine 12 is prepared essentially as described in J. Am. Chem. Soc. (1952) 74:1580-1582. mCPBA treatment of 12 selectively oxidizes the nitrogen theta to the chlorine to provide 13. 13 reacts with 3 in the presence of NaH to produce 14, which is hydrolyzed and decarboxylated with 6 N HCl to give 15. 15 reacts with POCl₃ to afford chloride 16. Reduction of 16 with H₂ under Pd/C catalytic conditions provides 18. 16 is converted to 17 by Suzuki or Stille coupling, or by other nucleophilic substitution.

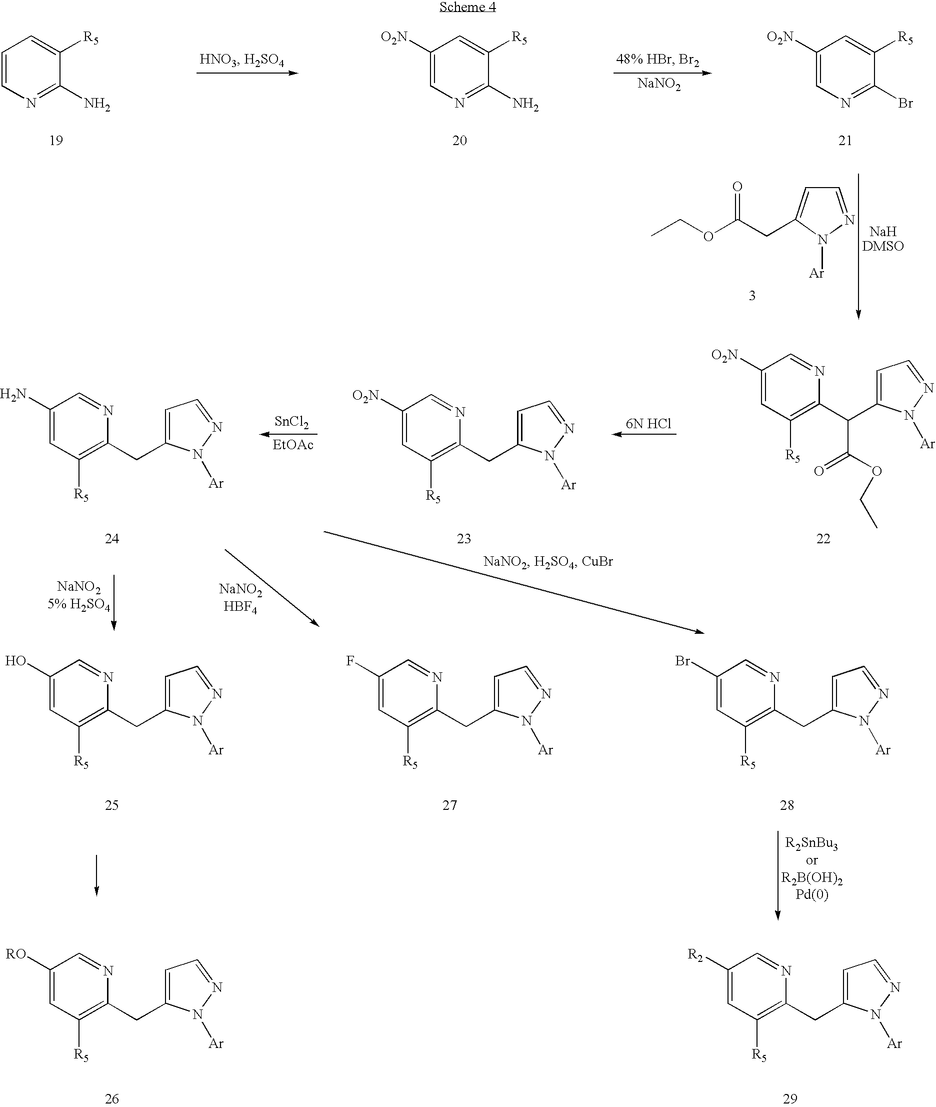

Scheme 4 illustrates the synthesis of pyridines 26, 27 and 29. Nitration of 19 gives 20, which is converted to 21 by diazotization to give the diazonium bromide and thermal decomposition thereof. Reaction of 21 with 3 in the presence of NaH gives 22. Hydrolysis and decarboxylation of 22 provides 23, which is reduced with SnCl₂ to give 24. Diazotization of 24 in 5% H₂SO₄ affords 25, which is reacted with alkyl halide to give 26. Compound 27 is prepared by diazotization of 24 to give the diazonium tetrafluoroborate and thermal decomposition. Diazotization of 24 in concentrated H₂SO₄ followed by treatment with CuBr provides 28. 28 is converted to compounds 29 by Suzuki or Stille coupling, or by other nucleophilic substitution.

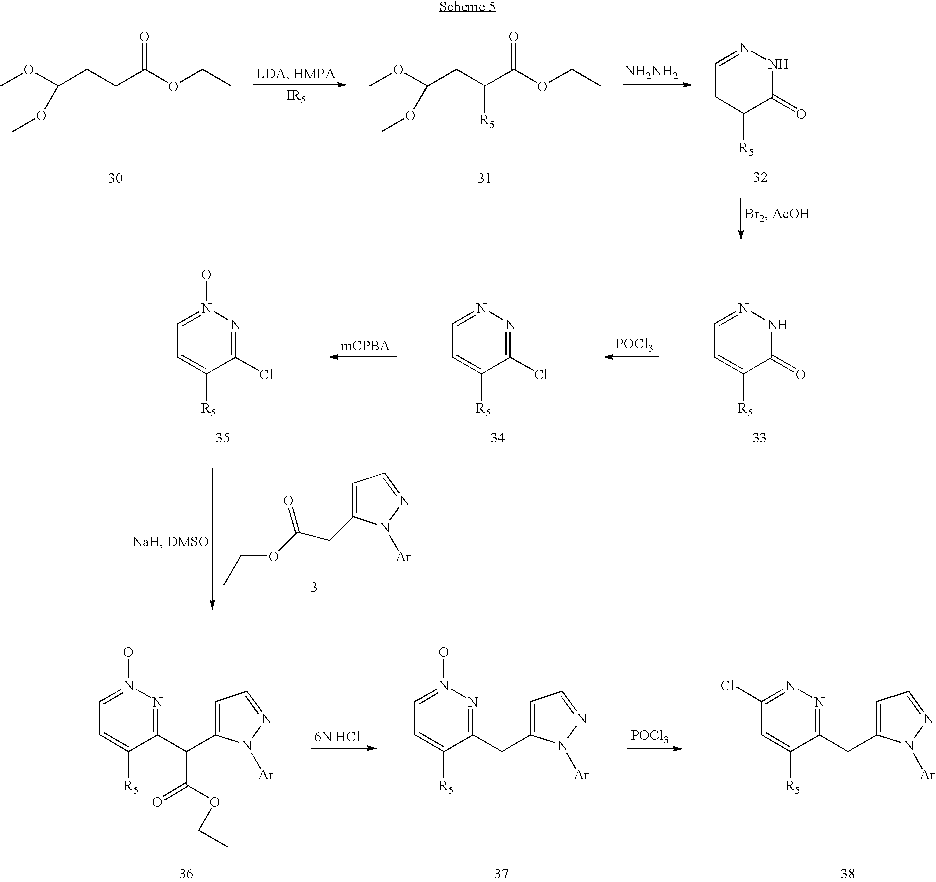

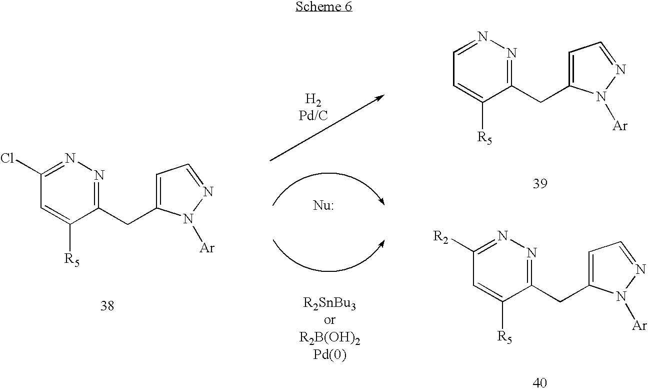

Scheme 5 and 6 illustrate the synthesis of intermediates 38, 39 and 40. Alkylation of 30 with an appropriate alkyl iodide gives 31, which reacts with hydrazine to afford 32. Aromatization of 32 by treatment with bromine in acetic acid provides pyridazinone 33, which is converted to chloropyridazine 34 upon treatment with POCl₃. N-oxidation of 34 with mCPBA affords N-oxide 35, which is reacted with 3 in the presence of NaH to give 36. Hydrolysis and then decarboxylation of 36 with 6 N HCl gives 37, which is reacted with POCl₃ to provide 38. Reduction of 38 with H₂ under Pd/C catalytic conditions provides 39. 38 is converted to 40 by Suzuki or Stille coupling, or by other nucleophilic substitution.

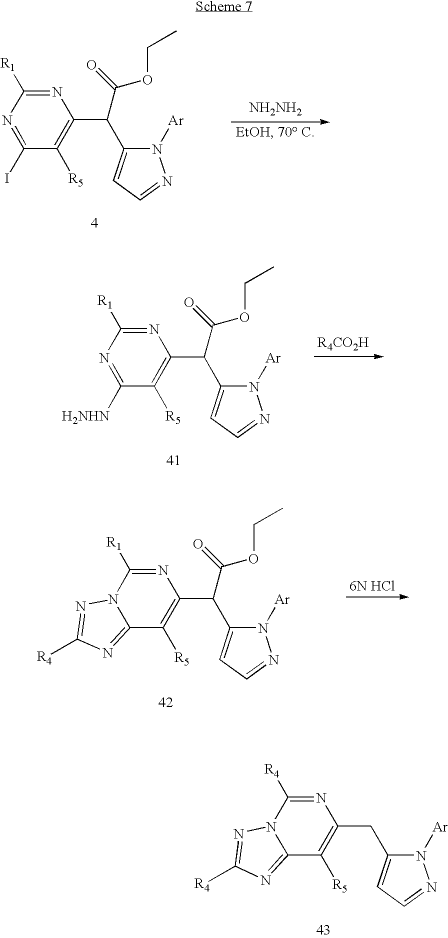

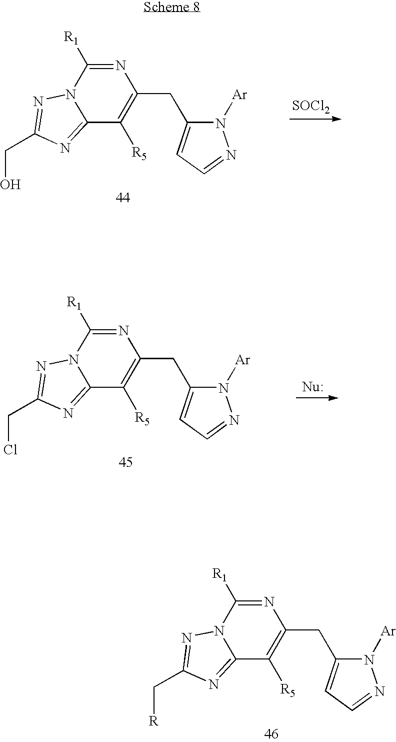

Schemes 7 and 8 illustrate the synthesis of triazole fused pyrimidines 43. Treatment of 4 with hydrazine gives intermediate 41, which upon refluxing with a carboxylic acid provides 42. Hydrolysis and decarboxylation of 42 with 6 N HCl affords 43. 44 is synthesized based on scheme 7 using hyroxylacetic acid. Treatment of 44 with thionyl chloride at room temperature gives intermediate chloride 45, which is converted to 46 by nucleophilic substitution.

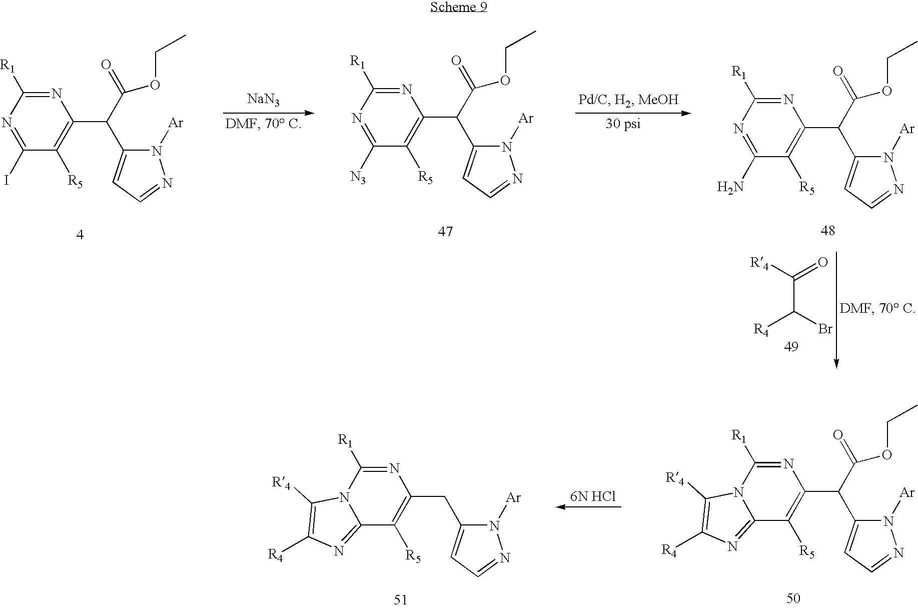

Scheme 9 illustrates the synthesis of compounds of formula 51 from 4. Treatment of 4 with NaN₃ in DMF at 70° C. overnight provides the corresponding 4-azido-pyrimidine compound 47, which can be converted to the amino-pyrimidine 48 by hydrogenation. Reaction of 50 with various α-bromo (or chloro) aldehydes or ketones 49 in DMF gives the desired imidazole fused pyrimidine 50, which is hydrolyzed and decarboxylated with 6 N HCl to provide 51.

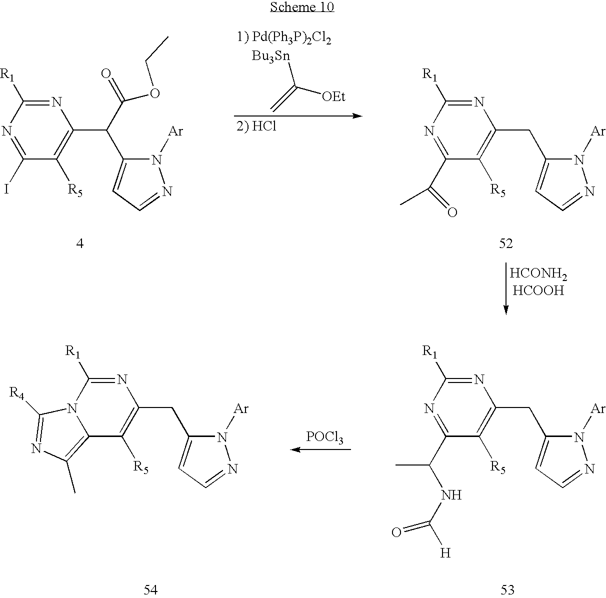

Scheme 10 illustrates the synthesis of imidazole fused pyrimidines 54. Intermediate 4 is coupled with tributyltinvinylethylether under Pd(Ph₃P)₂Cl₂ coupling conditions, followed by hydrolysis and decarboxylation to give the ketone 52. Treatment of ketone 52 with formamide and formic acid, followed by POCl₃ effected cyclization affords 54.

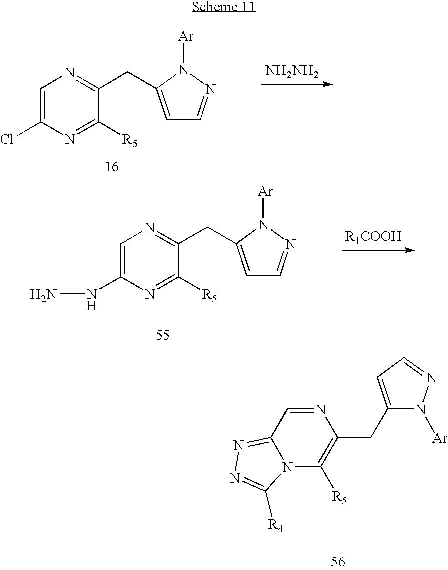

Scheme 11 illustrates the synthesis of triazole fused pyrazines 56. Treatment of 16 with hydrazine gives 55, which upon refluxing with a carboxylic acid provides 56.

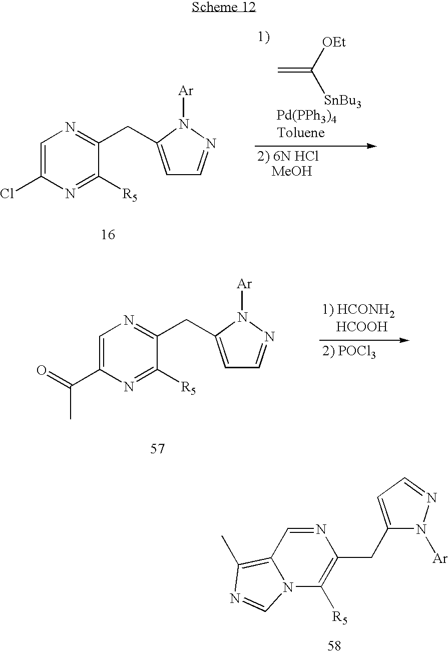

Scheme 12 illustrates the synthesis of imidazole fused pyrazines 58. 16 reacts with tributyltinvinylethylether in the presence of Pd(PPh₃)₄, followed by acid hydrolysis to afford 57. 57 reacts with formamide and formic acid, followed by POCl₃ to give 58.

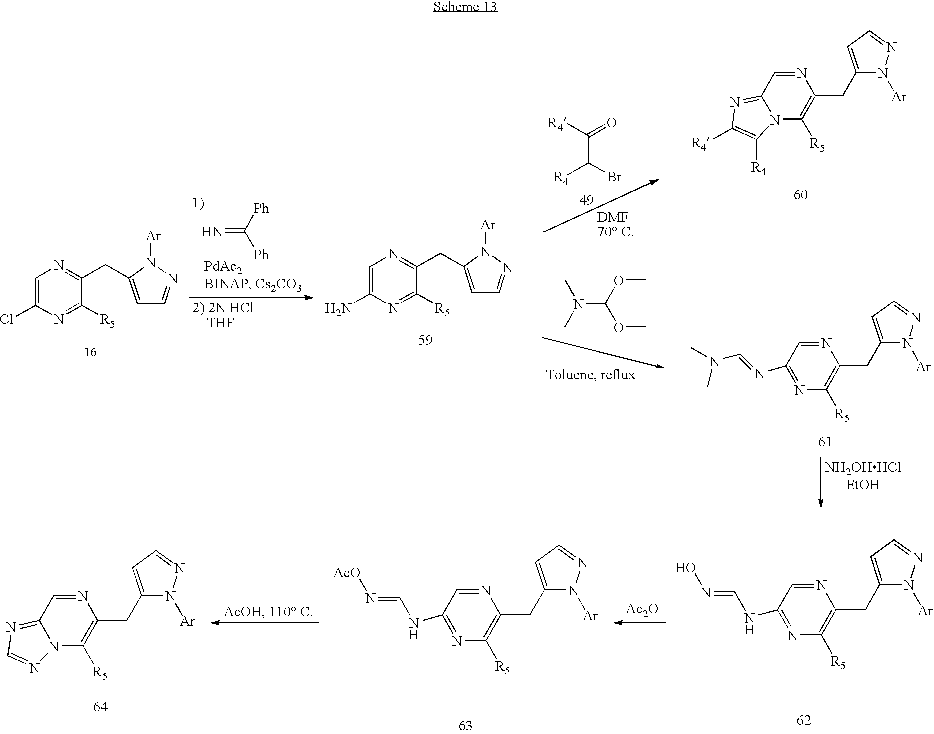

Scheme 13 illustrates the synthesis of imidazole fused pyrazines 60 and triazole fused pyrazines 64. Amination of 16 under Pd coupling conditions followed by acid cleavage provides 59, which condenses with various α-bromo (or chloro) aldehydes or ketones 49 in DMF to afford 60. Treatment of 59 with N,N-dimethylformamide dimethylacetal gives 61, which is reacted with hydroxylamine to give 62. Acetylation of 62 with acetic anhydride provides 63, which is heated with acetic acid to afford 64.

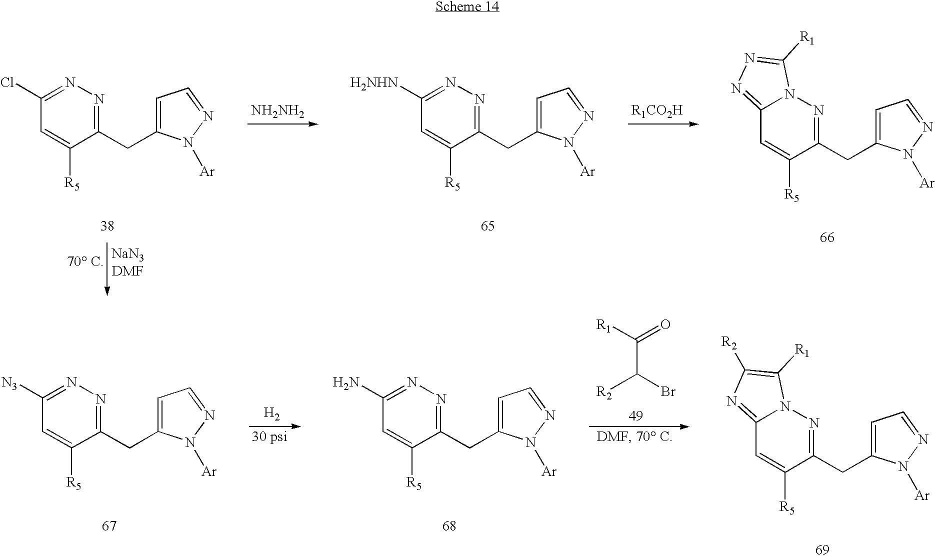

Scheme 14 illustrates the synthesis of triazole fused pyridazines 66 and imidazole fused pyridazines 69. Treatment of compound 38 with hydrazine gives 65, which upon refluxing with a carboxylic acid provides 66. Treatment of 38 with NaN₃ in DMF at 70° C. overnight provides the corresponding 4-azido-pyridazine compound 67, which can be converted to the amino-pyridazine 68 by hydrogenation. Reaction of compound 68 with various α-bromo (or chloro) aldehydes or ketones 49 in DMF furnishes 69.

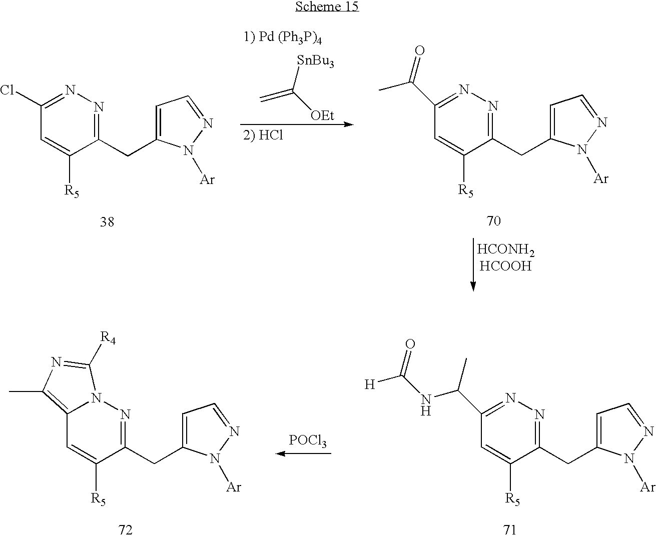

Scheme 15 illustrates the synthesis of imidazole fused pyridazines 72. Intermediate 38 is coupled with tributyltinvinylethylether under Pd(Ph₃P)₂Cl₂ coupling conditions, followed by hydrolysis to give ketone 70. Treatment of 70 with formamide and formic acid, followed by POCl₃ effects cyclization to afford 72.

Compounds may be radiolabeled by carrying out their synthesis using precursors comprising at least one atom that is a radioisotope. Each radioisotope is preferably carbon (e.g., ¹⁴C), hydrogen (e.g., ³H), sulfur (e.g., ³⁵S) or iodine (e.g., ¹²⁵I). Tritium labeled compounds may also be prepared catalytically via platinum-catalyzed exchange in tritiated acetic acid, acid-catalyzed exchange in tritiated trifluoroacetic acid, or heterogeneous-catalyzed exchange with tritium gas using the compound as substrate. In addition, certain precursors may be subjected to tritium-halogen exchange with tritium gas, tritium gas reduction of unsaturated bonds, or reduction using sodium borotritide, as appropriate. Preparation of radiolabeled compounds may be conveniently performed by a radioisotope supplier specializing in custom synthesis of radiolabeled probe compounds.

The following Examples are offered by way of illustration and not by way of limitation. Unless otherwise specified, all reagents and solvents are of standard commercial grade and are used without further purification. Starting materials and intermediates described herein may generally be obtained from commercial sources, prepared from commercially available organic compounds or prepared using well known synthetic methods.

### EXAMPLES

Starting materials and various intermediates described in the following Examples may be obtained from commercial sources, prepared from commercially available organic compounds, or prepared using known synthetic methods. Representative examples of methods suitable for preparing intermediates are also set forth below.

In the following Examples, LC-MS conditions for the characterization of the compounds herein are:
1. Analytical HPLC/MS instrumentation: Analyses are performed using a Waters 600 series pump (Waters Corp., Milford, Mass.), a Waters 996 Diode Array Detector and a Gilson 215 auto-sampler (Gilson Inc., Middleton, Wis.), Micromass® LCT time-of-flight electrospray ionization mass analyzer. Data are acquired using MassLynx™ 4.0 software, with OpenLynx Global Server™, OpenLynx™ and AutoLynx™ processing.
2. Analytical HPLC conditions: 4.6×50 mm, Chromolith™ SpeedROD RP-18e column (Merck KGaA, Darmstadt, Germany); UV 10 spectra/sec, 220-340 nm summed; flow rate 6.0 mL/min; injection volume 1 μL;
   Gradient conditions—mobile phase A is 95% water, 5% MeOH with 0.05% TFA; mobile phase B is 95% MeOH, 5% water with 0.025% TFA, and the gradient is 0-0.5 minutes 10-100% B, hold at 100% B to 1.2 minutes, return to 10% B at 1.21 minutes inject-to-inject cycle time is 2.15 minutes.
3. Analytical MS conditions: capillary voltage 3.5 kV; cone voltage 30V; desolvation and source temperature are 350° C. and 120° C., respectively; mass range 181-750 with a scan time of 0.22 seconds and an inter scan delay of 0.05 minutes.

All compounds of Formula I shown in Examples 1-7, including the Tables, exhibit a Kᵢ of less than 1 micromolar in the ligand binding assay provided in Example 8.

### Example 1

### Synthesis of Pyrimidines

### A. 4-{[1-(3-FLUOROPYRIDIN-2-YL)-1H-PYRAZOL-5-YL]METHYL}-5-PROPYLPYRIMIDINE (106)

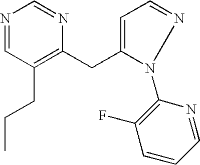

### Step 1. 4,6-Dihydroxy-5-propylpyrimidine (100)

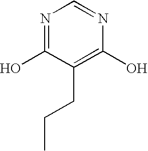

This compound is prepared essentially as described in J. Heterocycl. Chem. (1974) 11:295-297. Propyl diethylmalonate (50.6 g, 0.25 mol) is added to a solution of sodium ethoxide (34.02 g, 0.5 mol) in EtOH (300 mL). This is followed by addition of formamidine acetate (26.03 g, 0.25 mol).

The reaction is stirred overnight, and solvent is removed. The residue is dissolved in water and acidified with acetic acid to form a white precipitate, which is filtered, washed with water and then EtOH, and dried to give the product 100, which is used in the next step without further purification.

### Step 2. 4,6-dichloro-5-propylpyrimidine (101)

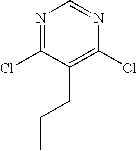

This compound is prepared essentially as described in J. Heterocycl. Chem. (1974) 11:295-297. A mixture of 100 (37 g, 0.24 mol) and phosphoryl chloride (200 mL) is refluxed for 1.5 hours. Excess POCl₃ is removed, and the residue is poured with stirring onto ice. The mixture is neutralized with saturated NaHCO₃. This is followed by extraction with DCM. The solvent is evaporated to give compound 101. ¹H NMR δ (CDCl₃) 1.05 (t, 3H), 1.61-1.71 (m, 2H), 2.68 (s, 3H), 8.61 (s, 1H).

### Step 3. 4,6-diiodo-5-propylpyrimidine (102)

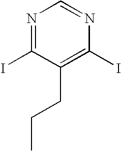

A solution of 101 (8.85 g, 46.6 mmol) in acetone (225 mL) is treated with NaI (34.8 g, 231 mmol) and HI (57% solution, 36 mL) at ambient temperature overnight. The resulting mixture is poured into a beaker with ice water, filtered, and air-dried to give the product 102. ¹H NMR δ (CDCl₃) 1.09 (t, 3H), 1.61-1.67 (m, 2H), 2.90 (s, 3H), 8.21 (s, 1H).

### Step 4. [2-(3-fluoro-pyridin-2-yl)-2H-pyrazol-3-yl]-acetic acid ethyl ester (103)

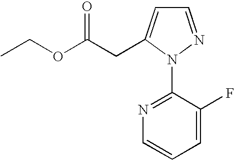

This compound is prepared essentially as described in Chem. Ber. (1985) 118:741-752 and Farmaco (1990) 45:167-186. A mixture of 3,5-diethoxy-penta-2,4-dienoic acid ethyl ester (2.4 g, 11.2 mmol) and (3-fluoro-pyridin-2-yl)hydrazine (1.4 g, 11.0 mmol) in EtOH (30 mL) and concentrated HCl (6 mL) is heated at 90° C. for 2 hours. The solvent is removed, and the residue is neutralized with saturated NaHCO₃ and extracted with DCM. The solution is dried and evaporated, and the residue is column purified (EtOAc:hexane=2:1) to give 103. ¹H NMR δ (CDCl₃) 1.13 (t, 3H), 3.97 (s, 2H), 4.04 (q, 2H), 6.42 (s, 1H), 7.30-7.38 (m, 1H), 7.64 (td, 1H), 7.72 (s, 1H), 8.28 (d, 1H).

### Step 5. [2-(3-fluoro-pyridin-2-yl)-2H-pyrazol-3-yl]-(6-iodo-5-propyl-pyrimidin-4-yl)-acetic acid ethyl ester (104)

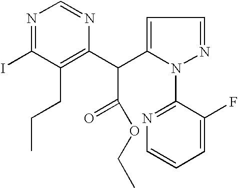

NaH (0.29 g, 60% in mineral oil, 7.2 mmol) is added to a solution of [2-(3-fluoro-pyridin-2-yl)-2H-pyrazol-3-yl]-acetic acid ethyl ester (103) (1.5 g, 6.02 mmol) in DMSO (10 mL) at room temperature. The mixture is stirred for 10 minutes and then 4,6-diiodo-5-propylpyrimidine (102) (2.25 g, 6.02 mmol) is added. The resulting mixture is heated at 60° C. for two hours. The mixture is cooled to room temperature, and water (20 mL) is added, followed by extraction with ethyl acetate (3×50 mL). The solution is dried and evaporated. The residue is column purified with 2% MeOH in DCM to give the product 104. ¹H NMR δ (CDCl₃) 0.97 (t, 3H), 1.11 (t, 3H), 1.43-1.52 (m, 2H), 2.64 (t, 2H), 6.42 (s, 1H), 4.11 (q, 2H), 6.08 (s, 1H), 6.11 (s, 1H), 7.35-7.42 (m, 1H), 7.64-7.72 (m, 2H), 8.29 (d, 1H), 8.63 (s, 1H). LC-MS M+1 495.90.

### Step 6. [2-(3-fluoro-pyridin-2-yl)-2H-pyrazol-3-yl]-(5-propyl-pyrimidin-4-yl)-acetic acid ethyl ester (105)

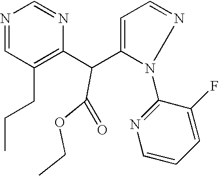

Pd/C (10%, 10 mg) is added to a solution of [2-(3-fluoro-pyridin-2-yl)-2H-pyrazol-3-yl]-(6-iodo-5-propyl-pyrimidin-4-yl)-acetic acid ethyl ester (104) (80 mg) in EtOH (10 mL). The mixture is stirred under H₂ overnight. The catalyst is removed by filtration and the filtrate is evaporated in vacuo to give 105.

### Step 7. 4-{[1-(3-fluoropyridin-2-yl)-1H-pyrazol-5-yl]methyl}-5-propylpyrimidine (106)

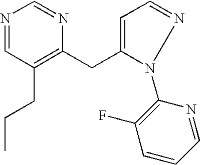

A mixture of [2-(3-fluoro-pyridin-2-yl)-2H-pyrazol-3-yl]-(5-propyl-pyrimidin-4-yl)-acetic acid ethyl ester (105) from above reaction in 6N HCl (10 mL) is heated at 60° C. for 4 hours. The reaction mixture is cooled to room temperature and is neutralized with saturated NaHCO₃. This is followed by extraction with DCM. The solution is dried and evaporated. The residue is purified by column with 5% MeOH in DCM to give the product 106. ¹H NMR δ (CDCl₃) 0.94 (t, 3H), 1.51-1.58 (m, 2H), 2.53 (t, 2H), 4.49 (s, 2H), 6.15 (s, 1H), 7.30-7.39 (m, 1H), 7.60 (td, 2H), 7.70 (s, 1H), 8.27 (m, 1H), 8.42 (m, 1H), 8.86 (s, 1H).

### B. 4-{[1-(3-FLUOROPYRIDIN-2-YL)-1H-PYRAZOL-5-YL]METHYL}-6-METHOXY-5-PROPYLPYRIMIDINE (108)

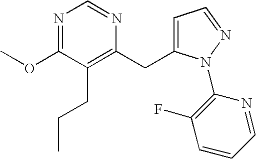

### Step 1. 4-chloro-6-[2-(3-fluoro-pyridin-2-yl)-2H-pyrazol-3-ylmethyl]-5-propyl-pyrimidine (107)

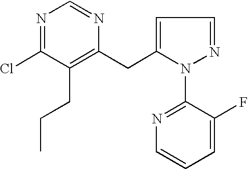

A mixture of [2-(3-fluoro-pyridin-2-yl)-2H-pyrazol-3-yl]-(6-iodo-5-propyl-pyrimidin-4-yl)acetic acid ethyl ester (104) (0.5 g, 1.0 mmol) in 6N HCl (10 mL) is stirred at room temperature overnight. The reaction mixture is neutralized with saturated NaHCO₃. This is followed by extraction with DCM. The solution is dried and evaporated, and the residue is purified by column with 5% MeOH in DCM to give the product 107.

### Step 2. 4-{[1-(3-fluoropyridin-2-yl)-1H-pyrazol-5-yl]methyl}-6-methoxy-5-propylpyrimidine (108)

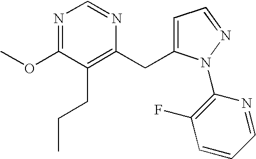

To a solution of 4-chloro-6-[2-(3-fluoro-pyridin-2-yl)-2H-pyrazol-3-ylmethyl]-5-propylpyrimidine (107) (15 mg) in MeOH (10 mL), NaOMe (25% solution in MeOH, 0.1 mL) is added. The mixture is stirred at room temperature for 2 hours and is quenched with water (1 mL). The mixture is extracted with DCM, dried and evaporated. The residue is purified by TLC with 5% MeOH in DCM to give 108. ¹H NMR δ (CDCl₃) 0.89 (t, 3H), 1.3.7-1.47 (m, 2H), 2.48 (t, 2H), 3.95 (s, 3H), 4.31 (s, 2H), 6.15 (s, 1H), 7.33-7.39 (m, 1H), 7.58-7.80 (m, 2H), 8.31 (m, 1H), 8.46 (s, 1H).

### C. N-ethyl-6-{[1-(3-fluoropyridin-2-yl)-1H-pyrazol-5-yl]methyl}-5-propylpyrimidin-4-amine (109)

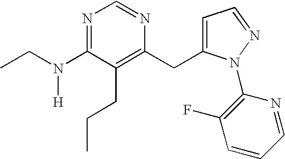

A mixture of 4-chloro-6-[2-(3-fluoro-pyridin-2-yl)-2H-pyrazol-3-ylmethyl]-5-propylpyrimidine (107) (150 mg) and ethylamine (1 mL, 2 m solution in EtOH) in EtOH (5 mL) is heated at 110° C. overnight. The solvent is removed and the residue is treated with water. The mixture is extracted with DCM, dried and evaporated. The residue is TLC purified with 5% MeOH in DCM to give 109. ¹H NMR δ (CDCl₃) 0.93 (t, 3H), 1.24 (t, 3H), 1.38-1.50 (m, 2H), 2.32 (t, 2H), 3.45-3.55 (m, 2H), 4.19 (s, 2H), 4.58 (s, 1H), 6.11 (s, 1H), 7.31-7.37 (m, 1H), 7.60-7.67 (m, 2H), 8.34-8.40 (m, 2H).

### D. 2-[5-(6-ETHOXY-5-PROPYL-PYRIMIDIN-4-YLMETHYL)-PYRAZOL-1-YL]-NICOTINONITRILE (113)

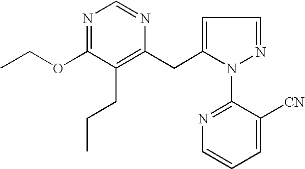

### Step 1. [2-(3-bromo-pyridin-2-yl)-2H-pyrazol-3-yl]-(6-iodo-5-propyl-pyrimidin-4-yl)-acetic acid ethyl ester (110)

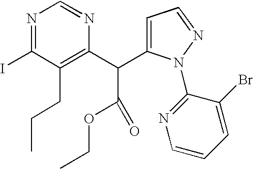

A solution of [2-(3-bromo-pyridin-2-yl)-2H-pyrazol-3-yl]-acetic acid ethyl ester (prepared as described in example 1A (step 4)) (3.38 g, 10.9 mmol) in 5 mL of anhydrous DMSO is added to a suspension of sodium hydride (60%, 435 mg, 13.1 mmol, 1.2 eq.) in 10 mL of DMSO dropwise at 5° C. under N₂. The resulting mixture is stirred at room temperature for 20 minutes, and then a solution of 4,6-diiodo-5-propylpyrimidine (4.29 g, 10.9 mmol, 1.0 eq.) in 5 mL of DMSO is added, and the reaction mixture is heated to 60° C. and stirred for 3 hours. The reaction mixture is cooled to room temperature, quenched with aqueous ammonium chloride solution, extracted with EtOAc (50 mL×3), washed with water and brine, dried over Na₂SO₄, concentrated and purified via silica gel chromatography (hexanes/EtOAc, from 8:1 to 2:1) to give 110.

### Step 2. 6-[2-(3-bromo-pyridin-2-yl)-2H-pyrazol-3-ylmethyl]-5-propyl-3H-pyrimidin-4-one (111)

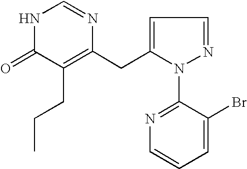

To a solution of the [2-(3-bromo-pyridin-2-yl)-2H-pyrazol-3-yl]-(6-iodo-5-propyl-pyrimidin-4-yl)-acetic acid ethyl ester (110) (1.8 g, 3.24 mmol) in 15 mL of THF is added 40 mL 6 N HCl. The reaction mixture is stirred at 60° C. for 5 hours. The solvent is removed under reduced pressure. After adjusting the pH to 8.0 with NaHCO₃, the resulting mixture is extracted with CH₂Cl₂ (4×100 mL). The combined organic layers are washed with 20 mL of brine, dried over MgSO₄ and concentrated under reduced pressure. The crude product is washed with ether to give the title compound III as a white solid. ¹H NMR δ (CDCl₃) 8.51 (dd, 1H), 8.08 (dd, 1H), 7.89 (s, 1H), 7.68 (d, 1H), 7.28 (dd, 1H), 6.24 (d, 1H), 4.04 (s, 2H), 2.32-2.37 (m, 2H), 1.36-1.46 (m, 2H), 0.91 (t, 3H).

### Step 3. 2-[5-(6-Oxo-5-propyl-1,6-dihydro-pyrimidin-4-ylmethyl)-pyrazol-1-yl]-nicotinonitrile (112)

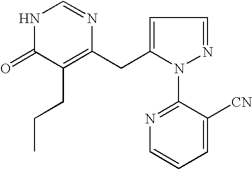

To a solution of 6-[2-(3-bromo-pyridin-2-yl)-2H-pyrazol-3-ylmethyl]-5-propyl-3H-pyrimidin-4-one (111) (890 mg, 2.38 mmol) in 10 mL of anhydrous DMF is added Zn(CN)₂ (195 mg, 1.67 mmol), Pd₂(dba)₃ (109 mg, 0.189 mmol) and DPPF (127 mg, 0.238 mmol). The reaction mixture is stirred at 110° C. overnight. The solvent is removed under reduced pressure, and then 10 mL NaHCO₃ is added. The resulting mixture is extracted with CH₂Cl₂ (3×50 mL). The combined organic layers are washed with 20 mL of brine, dried over MgSO₄ and concentrated under reduced pressure. The resulting residue is purified by flash column chromatography eluted with 3:1 EtOAc and hexanes to give the title compound 112. ¹H NMR δ (CDCl₃) 8.58 (dd, 1H), 8.15 (dd, 1H), 7.90 (s, 1H), 7.75 (d, 1H), 7.34 (dd, 1H), 6.24 (d, 1H), 4.44 (s, 2H), 2.50-2.56 (m, 2H), 1.50-1.57 (m, 2H), 0.98 (t, 3H).

### Step 4. 2-[5-(6-Ethoxy-5-propyl-pyrimidin-4-ylmethyl)-pyrazol-1-yl]-nicotinonitrile (113)

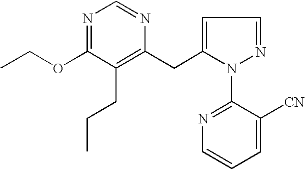

To a solution of 2-[5-(6-oxo-5-propyl-1,6-dihydro-pyrimidin-4-ylmethyl)-pyrazol-1-yl]-nicotinonitrile (112) (32 mg, 0.10 mmol) in 1 mL of anhydrous DMF is added ethyl iodide (47 mg, 0.30 mmol) and K₂CO₃ (41 mg, 0.30 mmol). The reaction mixture is stirred at room temperature overnight. The solvent is removed under reduced pressure. The resulting residue is diluted with 50 mL of EtOAc, washed with 10 mL of water and 10 mL of brine, dried over MgSO₄ and concentrated under reduced pressure. The residue is purified by preparative TLC to give the titled compound 113. ¹H NMR δ (CDCl₃) 8.54 (dd, 1H), 8.42 (s, 1H), 8.14 (dd, 1H), 7.73 (d, 1H), 7.31 (dd, 1H), 6.14 (d, 1H), 4.54 (s, 2H), 4.40 (q, 2H), 2.54-2.59 (m, 2H), 1.47-1.55 (m, 2H), 1.41 (t, 3H), 0.94 (t, 3H).

Compounds 114 and 115 shown in Table 1 are synthesized via similar procedures.

TABLE 1

| | Compound | Name | LC-MS/NMR |
|---|---|---|---|
| 114 | 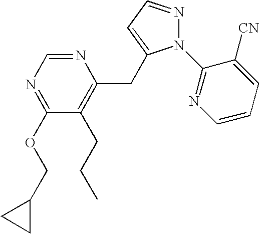 | 2-[5-(6- Cyclopropylmethoxy-5- propyl-pyrimidin-4- ylmethyl)-pyrazol-1-yl]- nicotinonitrile | ¹H NMR δ (CDCl₃) 8.55 (dd, 1H), 8.40 (s, 1H), 8.14 (dd, 1H), 7.73 (d, 1H), 7.32 (dd, 1H), 6.14 (d, 1H), 4.54 (s, 2H), 4.18 (d, 2H), 2.56-2.61 (m, 2H), 1.50- 1.57 (m, 2H), 1.20-1.30 (m, 1H), 0.95 (t, 3H), 0.56-0.63 (m, 2H), |
| #0.32-0.37 (m, 2H) | | | |
| 115 | 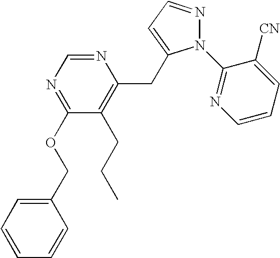 | 2-[5-(6-Benzyloxy-5-propyl- pyrimidin-4-ylmethyl)- pyrazol-1-yl]-nicotinonitrile | ¹H NMR δ (CDCl₃) 8.53 (dd, 1H), 8.45 (s, 1H), 8.13 (dd, 1H), 7.77 (d, 1H6), 7.35-7.43 (m, 5H), 7.30 (dd, 1H), 6.15 (d, 1H), 5.42 (s, 2H), 4.56 (s, 2H), 2.59-2.64 (m, 2H), 1.50-1.57 (m, 2H), 0.94 (t, 3H) |

### E. 4-{[1-(6-FLUOROPYRIDIN-2-YL)-1H-PYRAZOL-5-YL]OXY}-5-PROPYLPYRIMIDINE (117)

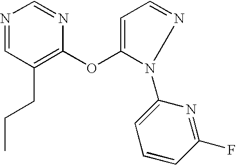

### Step 1. 4-{[1-(6-fluoropyridin-2-yl)-1H-pyrazol-5-yl]oxy}-6-iodo-5-propylpyrimidine (116)

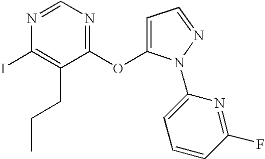

To a solution of 2-(6-fluoro-pyridin-2-yl)-2H-pyrazol-3-ol (60 mg) in DMSO is added NaH (16 mg, 60% in mineral oil). The mixture is stirred at room temperature for 10 minutes. 4,6-Diiodo-5-propylpyrimidine (125 mg) is added and the resulting mixture is stirred at 60° C. overnight. Water (10 mL) and EtOAc (10 mL) are added to the mixture. The layers are separated and the aqueous layer is extracted with EtOAc (2×10 mL). The combined extracts are washed with brine (15 mL), dried with Na₂SO₄, filtered and evaporated in vacuo. PTLC in 5% MeOH/DCM gives 116. ¹H NMR δ (CDCl₃) 1.08 (t, 3H), 1.71-1.80 (m, 2H), 2.88 (t, 2H), 6.26 (d, 1H), 6.70-6.74 (m, 1H), 7.72-7.90 (m, 3H), 8.16 (s, 1H).

### Step 2. 4-{[1-(6-fluoropyridin-2-yl)-1H-pyrazol-5-yl]oxy}-5-propylpyrimidine (117)

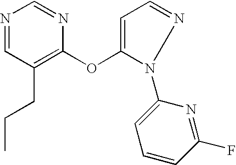

Pd/C (10%, 10 mg) is added to a solution of 4-{[1-(6-fluoropyridin-2-yl)-1H-pyrazol-5-yl]oxy}-6-iodo-5-propylpyrimidine (116) (20 mg) in EtOH (10 mL). The mixture is stirred under H₂ overnight. The catalyst is removed by filtration and the filtrate is evaporated in vacuo. PTLC with 5% MeOH/DCM gives the pure product (117). ¹H NMR δ (CDCl₃) 1.03 (t, 3H), 1.73-1.86 (m, 2H), 2.77 (t, 2H), 6.28 (d, 1H), 6.68-6.72 (m, 1H), 7.72-7.90 (m, 3H), 8.48 (s, 1H), 8.54 (s, 1H).

### F. 2-[5-(6-METHYL-5-PROPYL-PYRIMIDIN-4-YLMETHYL)-PYRAZOL-1-YL]-NICOTINONITRILE (122)

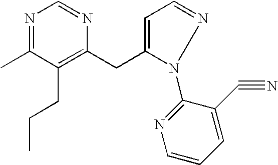

### Step 1. 4-Iodo-6-methyl-5-propylpyrimidine (119)

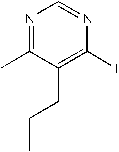

To a solution of 4-chloro-6-methyl-5-propylpyrimidine (118) (5.85 g, 34.3 mmol) in acetone (100 mL) is added NaI (12.75 g, 85 mL) in several portions. The resulting suspension is stirred at ambient temperature for 30 minutes. HI (57%, 13 mL) is added dropwise and the orange solution is stirred at ambient temperature for 3 hours. The solvent is evaporated in vacuo, and water (50 mL) and EtOAc (80 mL) are added. The layers are separated and the aqueous layer is extracted with EtOAc (80 mL). The combined extracts are washed with water (50 mL), saturated NaHCO₃ (50 mL) and brine (50 mL), dried (Na₂SO₄) and evaporated in vacuo. The residue is purified with flash column chromatography (EtOAc:hexanes=8:1) to provide the title compound 119.

### Step 2. [1-(3-chloropyridin-2-yl)-1H-pyrazol-5-yl](6-methyl-5-propylpyrimidin-4-yl)acetate (120)

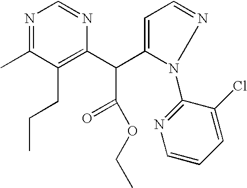

To a solution of ethyl [1-(3-chloropyridin-2-yl)-1H-pyrazol-5-yl]acetate (660 mg, 2.48 mmol) in DMSO (4 mL) is added NaH (60% in mineral oil, 120 mg, 3 mmol) in several portions. The resulting red solution is stirred at ambient temperature for 30 minutes. 4-Iodo-6-methyl-5-propylpyrimidine (119) (328 mg, 1.25 mmol) is added and the mixture is heated at 95° C. for 2 hours. Water (20 mL) is added and the mixture is extracted with EtOAc (2×30 mL). The combined extracts are washed with brine, dried (Na₂SO₄) and evaporated. The residue is purified with flash column chromatography (EtOAc:hexane=1:1) to provide the title compound 120.

### Step 3. 4-{[1-(3-chloropyridin-2-yl)-1H-pyrazol-5-yl]methyl}-6-methyl-5-propylpyrimidine (121)

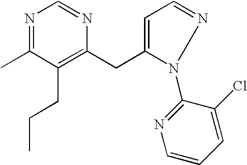

A solution of ethyl [1-(3-chloropyridin-2-yl)-1H-pyrazol-5-yl](6-methyl-5-propylpyrimidin-4-yl)acetate (120) (212 mg, 0.53 mmol) in HCl (6N, 5 mL) is stirred at 60° C. for 3 hours. The solution is cooled, basified with solid NaHCO₃ and extracted with EtOAc (2×10 mL). The combined extracts are washed with brine, dried (Na₂SO₄) and evaporated. The residue is purified with PTLC with 3% MeOH in CH₂Cl₂, providing the title compound 121. ¹H NMR δ (CDCl3) 8.70 (s, 1H), 8.43 (dd, 1H), 7.88 (dd, 1H), 7.67 (d, 1H), 7.33 (dd, 1H), 6.13 (m, 1H), 4.22 (s, 2H), 2.48-2.52 (m, 2H), 2,47 (s, 3H), 1.36-1.42 (m, 2H), 0.95 (t, 3H).

### Step 4. 2-{5-[(6-methyl-5-propylpyrimidin-4-yl)methyl]-1H-pyrazol-1-yl}nicotinonitrile (122)

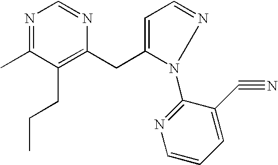

A solution of 4-{[1-(3-chloropyridin-2-yl)-1H-pyrazol-5-yl]methyl}-6-methyl-5-propylpyrimidine (121) (85 mg, 0.26 mmol), Zn(CN)₂ (22 mg, 0.182 mmol), Pd(dba)₃ (12 mg, 0.013 mmol) and DPPF (14 mg, 0.026 mmol) in DMF (3 mL) is degassed by argon for 10 minutes. The dark red solution is heated in a sealed tube at 130° C. overnight. The solvent is removed in vacuo, and water (4 mL) and EtOAc (10 mL) are added. The combined extracts are washed with brine (7 mL), dried (Na₂SO₄) and evaporated. The residue is PTLC purified with 3% MeOH in CH₂Cl₂ to provide 122. ¹H NMR δ (CDCl₃) 8.85 (s, 1H), 8.56 (dd, 1H), 8.42 (d, 1H), 8.09 (dd, 1H), 7.25 (dd, 1H), 6.42 (d, 1H), 4.27 (s, 2H), 2.78-2.83 (m, 2H), 2,53 (s, 3H), 1.49-1.58 (m, 2H), 1.05 (t, 3H).

### Example 2

### Synthesis of [1,2,4]TRIAZOLO[1,5-C]PYRIMIDINES

### A. 7-{[1-(3-FLUOROPYRIDIN-2-YL)-1H-PYRAZOL-5-YL]METHYL}-2-METHYL-8-PROPYL[1,2,4]TRIAZOLO[1,5-c]PYRIMIDINE (125)

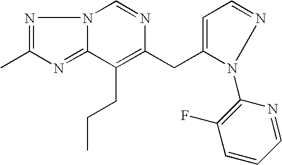

### Step 1. [2-(3-fluoro-pyridin-2-yl)-2H-pyrazol-3-yl]-(6-hydrazino-5-propyl-pyrimidin-4-yl)-acetic acid ethyl ester (123)

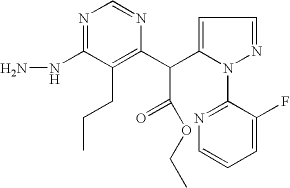

A mixture of [2-(3-fluoro-pyridin-2-yl)-2H-pyrazol-3-yl]-(6-iodo-5-propyl-pyrimidin-4-yl)acetic acid ethyl ester (104) (1 g, 2 mmol) with anhydrous hydrazine (0.3 g, 9 mmol) in EtOH is heated at 70° C. overnight. The solvent is removed to yield solid 123, which is used for the next step without purification.

### Step 2. Ethyl [1-(3-fluoropyridin-2-yl)-1H-pyrazol-5-yl](2-methyl-8-propyl[1,2,4]triazolo[1,5-c]pyrimidin-7-yl)acetate (124)

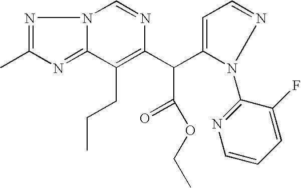

A mixture of the crude [2-(3-fluoro-pyridin-2-yl)-2H-pyrazol-3-yl]-(6-hydrazino-5-propylpyrimidin-4-yl)-acetic acid ethyl ester (123) in acetic acid (20 mL) is heated at 110° C. for 6 hours. The solvent is removed, and the residue is neutralized with saturated NaHCO₃. This is followed by extraction with DCM. The solvent is removed and the residue is purified by column with 5% MeOH in DCM to give the product 124. ¹H NMR δ (CDCl₃) 0.94 (t, 3H), 1.08 (t, 3H), 1.58-1.67 (m, 2H), 2.61 (s, 3H), 2.80-3.00 (m, 2H), 4.08 (q, 2H), 6.12 (s, 1H), 6.24 (s, 1H), 7.33-7.38 (m, 1H), 7.63-7.69 (m, 2H), 8.31 (dd, 1H), 7.85 (t, 1H), 9.15 (s, 1H).

### Step 3. 7-{[1-(3-fluoropyridin-2-yl)-1H-pyrazol-5-yl]methyl}-2-methyl-8-propyl[1,2,4]triazolo[1,5-c]pyrimidine (125)

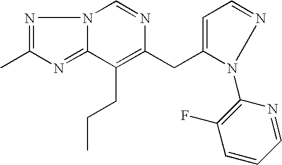

A mixture of ethyl [1-(3-fluoropyridin-2-yl)-1H-pyrazol-5-yl](2-methyl-8-propyl[1,2,4]triazolo[1,5-c]pyrimidin-7-yl)acetate (124) (0.5 g, 1.2 mmol) in 6N HCl (10 mL) is heated at 60° C. for 3 hours. The reaction mixture is cooled to room temperature and neutralized with saturated NaHCO₃. This is followed by extraction with DCM. The solution is dried and evaporated, and the residue is purified by column with 5% MeOH in DCM to give the product 125. ¹H NMR δ (CDCl₃) 0.97 (t, 3H), 1.60-1.71 (m, 2H), 2.60 (s, 3H), 2.87 (t, 2H), 4.42 (s, 2H), 6.15 (s, 1H), 7.33-7.39 (m, 1H), 7.62 (t, 1H), 7.69 (s, 1H), 8.33 (s, 1H), 8.99 (s, 1H).

### B. 2-(5-{[2-(DIFLUOROMETHYL)-8-PROPYL[1,2,4]TRIAZOLO[1,5-c]PYRIMIDIN-7-YL]METHYL}-1H-PYRAZOL-1-YL)NICOTINONITRILE (131)

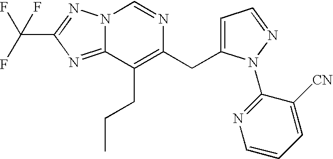 Step 1. [2-(3-bromo-pyridin-2-yl)-2H-pyrazol-3-yl]-(6-hydrazino-5-propyl-pyrimidin-4-yl)-acetic acid ethyl ester (127)

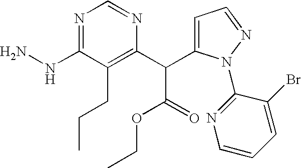

To a solution of [2-(3-bromo-pyridin-2-yl)-2H-pyrazol-3-yl]-(6-iodo-5-propyl-pyrimidin-4-yl)-acetic acid ethyl ester (110) (2.36 g, 4.24 mmol) in 30 mL of anhydrous EtOH is added anhydrous hydrazine (408 mg, 12.7 mmol, 3.0 eq.). The mixture is heated to 70° C. for 6 hours. EtOH is removed, the residue is diluted with 60 mL of DCM, washed with saturated aqueous NaHCO₃, dried over Na₂SO₄, and concentrated to give the product 127.

### Step 2. [2-(3-bromo-pyridin-2-yl)-2H-pyrazol-3-yl]-{5-propyl-6-[N′-(2,2,2-trifluoro-acetyl)hydrazino]-pyrimidin-4-yl}-acetic acid ethyl ester (128)

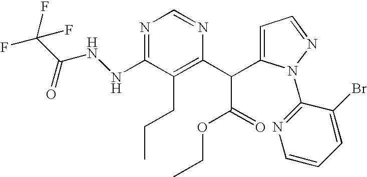

To a solution of [2-(3-bromo-pyridin-2-yl)-2H-pyrazol-3-yl]-(6-hydrazino-5-propylpyrimidin-4-yl)-acetic acid ethyl ester (127) (1.00 g, 2.17 mmol) in 20 mL of anhydrous DCM containing 2.0 equivalent of trimethylamine at 0° C. is added trifluoroacetic anhydride (547 mg, 2.6 mmol, 1.2 eq.) dropwise. The resulting mixture is stirred at room temperature for 6 hours. Water (10 mL) is added to quench the reaction, which is then basified with 10 mL of saturated aqueous NaHCO₃. The organic solution is collected, dried over Na₂SO₄, concentrated to give crude (128) as a sticky oil.

### Step 3. [2-(3-bromo-pyridin-2-yl)-2H-pyrazol-3-yl]-(8-propyl-2-trifluoromethyl-[1,2,4]triazolo[1,5-c]pyrimidin-7-yl)-acetic ethyl ester (129)

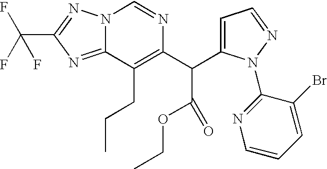

[2-(3-Bromo-pyridin-2-yl)-2H-pyrazol-3-yl]-{5-propyl-6-[N′-(2,2,2-trifluoro-acetyl)hydrazino]-pyrimidin-4-yl}-acetic acid ethyl ester (128) is dissolved in 5 mL of phosphorous oxychloride, and stirred at 100° C. for 2 hours. The excess phosphorous oxychloride is removed. The residue is diluted with 60 mL of EtOAc, and basified with saturated aqueous NaHCO₃. The organic layer is collected, washed with water and brine, dried over Na₂SO₄, and concentrated. The crude is purified through silica gel chromatography (hexanes/EtOAc, from 4:1 to 2:1) to give the product 129.

### Step 4. 7-[2-(3-bromo-pyridin-2-yl)-2H-pyrazol-3-yl]-8-propyl-2-trifluoromethyl-[1,2,4]triazolo[1,5-c]pyrimidine (130)

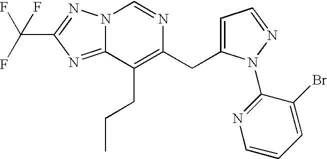

[2-(3-Bromo-pyridin-2-yl)-2H-pyrazol-3-yl]-(8-propyl-2-trifluoromethyl-[1,2,4]triazolo[1,5-c]pyrimidin-7-yl)-acetic ethyl ester (129) (790 mg, 1.47 mmol) is dissolved in 50 mL of 6.0N hydrochloric acid, stirred at 60° C. for 3 hours, and then cooled to 0° C. and basified with saturated aqueous NaHCO₃. The mixture is extracted with EtOAc (40 mL×3). The organic layer is washed with water and brine, dried over Na₂SO₄, and concentrated. The crude is purified through silica gel chromatography (hexanes/EtOAc, from 4:1 to 2:1) to give the product 130.

### Step 5. 2-(5-{[2-(difluoromethyl)-8-propyl[1,2,4]triazolo[1,5-c]pyrimidin-7-yl]methyl}-1H-pyrazol-1-yl)nicotinonitrile (131)

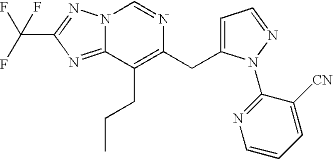

Compound 131 is synthesized via a procedure similar to that illustrated by Example 1D (step 3) using 7-[2-(3-bromo-pyridin-2-yl)-2H-pyrazol-3-yl]-8-propyl-2-trifluoromethyl-[1,2,4]triazolo[1,5-c]pyrimidine (130). ¹H NMR (300 MHz, CDCl₃) δ 9.15 (1H, s), 8.55 (1H, dd, J=1.8 Hz), 8.17 (1H, dd, J=1.5, 7.5 Hz), 7.77 (1H, d, J=1.8 Hz), 7.35 (1H, dd, J=4.8, 7.8 Hz), 6.20 (1H, d, J=1.8 Hz), 4.73 (2H, s), 3.06 (2H, m), 1.75 (2H, m), 1.04 (3H, t, J=7.2 Hz).

### C. Synthesis of Additional [1,2,4]TRIAZOLO[1,5-c]PYRIMIDINES

The compounds shown in Table 2 are synthesized via methods provided in Schemes 7 and 8 and further illustrated by Examples 2A and 2B.

TABLE 2

| | Compound | Name | LC-MS/NMR |
|---|---|---|---|
| 132 | 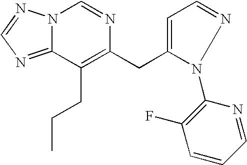 | 7-{[1-(3-Fluoropyridin-2- yl)-1H-pyrazol-5- yl]methyl}-8- propyl[1,2,4]triazolo[1,5- c]pyrimidine | ¹H NMR δ (CDCl₃) 0.99 (t, 3H), 1.60-1.71 (m, 2H), 2.92 (t, 2H), 4.47 (s, 2H), 6.16 (s, 1H), 7.33-7.39 (m, 1H), 7.62 (td, 1H), 7.69 (d, 1H), 8.33 (s, 1H), 8.34 (s, 1H), 9.12 (s, 1H). |
| 133 | 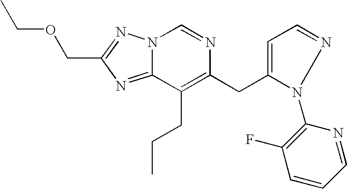 | 2-(Ethoxymethyl)-7-{[1-(3- fluoropyridin-2-yl)-1H- pyrazol-5-yl]methyl}-8- propyl[1,2,4]triazolo[1,5- c]pyrimidine | ¹H NMR δ (CDCl₃) 0.96 (t, 3H), 1.29 (t, 3H), 1.61-1.69 (m, 2H), 2.89 (t, 2H), 3.70 (q, 2H), 4.43 (s, 2H), 4.78 (s, 2H), 6.15 (s, 1H), 7.33-7.38 (m, 1H), 7.61 (t, 1H), 7.69 (s, 1H), 8.31 (d, 1H), 9.06 (s, 1H). |
| 134 | 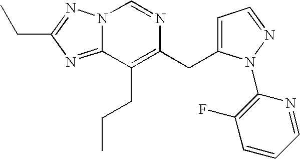 | 2-Ethyl-7-{[1-(3- fluoropyridin-2-yl)-1H- pyrazol-5-yl]methyl}-8- propyl[1,2,4]triazolo[1,5- c]pyrimidine | ¹H NMR δ (CDCl₃) 0.97 (t, 3H), 1.43 (t, 3H), 1.60-1.67 (m, 2H), 2.85-2.98 (m, 4H), 4.42 (s, 2H), 6.15 (s, 1H), 7.37-7.39 (m, 1H), 7.62 (t, 1H), 7.69 (s, 1H), 8.34 (s, 1H), 9.01 (s, 1H). |
| 135 | 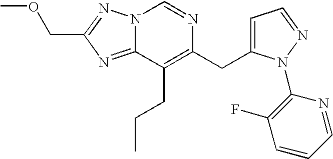 | 7-{[1-(3-Fluoropyridin-2- yl)-1H-pyrazol-5- yl]methyl}-2- (methoxymethyl)-8- propyl[1,2,4]triazolo[1,5- c]pyrimidine | ¹H NMR δ (CDCl₃) 0.97 (t, 3H), 1.63-1.70 (m, 2H), 2.90 (t, 2H), 4.47 (s, 2H), 5.54 (s, 2H), 4.44 (s, 3H), 4.74 (s, 2H), 6.17 (s, 1H), 7.33-7.39 (m, 1H), 7.62 (td, 1H), 7.70 (s, 1H), 8.33 |
| #(m, 1H), 8.34 (s, 1H), 9.01 (s, 1H). | | | |
| 136 | 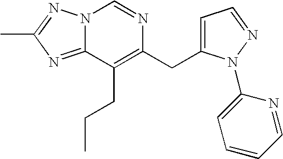 | 2-methyl-8-propyl-7-[(1- pyridin-2-yl-1H-pyrazol-5- yl)methyl][1,2,4]triazolo[1, 5-c]pyrimidine | ¹H NMR δ (CDCl₃) 1.01 (t, 3H), 1.69-1.80 (m, 2H), 2.62 (s, 3H), 3.01 (t, 2H), 4.81 (s, 2H), 6.08 (s, 1H), 7.10-7.20 (m, 1H), 7.62 (s, 1H), 7.79 (t, 1H), 7.90-8.00 (m, 1H), 8.30-8.40 (m, 1H), 9.02 (s, 1H). |
| 137 | 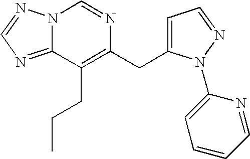 | 8-propyl-7-[(1-pyridin-2- yl-1H-pyrazol-5- yl)methyl][1,2,4]triazolo[1, 5-c]pyrimidine | ¹H NMR δ (CDCl₃) 1.02 (t, 3H), 1.69-1.80 (m, 2H), 3.05 (t, 2H), 4.84 (s, 2H), 6.09 (s, 1H), 7.11-7.20 (m, 1H), 7.62 (d, 1H), 7.79 (t, 1H), 7.95 (d, 1H), 8.32 (d, 1H), 8.53 (s, 1H), 9.02 (s, 1H). |
| 138 | 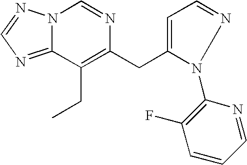 | 8-Ethyl-7-{[1-(3- fluoropyridin-2-yl)-1H- pyrazol-5- yl]methyl}[1,2,4]triazolo[1, 5-c]pyrimidine | ¹H NMR δ (CDCl₃) 1.25 (t, 3H), 2.98 (q, 2H), 4.45 (s, 2H), 6.15 (s, 1H), 7.33-7.38 (m, 1H), 7.62 (t, 1H), 7.69 (s, 1H), 8.35 (s, 2H), 9.12 (s, 1H). |
| 139 | 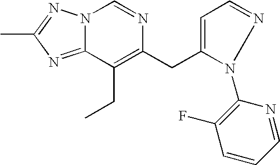 | 8-Ethyl-7-{[1-(3- fluoropyridin-2-yl)-1H- pyrazol-5-yl]methyl}-2- methyl[1,2,4]triazolo[1,5- c]pyrimidine | ¹H NMR δ (CDCl₃) 1.23 (t, 3H), 2.59 (s, 3H), 2.93 (q, 2H), 4.42 (s, 2H), 6.13 (s, 1H), 7.24-7.38 (m, 1H), 7.61 (t, 1H), 7.68 (s, 1H), 8.32 (s, 1H), 8.98 (s, 1H). |
| 140 | 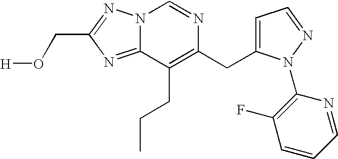 | (7-{[1-(3-Fluoropyridin-2- yl)-1H-pyrazol-5- yl]methyl}-8- propyl[1,2,4]triazolo[1,5- c]pyrimidin-2-yl)methanol | ¹H NMR δ (CDCl₃) 0.97 (t, 3H), 1.61-1.68 (m, 2H), 2.89 (t, 2H), 3.45 (s, 1H), 4.44 (s, 2H), 4.96 (s, 2H), 6.15 (s, 1H), 7.53-7.39 (m, 1H), 7.62 (t, 1H), 7.69 (d, 1H), 8.33 (d, 1H), 9.06 (s, 1H). |
| 141 | 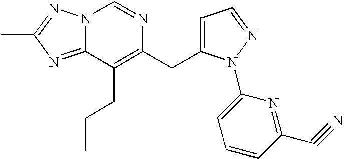 | 6-{5-[(2-methyl-8- propyl[1,2,4]triazolo[1,5- c]pyrimidin-7-yl)methyl]- 1H-pyrazol-1-yl}pyridine- 2-carbonitrile | ¹H NMR δ (CDCl₃) 1.05 (t, 3H), 1.73-1.83 (m, 2H), 2.63 (s, 3H), 3.04 (t, 2H), 4.83 (s, 2H), 6.07 (d, 1H), 7.54 (d, 1H), 7.66 (d, 1H), 7.94 (t, 1H), 8.30 (d, 1H), 9.02 (s, 1H) |
| 142 | 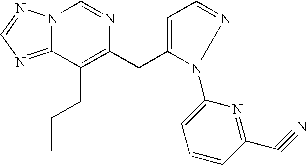 | 6-{5-[(8- propyl[1,2,4]triazolo[1,5- c]pyrimidin-7-yl)methyl]- 1H-pyrazol-1-yl}pyridine- 2-carbonitrile | ¹H NMR δ (CDCl₃) 1.06 (t, 3H), 1.73-1.85 (m, 2H), 3.01 (t, 2H), 4.86 (s, 2H), 6.09 (d, 1H), 7.54 (d, 1H), 7.67 (s, 1H), 7.95 (t, 1H), 8.31 (d, 1H), 8.39 (s, 1H), 9.15 (s, 1H). |
| 143 | 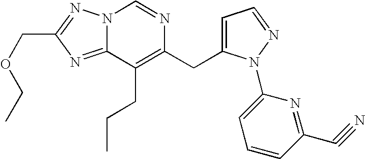 | 6-(5-{[2-(ethoxymethyl)-8- propyl[1,2,4]triazolo[1,5- c]pyrimidin-7-yl]methyl}- 1H-pyrazol-1-yl)pyridine- 2-carbonitrile | ¹H NMR δ (CDCl₃) 1.04 (t, 3H), 1.31 (t, 3H), 1.73-1.85 (m, 2H), 3.07 (t, 2H), 3.72 (q, 2H), 4.81 (s, 2H), 4.82 (s, 2H), 6.06 (s, 1H), 7.53 (d, 1H), 7.65 (s, 1H), 7.93 (t, 1H), 8.28 (d, 1H), 9.09 |
| # (s, 1H). | | | |
| 144 | 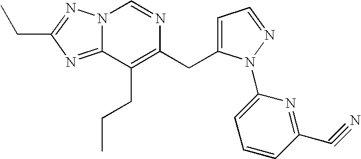 | 6-{5-[(2-ethyl-8- propyl[1,2,4]triazolo[1,5- c]pyrimidin-7-yl)methyl]- 1H-pyrazol-1-yl}pyridine- 2-carbonitrile | ¹H NMR δ (CDCl₃) 1.04 (t, 3H), 1.42 (t, 3H), 1.73-1.85 (m, 2H), 2.92-3.08 (m, 4H), 4.82 (s, 2H), 6.05 (d, 1H), 7.54 (d, 1H), 7.64 (s, 1H), 7.93 (t, 1H), 8.28 (d, 1H), 9.04 (s, 1H). |
| 145 | 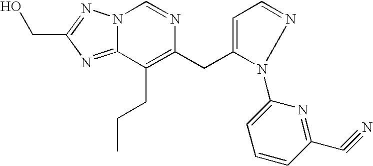 | 6-(5-{[2-(hydroxymethyl)- 8-propyl[1,2,4]triazolo[1,5- c]pyrimidin-7-yl]methyl}- 1H-pyrazol-1-yl)pyridine- 2-carbonitrile | ¹H NMR δ (CDCl₃) 1.05 (t, 3H), 1.73-1.85 (m, 2H), 3.07 (t, 2H), 4.84 (s, 2H), 4.98 (s, 2H), 6.09 (s, 1H), 7.53 (d, 1H), 7.66 (d, 1H), 7.93 (t, 1H), 8.30 (d, 1H), 9.07 (s, 1H). |
| 146 | 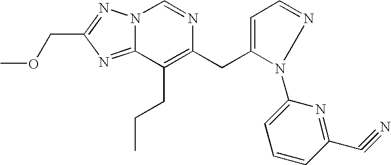 | 6-(5-{[2-(methoxymethyl)- 8-propyl[1,2,4]triazolo[1,5- c]pyrimidin-7-yl]methyl}- 1H-pyrazol-1-yl)pyridine- 2-carbonitrile | ¹H NMR δ (CDCl₃) 1.04 (t, 3H), 1.73-1.83 (m, 2H), 3.07 (t, 2H), 3.56 (s, 3H), 4.77 (s, 2H), 4.84 (s, 2H), 6.07 (s, 1H), 7.53 (d, 1H), 7.66 (s, 1H), 7.93 (t, 1H), 8.29 (d, 1H), 9.08 (s, 1H). |
| 147 | 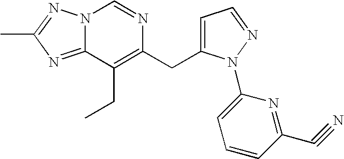 | 6-{5-[(8-ethyl-2- methyl[1,2,4]triazolo[1,5- c]pyrimidin-7-yl)methyl]- 1H-pyrazol-1-yl}pyridine- 2-carbonitrile | ¹H NMR δ (CDCl₃) 1.34 (t, 3H), 2.63 (s, 3H), 3.09 (q, 2H), 4.82 (s, 2H), 6.04 (d, 1H), 7.08 (t, 1H), 7.54 (d, 1H), 7.64 (d, 1H), 7.93 (t, 1H), 8.29 (d, 1H), 9.02 (s, 1H). |
| 148 | 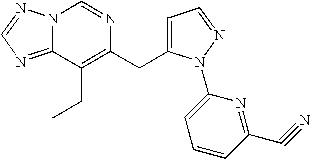 | 6-{5-[(8- ethyl[1,2,4]triazolo[1,5- c]pyrimidin-7-yl)methyl]- 1H-pyrazol-1-yl}pyridine- 2-carbonitrile | ¹H NMR δ (CDCl₃) 1.35 (t, 3H), 3.14 (q, 2H), 4.85 (s, 2H), 6.07 (d, 1H), 7.26 (d, 1H), 7.53 (d, 1H), 7.64 (d, 1H), 7.93 (t, 1H), 8.31 (d, 1H), 8.38 (s, 1H), 9.14 (s, 1H). |
| 149 | 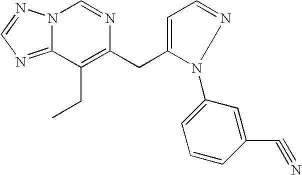 | 3-{5-[(8- ethyl[1,2,4]triazolo[1,5- c]pyrimidin-7-yl)methyl]- 1H-pyrazol-1- yl}benzonitrile | ¹H NMR δ (CDCl₃) 1.36 (t, 3H), 3.15 (q, 2H), 4.32 (s, 2H), 6.40 (d, 1H), 7.52-7.54 (m, 2H), 7.85-7.90 (m, 1H), 7.97 (m, 1H), 8.37 (s, 1H), 9.25 (s, 1H). |
| 150 | 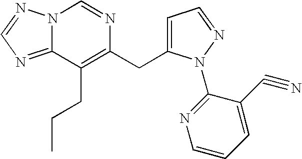 | 2-{5-[(8- propyl[1,2,4]triazolo[1,5- c]pyrimidin-7-yl)methyl]- 1H-pyrazol-1- yl}nicotinonitrile | ¹H NMR (300 MHz, CDCl₃) δ 9.12 (1H, s), 8.58 (1H, dd, J = 2.1, 5.1 Hz), 8.36 (1H, s), 8.17 (1H, dd, J = 2.1, 7.8 Hz), 7.76 (1H, d, J = 1.5 Hz), 7.35 (1H. dd, J = 5.1, 7.5 Hz), 6.18 (1H, d, J = 1.2 |
| #Hz), 4.69 (2H, s), 3.02 (2H, t, J = 8.1 Hz), 1.77 (2H, m), 1.04 (3H, t, J = 7.2 Hz) | | | |
| 151 | 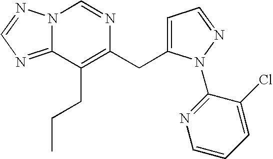 | 7-{[1-(3-chloropyridin-2- yl)-1H-pyrazol-5- yl]methyl}-8- propyl[1,2,4]triazolo[1,5- c]pyrimidine | ¹H NMR (300 MHz, CDCl₃) δ 9.11 (1H, s), 8.46 (1H, dd, J = 1.8, 7.8 Hz), 8.34 (1H, s), 7.89 (1H, dd, J = 1.5, 8.1 Hz), 7.69 (1H, d, J = 1.5 Hz), 7.35 (1H. dd, J = 4.8, 8.1 Hz), 6.21 (1H, d, J = |
| #1.5 Hz), 4.31 (2H, s), 2.85 (2H, m), 1.63 (2H, m), 0.97 (3H, t, J = 7.2 Hz) | | | |
| 152 | 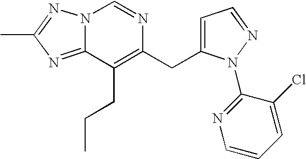 | 7-{[1-(3-chloropyridin-2- yl)-1H-pyrazol-5- yl]methyl}-2-methyl-8- propyl[1,2,4]triazolo[1,5- c]pyrimidine | ¹H NMR (300 MHz, CDCl₃) δ 8.99 (1H, s), 8.46 (1H, dd, J = 1.5, 4.8 Hz), 7.88 (1H, dd, J = 1.5, 8.1 Hz), 7.68 (1H, d, J = 1.8 Hz), 7.34 (1H, dd, J = 4.8, 8.1 Hz), 6.19 (1H, d, J = 1.8 |
| #Hz), 4.28 (2H, s), 2.76 (2H, t, J = 7.8 Hz), 1.63 (2H, m), 0.96 (3H, t, J = 7.2 Hz) | | | |
| 153 | 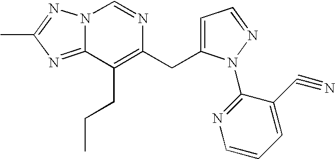 | 2-{5-[(2-methyl-8- propyl[1,2,4]triazolo[1,5- c]pyrimidin-7-yl)methyl]- 1H-pyrazol-1- yl}nicotinonitrile | ¹H NMR (400 MHz, CDCl₃) δ 8.99 (1H, s), 8.57 (1H, dd, J = 2.0, 4.8 Hz), 8.15 (1H, dd, J = 12.0, 7.6 Hz), 7.75 (1H, d, J = 1.6 Hz), 7.33 (1H, dd, J = 4.8, 7.6 Hz), 6.17 (1H, d, J = 2.0 |
| #Hz), 4.66 (2H, s), 2.97 (2H, t, m), 1.74 (2H, m), 1.02 (3H, t, J = 7.6 Hz) | | | |
| 154 | 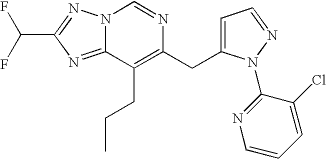 | 7-{[1-(3-chloropyridin-2- yl)-1H-pyrazol-5- yl]methyl}-2- (difluoromethyl)-8- propyl[1,2,4]triazolo[1,5- c]pyrimidine | LC-MS (M + 1): 404.82. |
| 155 | 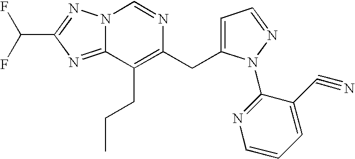 | 2-(5-{[2-(difluoromethyl)- 8-propyl[1,2,4]triazolo[1,5- c]pyrimidin-7-yl]methyl}- 1H-pyrazol-1- yl)nicotinonitrile | ¹H NMR (300 MHz, CDCl₃) δ 9.12 (1H, s), 8.56 (1H, d, J = 2.1, 5.1 Hz), 8.17 (1H, dd, J = 1.8, 7.8 Hz), 7.76 (1H, d, J = 2.1 Hz), 7.35 (1H, dd, J = 4.5, 7.8 Hz), 6.88 (1H, t, J = 53.4 |
| #Hz), 6.20 (1H, dd, J = 0.9, 1.5 Hz), 4.72 (2H, s), 3.05 (2H, t, m), 1.75 (2H, m), 1.04 (3H, t, J = 7.5 Hz) | | | |
| 156 | 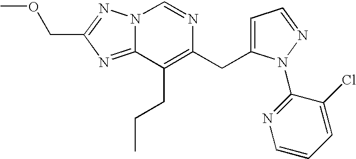 | 7-{[1-(3-chloropyridin-2- yl)-1H-pyrazol-5- yl]methyl}-2- (methoxymethyl)-8- propyl[1,2,4]triazolo[1,5- c]pyrimidine | LC-MS (M + 1): 398.86. |
| 157 | 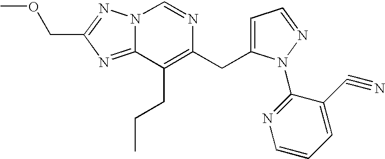 | 2-(5-{[2-(methoxymethyl)- 8-propyl[1,2,4]triazolo[1,5- c]pyrimidin-7-yl]methyl}- 1H-pyrazol-1- yl)nicotinonitrile | ¹H NMR (300 MHz, CDCl₃) δ 9.05 (1H, s), 8.56 (1H, dd, J = 1.8, 4.8 Hz), 8.15 (1H, dd, J = 1.8, 8.1 Hz), 7.75 (1H, d, J = 1.8 Hz), 7.33 (1H, dd, J = 4.5, 7.5 Hz), 6.18 (1H, d, J = |
| # 1.5 Hz), 4.75 (2H, s), 4.67 (2H, s), 3.55 (3H, s), 3.00 (2H, m), 1.74 (2H, m), 1.02 (3H, t, J = 7.2 Hz) | | | |
| 158 | 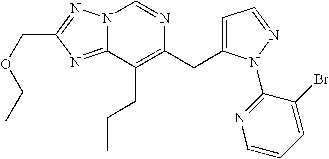 | 7-{[1-(3-bromopyridin-2- yl)-1H-pyrazol-5- yl]methyl}-2- (ethoxymethyl)-8- propyl[1,2,4]triazolo[1,5- c]pyrimidine | LC-MS (M + 1): 457.54. |
| 159 | 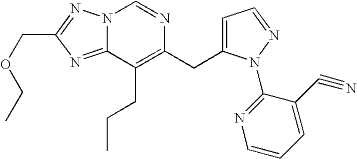 | 2-(5-{[2-(ethoxymethyl)-8- propyl[1,2,4]triazolo[1,5- c]pyrimidin-7-yl]methyl}- 1H-pyrazol-1- yl)nicotinonitrile | ¹H NMR (400 MHz, CDCl₃) δ 9.05 (1H, s), 8.56 (1H, dd, J = 1.8, 4.8 Hz), 8.15 (1H, dd, J = 1.5, 7.8 Hz), 7.75 (1H, d, J = 1.8 Hz), 7.33 (1H, dd, J = 4.8, 7.8 Hz), 6.17 (1H, d, |
| #J = 1.5 Hz), 4.78 (2H, s), 4.67 (2H, s), 3.72 (2H, q, J = 7.2 Hz), 3.00 (2H, m), 1.72 (2H, m), 1.30 (3H, t, J = 6.9Hz), 1.02 (3H, t, J = 7.5 Hz). | | | |
| 160 | 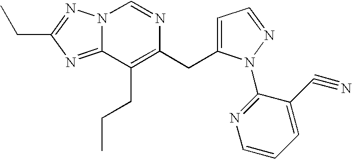 | 2-{5-[(2-ethyl-8- propyl[1,2,4]triazolo[1,5- c]pyrimidin-7-yl)methyl]- 1H-pyrazol-1- yl}nicotinonitrile | ¹H NMR (300 MHz, CDCl₃) δ 9.01 (1H, s), 8.58 (1H, dd, J = 1.8, 4.8 Hz), 8.15 (1H, dd, J = 1.8, 7.8 Hz), 7.74 (1H, d, J = 1.8 Hz), 7.34 (1H, dd, J = 4.8, 7.8 Hz), 6.16 (1H, d, J = 1.5 Hz), |
| #4.75 (2H, s), 4.65 (2H, s), 2.96 (4H, m, overlapped), 1.73 (2H, m), 1.42 (3H, t, J = 7.2 Hz), 1.02 (3H, t, J = 7.2 Hz) | | | |
| 161 | 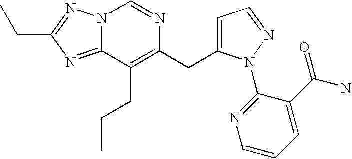 | 2-{5-[(2-ethyl-8- propyl[1,2,4]triazolo[1,5- c]pyrimidin-7-yl)methyl]- 1H-pyrazol-1- yl}nicotinamide | LC-MS (M + 1): 391.44. |
| 162 | 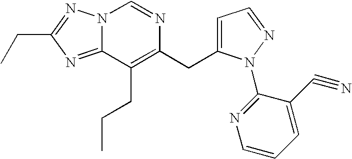 | 2-(5-{[2-(hydroxymethyl)- 8-propyl[1,2,4]triazolo[1,5- c]pyrimidin-7-yl]methyl}- 1H-pyrazol-1- yl)nicotinonitrile | ¹H NMR (300 MHz, CDCl₃) δ 9.05 (1H, s), 8.57 (1H, dd, J = 1.8, 4.8 Hz), 8.16 (1H, dd, J = 1.8, 7.5 Hz), 7.76 (1H, d, J = 1.5 Hz), 7.34 (1H, dd, J = 4.8, 7.8 Hz), 6.18 (1H, d, J = |
| #1.5 Hz), 4.98 (2H, d, J = 6.3 Hz), 4.68 (2H, s), 3.00 (2H, m), 1.73 (2H, m), 1.03 (3H, t, J = 7.2 Hz) | | | |
| 163 | 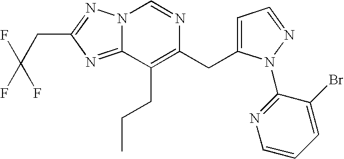 | 7-{[1-(3-bromopyridin-2- yl)-1H-pyrazol-5- yl]methyl}-8-propyl-2- (2,2,2- trifluoroethyl)[1,2,4]triazolo [1,5-c]pyrimidine | LC-MS (M + 1): 481.29. |
| 164 | 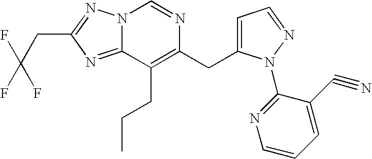 | 2-(5-{[8-propyl-2-(2,2,2- trifluoroethyl)[1,2,4]triazolo [1,5-c]pyrimidin-7- yl)methyl}-1H-pyrazo1-1- yl)nicotinonitrile | ¹H NMR (300 MHz, CDCl₃) δ 9.07 (1H, s), 8.58 (1H, dd, J = 1.8, 4.5 Hz), 8.16 (1H, dd, J = 2.1, 8.1 Hz), 7.75 (1H, d, J = 1.8 Hz), 7.35 (1H, dd, J = 4.8, 7.8 Hz), 6.18 (1H, d, |
| #J = 1.8 Hz), 4.68 (2H, s), 3.78 (2H, t, J = 10.2 Hz), 3.00 (2H, m), 1.74 (2H, m), 1.02 (3H, t, J = 7.2 Hz) | | | |
| 165 | 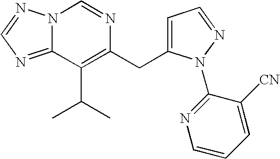 | 2-{5-[(8- isopropyl[1,2,4]triazolo[1,5- c]pyrimidin-7-yl)methyl]- 1H-pyrazol-1- yl}nicotinonitrile | ¹H NMR (300 MHz, CDCl₃) δ 9.13 (1H, s), 8.62-8.60 (1H, m), 8.37 (1H, s), 8.20-8.17 (1H, m), 7.73 (1H, d), 7.39-7.34 (1H, m), 6.06 (1H, d), 4.72 (2H, s), 3.46-3.37 (1H, m), 1.52 (6H, d). |
| 166 | 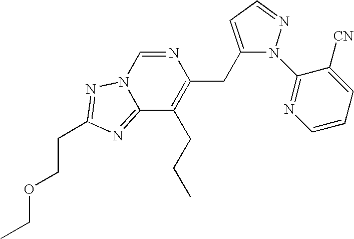 | 2-{5-[2-(2-ethyoxy-ethy)- 8-propyl- [1,2,4]triazolo[1,5-c]- pyrimidin-7-ylmethyl}- pyrazol-1-yl}- nicotinointrile | ¹H NMR δ (CDCl₃): 9.01 (s, 1H), 8.57 (dd, 1H), 8.16 (dd, 1H), 7.74 (d, 1H), 7.34 (dd, 1H), 6.16 (d, 1H), 4.65 (s, 2H), 3.92 (t, 2H), 3.56 (q, 2H), 3.21 (t, 2H), 2.94-3.00 (m, 2H), 1.67-1.79 |
| #(m, 2H), 1.17 (t, 3H), 1.01 (t, 3H). | | | |
| 167 | 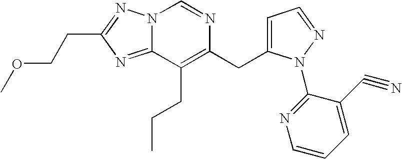 | 2-(5-{[2-(2- methoxyethyl)-8- propyl[1,2,4]triazolo[1,5- c]pyrimidin-7-yl]methyl}- 1H-pyrazol-1- yl)nicotinonitrile | ¹H NMR (300 MHz, CDCl₃) δ 9.02 (1H, s), 8.57 (1H, dd, J = 2.1, 5.1 Hz), 8.16 (1H, dd, J = 2.1, 7.8 Hz), 7.75 (1H, d, J = 1.5 Hz), 7.34 (1H. dd, J = 5.1, 7.8 Hz), 6.16 (1H, d, |
| #J = 1.5 Hz), 4.66 (2H, s), 3.89 (2H, t, J = 6.6 Hz), 3.39 (3H, s), 3.21 (2H, t, J = 6.6 Hz), 2.97 (2H, t, J = 8.1 Hz), 1.73 (2H, m), 1.02 (3H, t, J = 7.5 Hz) | | | |
| 168 | 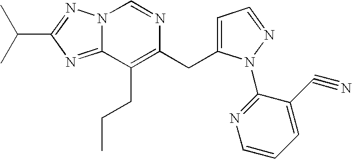 | 2-{5-[(2-isopropyl-8- propyl[1,2,4]triazolo[1,5- c]pyrimidin-7-yl)methyl]- 1H-pyrazol-1- yl}nicotinonitrile | ¹H NMR (300 MHz, CDCl₃) δ 9.02 (1H, s), 8.59 (1H, dd, J = 1.8, 4.8 Hz), 8.16 (1H, dd, J = 1.5, 7.5 Hz), 7.74 (1H, d, J = 1.5 Hz), 7.34 (1H, dd, J = 5.1, 7.8 Hz), 6.16 (1H, d, J = 1.8 |
| #Hz), 4.65 (2H, s), 3.26 (1H, m), 2.98 (2H, m), 1.73 (2H, m), 1.43 (6H, d, J = 6.9 Hz), 1.02 (3H, t, J = 7.2 Hz). | | | |
| 169 | 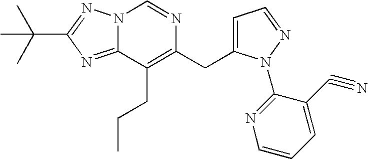 | 2-{5-[(2-tert-butyl-8- propyl[1,2,4]triazolo[1,5- c]pyrimidin-7-yl)methyl]- 1H-pyrazol-1- yl}nicotinonitrile | ¹H NMR (300 MHz, CDCl₃) δ 9.03 (1H, s), 8.60 (1H, dd, J = 1.8, 4.8 Hz), 8.16 (1H, dd, J = 1.8, 7.8 Hz), 7.73 (1H, d, J = 1.8 Hz), 7.34 (1H, dd, J = 4.8, 7.5 Hz), 6.14 (1H, d, J = 1.5 |
| #Hz), 4.64 (2H, s), 2.98 (2H m), 1.73 (2H, m), 1.46 (9H, s), 1.00 (3H, t, J = 7.2 Hz). | | | |
| 170 | 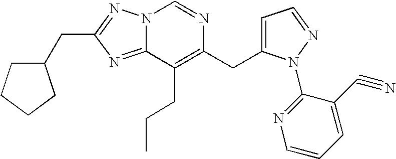 | 2-(5-{[2- (cyclopentylmethyl)-8- propyl[1,2,4]triazolo[1,5- c]pyrimidin-7-yl]methyl}- 1H-pyrazol-1- yl)nicotinonitrile | ¹H NMR (300 MHz, CDCl₃) δ 9.01 (1H, s), 8.59 (1H, dd, J = 1.5, 4.5 Hz), 8.16 (1H, dd, J = 1.5, 7.5 Hz), 7.74 (1H, d, J = 1.5 Hz), 7.34 (1H, dd, J = 4.8, 7.8 Hz), 6.17 |
| # (1H, d, J = 1.5 Hz), 4.65 (2H, s), 2.98 (2H, m), 2.91 (2H, d, J = 7.5 Hz), 2.41 (1H, m), 1.50˜1.85 (8H, m), 1.30 (2H, m), 1.02 (3H, t, J = 7.2 Hz). | | | |
| 171 | 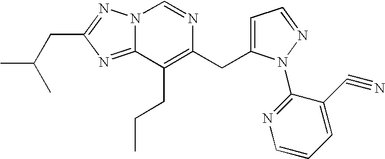 | 2-{5-[(2-isobutyl-8- propyl[1,2,4]triazolo[1,5 c]pyrimidin-7-yl)methyl]- 1H-pyrazol-1- yl}nicotinonitrile | ¹H NMR (400 MHz, CDCl₃) δ 9.01 (1H, s), 8.58 (1H, dd, J = 2.0, 4.8 Hz), 8.16 (1H, dd, J = 2.0, 8.0 Hz), 7.74 (1H, d, J = 1.6 Hz), 7.34 (1H, dd, J = 4.8, 7.6 Hz), 6.17 (1H, d, J =2.0 Hz), |
| #4.65 (2H, s), 2.98 (2H, m), 2.79 (2H, d, J = 6.8 Hz), 2.24 (1H, m), 1.73 (2H, m), 0.99˜1.04 (9H, m) | | | |

### D. 7-{[1-(3-CYANOPYRIDIN-2-YL)-1H-PYRAZOL-5-YL]METHYL}-8-PROPYL[1,2,4]TRIAZOLO [1,5-c]PYRIMIDINE-2-CARBOXAMIDE (176)

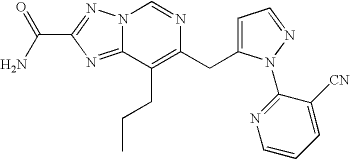

### Step 1. {6-[2-(3-bromo-pyridin-2-yl)-2H-pyrazol-3-ylmethyl]-5-propyl-3,4-dihydro-pyrimidin-4-yl}-hydrazine (172)

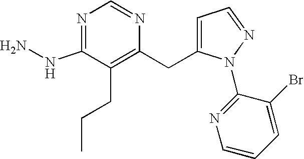

A solution of [2-(3-bromo-pyridin-2-yl)-2H-pyrazol-3-yl]-(6-hydrazino-5-propyl-pyrimidin-4-yl)-acetic acid ethyl ester (127) (1.28 g, 12.78 mmol) in 30 mL of 6.0N hydrochloric acid is stirred at 60° C. for 3 hours, and then cooled to 0° C. and basified with a saturated aqueous NaHCO₃. The mixture is extracted with DCM (30 mL×3), dried over Na₂SO₄, and concentrated to give crude 172.

### Step 2. (N′-{6-[2-(3-bromo-pyridin-2-yl)-2H-pyrazol-3-ylmethyl]-5-propyl-3,4-dihydro-pyrimidin-4-yl}-hydrazino)-oxo-acetic acid ethyl ester (173)

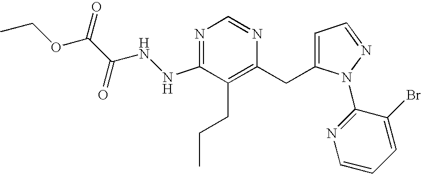

To a solution of {6-[2-(3-bromo-pyridin-2-yl)-2H-pyrazol-3-ylmethyl]-5-propyl-3,4-dihydro-pyrimidin-4-yl}-hydrazine (172) (500 mg, 1.28 mmol) in 30 mL of anhydrous DCM containing 2.0 equivalent of trimethylamine at 0° C. is added ethyl chlorooxoacetate (281 mg, 1.92 mmol, 1.5 eq.) dropwise. The resulting mixture is stirred at room temperature for 2 hours. 10 mL of water is added to quench the reaction, and the mixture is washed with saturated aqueous NaHCO₃, dried over Na₂SO₄ and concentrated to give crude 173.

### Step 3. 7-[2-(3-bromo-pyridin-2-yl)-2H-pyrazol-3-ylmethyl]-8-propyl-[1,2,4]triazolo[1,5-c]pyrimidine-2-carboxylic acid ethyl ester (174)

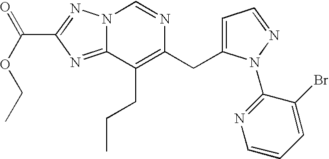

A solution of crude 173 in 5 mL of acetic acid is heated at 100° C. overnight. The acetic acid is removed. The residue is diluted with 50 mL of DCM, washed with saturated aqueous NaHCO₃, dried over Na₂SO₄ and concentrated. The crude is purified through silica gel chromatography (hexanes/EtOAc, 2:1) to give compound 174.

### Step 4. 7-[2-(3-bromo-pyridin-2-yl)-2H-pyrazol-3-ylmethyl]-8-propyl-[1,2,4]triazolo[1,5-c]pyrimidine-2-carboxamide (175)

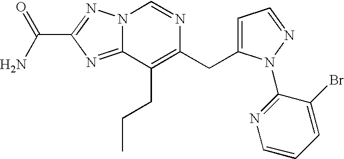

7-[2-(3-bromo-pyridin-2-yl)-2H-pyrazol-3-ylmethyl]-8-propyl-[1,2,4]triazolo[1,5-c]pyrimidine-2-carboxylic acid ethyl ester (174) (100 mg, 0.212 mmol) is dissolved in 10 mL of 7.0N ammonium MeOH solution. The solution is stirred at 80° C. overnight. The solvent is removed, and the residue is diluted with 30 mL of DCM, washed with water and brine, dried over Na₂SO₄ and concentrated. The crude is purified through silica gel chromatography (hexanes/EtOAc, 1:1, plus 5% MeOH) to give compound 175.

### Step 5. 7-{[1-(3-cyanopyridin-2-yl)-1H-pyrazol-5-yl]methyl}-8-propyl[1,2,4]triazolo[1,5-c]pyrimidine-2-carboxamide (176)

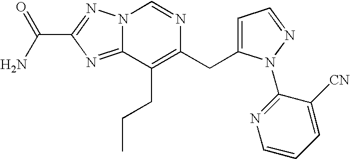

The compound 176 is synthesized via a procedure similar to that illustrated by Example 1D (step 3) using compound 175 as a starting material. ¹H NMR (300 MHz, CDCl₃) δ 9.13 (1H, s), 8.56 (1H, dd, J=1.8, 4.8 Hz), 8.17 (1H, dd, J=1.8, 7.8 Hz), 7.77 (1H, d, J=1.8 Hz), 7.35 (1H, dd, J=4.8, 7.8 Hz), 6.20 (1H, s), 5.79 (2H, s, br), 4.75 (2H, s), 4.72 (2H, s), 3.04 (2H, m), 1.75 (2H, m), 1.04 (3H, t, J=7.2 Hz).

Compounds 177 and 178 are synthesized via a similar procedure.

### 7-{[1-(3-BROMOPYRIDIN-2-YL)-1H-PYRAZOL-5-YL]METHYL}-N-METHYL-8-PROPYL[1,2,4]TRIAZOLO[1,5-C]PYRIMIDINE-2-CARBOXAMIDE (177)

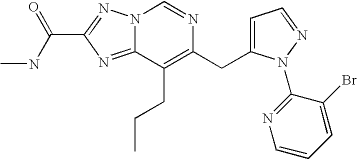

¹H NMR (300 MHz, CDCl₃) δ 9.12 (1H, s), 8.49 (1H, dd, J=1.8, 4.8 Hz), 8.05 (1H, dd, J=1.5, 7.8 Hz), 7.69 (1H, d, J=1.8 Hz), 7.40 (1H, s, br), 7.26 (1H, dd, J=4.8, 7.8 Hz), 6.23 (1H, d, J=1.5 Hz), 4.31 (2H, s), 3.09 (3H, d, J=5.1 Hz), 2.82 (2H, t, J=7.8 Hz), 1.63 (2H, m), 0.97 (3H, t, J=7.2 Hz).

### 7-{[1-(3-CYANOPYRIDIN-2-YL)-1H-PYRAZOL-5-YL]METHYL}-N-METHYL-8-PROPYL[1,2,4]TRIAZOLO[1,5-C]PYRIMIDINE-2-CARBOXAMIDE (178)

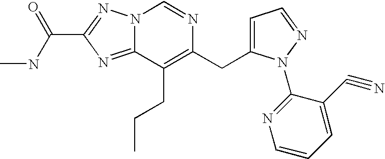

¹H NMR (400 MHz, CDCl₃) δ 9.12 (1H, s), 8.55 (1H, dd, J=1.6, 4.8 Hz), 8.17 (1H, dd, J=1.5, 7.6 Hz), 7.75 (1H, d, J=1.6 Hz), 7.41 (1H, s, br), 7.34 (1H, dd, J=4.8, 8.0 Hz), 6.19 (1H, d, J=1.6 Hz), 4.70 (2H, s), 3.10 (3H, d, J=5.2 Hz), 3.01 (2H, t, m), 1.74 (2H, m), 1.03 (3H, t, J=7.6 Hz).

### E. 2-(5-{[2-(1-HYDROXY-1-METHYLETHYL)-8-PROPYL[1,2,4]TRIAZOLO[1,5-c]PYRIMIDIN-7-YL]METHYL}-1H-PYRAZOL-1-YL)NICOTINONITRILE (180)

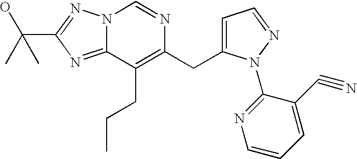

### Step 1. 2-(7-{[1-(3-bromopyridin-2-yl)-1H-pyrazol-5-yl]methyl}-8-propyl[1,2,4]triazolo[1,5-c]pyrimidin-2-yl)propan-2-ol (179)

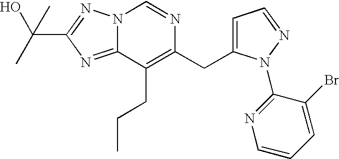

To a solution of 7-[2-(3-bromo-pyridin-2-yl)-2H-pyrazol-3-ylmethyl]-8-propyl-[1,2,4]triazolo[1,5-c]pyrimidine-2-carboxylic acid ethyl ester (174) (138 mg, 0.293 mmol) in 10 mL of anhydrous THF at 0° C. is added methyl magnesium bromide (1.0N, 0.62 mL, 0.62 mmol, 2.1 eq.) dropwise. The resulting mixture is stirred at room temperature for 2 hours. 5 mL of aqueous ammonium chloride solution is added and THF is removed. The residue is extracted with DCM (10 mL×3), washed with brine, and dried over Na₂SO₄. Concentration and purification via preparative silica gel TLC gives 179.

### Step 2. 2-(5-{[2-(1-hydroxy-1-methylethyl)-8-propyl[1,2,4]triazolo[1,5-c]pyrimidin-7-yl]methyl}-1H-pyrazol-1-yl)nicotinonitrile (180)

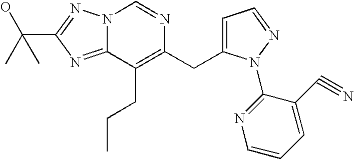

Compound 180 is synthesized via a procedure similar to that illustrated by Example 1D (step 3) using compound 179 as a starting material. ¹H NMR (400 MHz, CDCl₃) δ 9.03 (1H, s), 8.58 (1H, dd, J=1.8, 5.1 Hz), 8.16 (1H, dd, J=1.8, 7.5 Hz), 7.74 (1H, d, J=1.5 Hz), 7.34 (1H, dd, J=4.8, 7.5 Hz), 6.16 (1H, d, J=1.5 Hz), 4.66 (2H, s), 2.97 (2H, m), 1.75 (2H, m), 1.70 (6H, s), 1.01 (3H, t, J=7.5 Hz).

### F. 2-(FLUOROMETHYL)-7-{[1-(3-FLUOROPYRIDIN-2-yl)-1H-PYRAZOL-5-YL]METHYL}-8-PROPYL[1,2,4]TRIAZOLO[1,5-c]PYRIMIDINE (181)

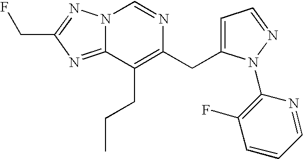

To a solution of (7-{[1-(3-fluoropyridin-2-yl)-1H-pyrazol-5-yl]methyl}-8-propyl[1,2,4]triazolo[1,5-c]pyrimidin-2-yl)methanol (140) (15 mg, 0.04 mmol) in DCM (5 mL) at 0° C. is added bis(2-methoxyethyl)aminosulfur trifluoride (0.2 mL, 50% in THF) under N₂. The mixture is heated at room temperature for 30 minutes and is poured into saturated NaHCO₃ (10 mL). After CO₂ evolution ceases, the mixture is extracted into DCM, dried (MgSO₄), filtered, and the solvent is evaporated in vacuo. PTLC separation with 5% MeOH/DCM gives the pure title product (181). ¹H NMR δ (CDCl₃) 0.98 (t, 3H), 1.63-1.71 (m, 2H), 2.92 (t, 2H), 4.46 (s, 2H), 5.63 (d, 2H), 6.17 (s, 1H), 7.33-7.39 (m, 1H), 7.63 (t, 1H), 7.70 (s, 1H), 8.34 (s, 1H), 9.09 (s, 1H).

### G. 2-(Difluoromethyl)-7-{[1-(3-fluoropyridin-2-yl)-1H-pyrazol-5-yl]methyl}-8-propyl[1,2,4]triazolo[1,5-C]pyrimidine (183)

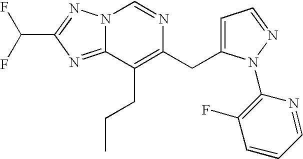

### Step 1. 7-[2-(3-fluoro-pyridin-2-yl)-2H-pyrazol-3-ylmethyl]-8-propyl-[1.2.4]triazolo[1,5-c]pyrimidine-2-carbaldehyde (182)

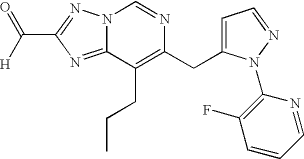

To a solution of (7-{[1-(3-fluoropyridin-2-yl)-1H-pyrazol-5-yl]methyl}-8-propyl[1,2,4]triazolo[1,5-c]pyrimidin-2-yl)methanol (140) (0.162 g) in DCM (15 mL) is added Dess-Martin reagent (0.188 g). The mixture is stirred at room temperature for two hours. Water (10 mL) and DCM (20 mL) are added to the residue. The layers are separated and the organic layer is washed with 1N NaOH (2×10 mL) and brine (15 mL). Upon drying with Na₂SO₄ and filtration, the solvent is evaporated in vacuo. PTLC purification with 5% MeOH/DCM gives the pure title compound 182.

### Step 2. 2-(difluoromethyl)-7-{[1-(3-fluoropyridin-2-yl)-1H-pyrazol-5-yl]methyl}-8-propyl[1,2,4]triazolo[1,5-c]pyrimidine (183)

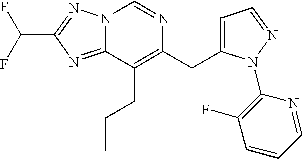

To a solution of 7-[2-(3-fluoro-pyridin-2-yl)-2H-pyrazol-3-ylmethyl]-8-propyl-[1.2.4]triazolo[1,5-c]pyrimidine-2-carbaldehyde (182) (50 mg) in DCM (15 mL) at 0° C. is added bis(2-methoxyethyl)aminosulfur trifluoride (0.5 mL, 50% in THF) under N₂. The mixture is heated at 60° C. for two hours and is poured into saturated NaHCO₃ (10 mL). After CO₂ evolution ceases, the mixture is extracted into DCM, dried (MgSO₄), and filtered. The solvent is evaporated in vacuo. PTLC purification with 5% MeOH/DCM gives the pure title product 183. ¹H NMR δ (CDCl₃) 0.99 (t, 3H), 1.62-1.72 (m, 2H), 2.94 (t, 2H), 4.48 (s, 2H), 6.17 (d, 1H), 6.88 (t, 1H), 7.35-7.38 (m, 1H), 7.63 (t, 1H), 7.70 (d, 2H), 8.33 (d, 1H), 9.13 (s, 1H).

### H. 7-{[1-(3-Fluoropyridin-2-yl)-1H-pyrazol-5-yl]methyl}-8-propyl-2-(pyrrolidin-1-ylmethyl)[1,2,4]triazolo[1,5-c]pyrimidine (185)

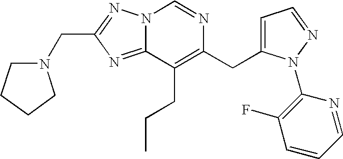

### Step 1. 2-(chloromethyl)-7-{[1-(3-fluoropyridin-2-yl)-1H-pyrazol-5-yl]methyl}-8-propyl[1,2,4]triazolo[1,5-c]pyrimidine (184)

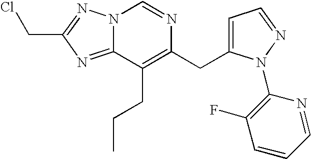

To a solution of (7-{[1-(3-fluoropyridin-2-yl)-1H-pyrazol-5-yl]methyl}-8-propyl[1,2,4]triazolo[1,5-c]pyrimidin-2-yl)methanol (140) (60 mg, 0.16 mmol) in DCM (5 mL) at 0° C. is added SOCl₂ (0.2 mL) under N₂. The mixture is stirred at room temperature for three hours and is poured into saturated NaHCO₃ (10 mL). After CO₂ evolution ceases, the mixture is extracted into DCM, dried (MgSO₄), filtered and solvent evaporated in vacuo. PTLC purification with 5% MeOH/DCM gives pure (184). ¹H NMR δ (CDCl₃) 0.98 (t, 3H), 1.63-1.71 (m, 2H), 2.92 (t, 2H), 4.46 (s, 2H), 4.70 (s, 2H), 6.16 (s, 1H), 7.33-7.41 (m, 1H), 7.64 (t, 1H), 7.70 (s, 1H), 8.34 (d, 1H), 9.07 (s, 1H).

### Step 2. 7-{[1-(3-fluoropyridin-2-yl)-1H-pyrazol-5-yl]methyl}-8-propyl-2-(pyrrolidin-1-ylmethyl) [1,2,4]triazolo[1,5-c]pyrimidine (185)

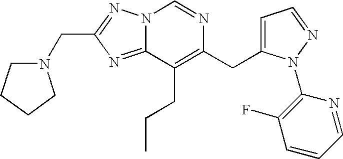

To a solution of 2-(chloromethyl)-7-{[1-(3-fluoropyridin-2-yl)-1H-pyrazol-5-yl]methyl}-8-propyl[1,2,4]triazolo[1,5-c]pyrimidine (184) (40 mg, 0.10 mmol) in acetonitrile (5 mL) is added pyrrolidine (0.2 mL) and K₂CO₃ (43 mg, 0.30 mmol). The mixture is stirred at room temperature overnight. The solvent is removed in vacuo and water (10 mL) and DCM (10 mL) are added to the residue. The layers are separated and the aqueous layer is extracted with DCM (2×10 mL). The combined extracts are washed with brine (15 mL), dried with Na₂SO₄, filtered and solvent evaporated in vacuo. PTLC purification with 5% MeOH/DCM gives the pure title product (185). ¹H NMR δ (CDCl₃) 0.97 (t, 3H), 1.51-1.71 (m, 2H), 1.85 (m, 4H), 2.70 (m, 4H), 2.91 (t, 2H), 3.97 (s, 2H), 4.43 (s, 2H), 6.17 (s, 1H), 7.36 (m, 1H), 7.63 (t, 1H), 7.70 (s, 1H), 8.34 (d, 1H), 9.04 (s, 1H).

The compounds shown in Table 3 are synthesized via similar procedures.

TABLE 3

| | Compound | Name | LC-MS/NMR |
|---|---|---|---|
| 186 | 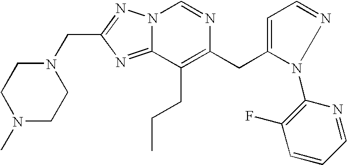 | 7-{[1-(3-fluoropyridin-2- yl)-1H-pyrazol-5- yl]methyl}-2-[(4- methylpiperazin-1- yl)methyl]-8- propyl[1,2,4]triazolo[1,5- c]pyrimidine | ¹H NMR δ (CDCl₃) 0.96 (t, 3H), 1.60-1.70 (m, 2H), 2.29 (s, 3H), 2.40-2.74 (m, 8H), 2.89 (t, 2H), 3.86 (s, 2H), 4.43 (s, 2H), 6.15 (d, 1H), 7.33- 7.40 (m, 1H), |
| #7.63 (t, 1H), 7.65 (d, 1H), 8.33 (d, 1H), 9.04 (s, 1H) | | | |
| 187 | 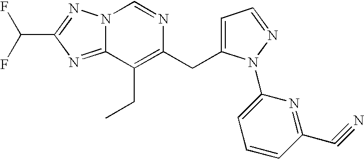 | 6-(5-{[2-(difluoromethyl)- 8-ethyl[1,2,4]triazolo[1,5- c]pyrimidin-7-yl]methyl}- 1H-pyrazol-1-yl)pyridine- 2-carbonitrile | ¹H NMR δ (CDCl₃) 1.37 (t, 3H), 3.16 (q, 2H), 4.88 (s, 2H), 6.09 (d, 1H), 7.08 (t, 1H), 7.54 (d, 1H), 7.66 (d, 1H), 7.94 (t, 1H), 8.31 (d, 1H), 9.15 (s, 1H) |
| 188 | 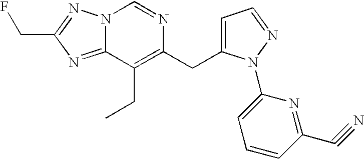 | 6-(5-{[2-(fluoromethyl)-8- propyl[1,2,4]triazolo[1,5- c]pyrimidin-7-yl]methyl}- 1H-pyrazol-1-yl)pyridine- 2-carbonitrile | ¹H NMR δ (CDCl₃) 1.05 (t, 3H), 1.73-1.85 (m, 2H), 3.09 (t, 2H), 4.85 (s, 2H), 5.69 (d, 2H), 6.09 (s, 1H), 7.53 (d, 1H), 7.66 (s, 1H), 7.94 (t, 1H), 8.30 (d, 1H), 9.11 (s, 1H) |
| 189 | 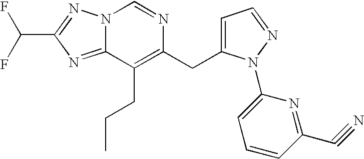 | 6-(5-{[2-(difluoromethyl)-8- propyl[1,2,4]triazolo[1,5- c]pyrimidin-7-yl]methyl}- 1H-pyrazol-1-yl)pyridine- 2-carbonitrile | ¹H NMR δ (CDCl₃) 1.05 (t, 3H), 1.73-1.85 (m, 2H), 3.11 (t, 2H), 4.88 (s, 2H), 6.11 (d, 1H), 6.92 (t, 1H), 7.54 (d, 1H), 7.67 (d, 1H), 7.94 (t, 1H), 8.32 (d, 1H), 9.15 (s, 1H) |
| 190 | 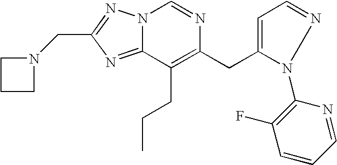 | 2-(azetidin-1-ylmethyl)-7- {[1-(3-fluoropyridin-2-yl)- 1H-pyrazol-5-yl]methyl}-8- propyl[1,2,4]triazolo[1,5- c]pyrimidine | ¹H NMR δ (CDCl₃) 0.95 (t, 3H), 1.58-1.70 (m, 2H), 2.13 (p, 2H), 2.90 (t, 2H), 3,39 (t, 4H) 3.86 (s, 2H), 4.41 (s, 2H), 6.14 (s, 1H), 7.33-7.38 (m, 1H), 7.61 (t, 1H), 7.68 (d, 1H), |
| #8.31 (d, 1H), 9.02 (s, 1H) | | | |
| 191 | 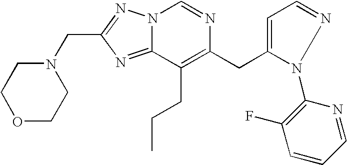 | 7-{[1-(3-fluoropyridin-2- yl)-1H-pyrazol-5- yl]methyl}-2-(morpholin- 4-ylmethyl)-8- propyl[1,2,4]triazolo[1,5- c]pyrimidine | ¹H NMR δ (CDCl₃) 0.96 (t, 3H), 1.56-1.70 (m, 4H), 2.60- 2.66 (m, 4H), 2.89 (t, 2H), 3.80 (s, 2H), 3.77 (t, 3H), 3.84 (s, 2H), 4.43 (s, 2H), 6.15 (d, 1H), 7.33-7.40 (m, 1H), 7.63 |
| # (t, 1H), 7.68 (d, 1H), 8.33 (d, 1H), 9.05 (s, 1H) | | | |
| 192 | 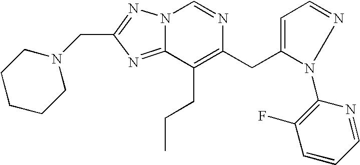 | 7-{[1-(3-fluoropyridin-2- yl)-1H-pyrazol-5- yl]methyl}-2-(piperidin-1- ylmethyl)-8- propyl[1,2,4]triazolo[1,5- c]pyrimidine | ¹H NMR δ (CDCl₃) 0.96 (t, 3H), 1.40-1.48 (m, 2H), 1.60- 1.68 (m, 6H), 2.54 (m, 4H), 2.89 (t, 2H), 3.80 (s, 2H), 4.42 (s, 2H), 6.15 (d, 1H), 7.33- 7.38 (m, 1H), 7.62 (t, 1H), |
| #7.68 (d, 1H), 8.32 (d, 1H), 9.04 (s, 1H) | | | |
| 193 | 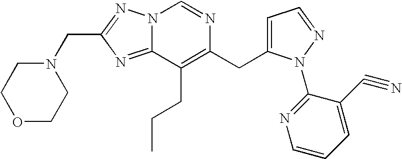 | 2-(5-{[2-(morpholin-4- ylmethyl)-8- propyl[1,2,4]triazolo[1,5- c]pyrimidin-7-yl]methyl}- 1H-pyrazol-1- yl)nicotinonitrile | ¹H NMR (300 MHz, CDCl₃) δ9.05 (1H, s), 8.58 (1H, dd, J =1.8, 4.8 Hz), 8.16 (1H, dd, J =1.8, 7.8 Hz), 7.75 (1H, d, J =1.5 Hz), 7.35 (1H, dd, J = 4.8, 7.8 Hz), 6.18 (1H, |
| # s), 4.67 (2H, s), 3.87 (2H, s), 3.78 (4H, m), 3.00 (2H, m), 2.66 (2H, m), 1.73 (2H, m), 1.42 (2H, dt, J = 3.0, 7.2 Hz), 1.02 (3H, t, J =7.5 Hz) | | | |
| 194 | 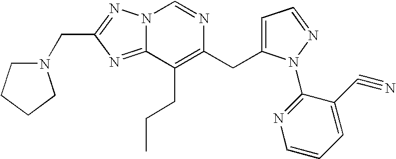 | 2-(5-{[8-propyl-2- (pyrrolidin-1- ylmethyl)[1,2,4]triazolo[1, 5-c]pyrimidin-7- yl]methyl}-1H-pyrazol-1- yl)nicotinonitrile | ¹H NMR (300 MHz, CDCl₃) δ9.05 (1H, s), 8.58 (1H, dd, J =1.8, 4.8 Hz), 8.16 (1H, dd, J =1.8, 7.8 Hz), 7.75 (1H, d, J =1.8 Hz), 7.35 (1H, dd, J = 4.8, 7.5 Hz), 6.18 (1H, |
| # d, J = 1.2 Hz), 4.67 (2H, s), 4.07 (2H, s, br), 3.00 (2H, m), 2.84 (4H, s, br), 1.89 (4H, s, br), 1.73 (2H, m), 1.01 (3H, t, J = 7.5 Hz) | | | |
| 195 | 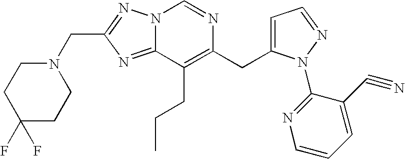 | 2-[5-({2-[(4,4- difluoropiperidin-1- yl)methyl]-8- propyl[1,2,4]triazolo[1,5- c]pyrimidin-7-yl}methyl)- 1H-pyrazol-1- yl]nicotinonitrile | ¹H NMR (300 MHz, CDCl₃) δ9.05 (1H, s), 8.58 (1H, dd, J =1.8, 4.8 Hz), 8.17 (1H, dd, J =2.1, 7.8 Hz), 7.75 (1H, d, J =1.8 Hz), 7.35 (1H, dd, J = 4.8, |
| # 7.8 Hz), 6.18 (1H, s), 4.67 (2H, s), 3.91 (2H, s), 3.00 (2H, m), 2.74 (4H, m), 2.06 (4H, m), 1.73 (2H, m), 1.02 (3H, t, J = 7.5 Hz) | | | |
| 196 | 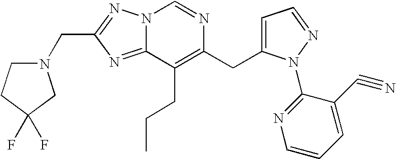 | 2-[5-({2-[(3,3- difluoropyrrolidin-1- yl)methyl]-8- propyl[1,2,4]triazolo[1,5- c]pyrimidin-7-yl}methyl)- 1H-pyrazol-1- yl]nicotinonitrile | ¹H NMR (300 MHz, CDCl₃) δ9.04 (1H, s), 8.58 (1H, dd, J =1.8, 4.8 Hz), 8.16 (1H, dd, J =1.8, 8.1 Hz), 7.75 (1H, d, J =1.5 Hz), 7.34 (1H, dd, J = 4.8, |
| # 7.5 Hz), 6.18 (1H, d, J = 1.8 Hz), 4.67 (2H, s), 4.00 (2H, s), 3.12 (2H, t, J = 13.5 Hz), 2.97 (4H, m), 2.33 (2H, m), 1.74 (2H, m), 1.02 (3H, t, J =7.5 Hz) | | | |
| 197 | 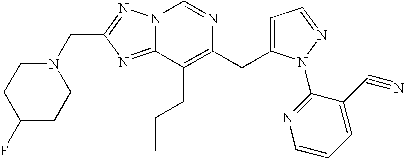 | 2-[5-({2-[(4- fluoropiperidin-1- yl)methyl]-8- propyl[1,2,4]triazolo[1,5- c]pyrimidin-7-yl}methyl)- 1H-pyrazol-1- yl]nicotinonitrile | ¹H NMR (300 MHz, CDCl₃) δ9.05 (1H, s), 8.58 (1H, dd, J =1.8, 4.5 Hz), 8.16 (1H, dd, J =1.8, 7.5 Hz), 7.75 (1H, d, J =1.8 Hz), 7.34 (1H, dd, J = 4.8, |
| # 7.5 Hz), 6.18 (1H, d, J = 1.5 Hz), 4.61˜4.83 (4H, m, overlapped), 3.86 (2H, s), 3.00 (2H, m), 2.74 (2H, m), 2.59 (2H, m), .85˜2.05 (4H, m), 1.73 (2H, m), 1.01 (3H, t, J = 7.2 Hz) | | | |

### I. 7-{[1-(3-fluoropyridin-2-yl)-1H-pyrazol-5-yl]methyl}-8-propyl-2-(1,3-thiazol-2-yl)[1,2,4]triazolo[1,5-c]pyrimidine (201)

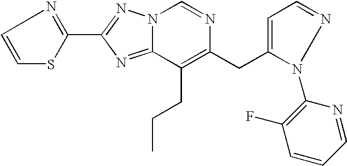

### Step 1. (2-amino-8-propyl-[1,2,4]triazolo[1,5-c]pyrimidin-7-yl)-[2-(3-fluoro-pyrimidin-7-yl)-[2-H-pyrazol-3-yl]acetic acid ethyl ester (198)

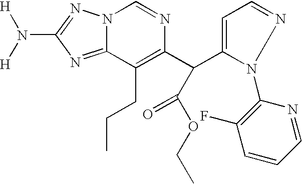

A solution of [2-(3-fluoro-pyridin-2-yl)-2H-pyrazol-3-yl]-(6-hydrazino-5-propyl-pyrimidin-4-yl)-acetic acid ethyl ester (123) (0.65 g) and cyanogen bromide (0.20 g) in EtOH (15 mL) is heated under reflux for two hours. The solvent is removed in vacuo and water (10 mL) and DCM (10 mL) are added to the residue. The layers are separated and the aqueous layer is extracted with DCM (2×10 mL). The combined extracts are washed with brine (15 mL), dried with Na₂SO₄, filtered and solvent evaporated in vacuo. PTLC in 5% MeOH/DCM gives the pure title product 198.

### Step 2. (2-bromo-8-propyl-[1,2,4]triazolo[1,5-c]pyrimidin-7-yl)-[2-(3-fluoro-pyridin-2-yl)-2H-pyrazol-3-yl]-acetic acid ethyl ester (199)

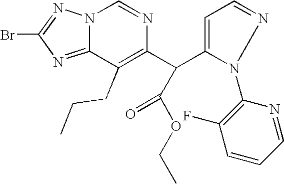

To a solution of (2-amino-5-propyl-[1,2,4]triazolo[1,5-c]pyrimidin-7-yl)-[2-(3-fluoro-pyrimidin-7-yl)-[2-H-pyrazol-3-yl]acetic acid ethyl ester (198) (110 mg) in HBr (2 mL) at 0° C. is added dropwise a solution of sodium nitrite (27 mg) in water (2 mL). The mixture is stirred at 0° C. for 20 minutes. CuBr (56 mg) is added and the resulting mixture is stirred at 0° C. for two hours. NaHCO₃ is added to adjust the pH to over 7. The mixture is extracted with EtOAc (3×20 mL). The combined extracts are washed with brine (15 mL), dried with Na₂SO₄ and filtered, and the solvent is evaporated in vacuo. PTLC in 5% MeOH/DCM gives the pure title product 199.

### Step 3. [2-(3-fluoro-pyridin-2-yl)-2H-pyrazol-3-yl]-(8-propyl-2-thiazol-2-yl-[1,2,4]triazolo[1,5-c]pyrimidin-7-yl)-acetic acid ethyl ester (200)

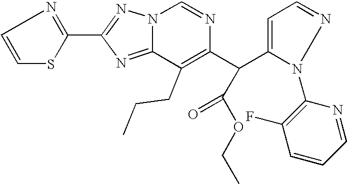

Tributyltinthioazole (57 mg) and Pd(Ph₃P)₄ (20 mg) are added to a solution of (2-bromo-8-propyl-[1,2,4]triazolo[1,5-c]pyrimidin-7-yl)-[2-(3-fluoro-pyridin-2-yl)-2H-pyrazol-3-yl]-acetic acid ethyl ester (199) (50 mg) in toluene (10 mL). The mixture is degassed for 10 minutes, and then heated at 110° C. overnight. The solvent is removed, neutralized with 37% KF solution, and extracted with EtOAc. The combined organic layers are dried, solvent removed to give crude product, which is purified by TLC with 5% MeOH in DCM to give the product 200.

### Step 4. 7-{[1-(3-fluoropyridin-2-yl)-1H-pyrazol-5-yl]methyl}-8-propyl-2-(1,3-thiazol-2-yl)[1,2,4]triazolo[1,5-c]pyrimidine (201)

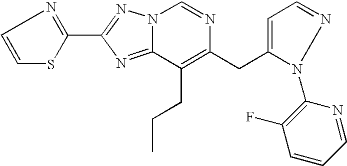

The compound 201 is synthesized via the method provided in Example 2A (step 3) using compound 200 as starting material. ¹H NMR δ (CDCl₃) 1.01 (t, 3H), 1.58-1.74 (m, 2H), 2.98 (t, 2H), 4.48 (s, 2H), 6.21 (s, 1H), 7.33-7.38 (m, 1H), 7.55 (d, 1H), 7.62 (t, 1H), 7.71 (s, 1H), 8.05 (d, 1H), 8.33 (s, 1H), 9.12 (s, 1H).

### J. 2-{5-[(8-PROPYL-2-PYRROLIDIN-1-YL[1,2,4]TRIAZOLO [1,5-C]PYRIMIDIN-7-YL)METHYL]-1H-PYRAZOL-1-YL}NICOTINONITRILE (204)

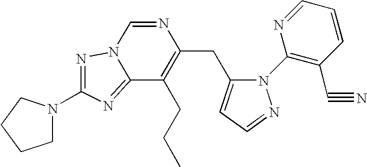

### Step 1. 7-{[1-(3-bromopyridin-2-yl)-1H-pyrazol-5-yl]methyl}-2-chloro-8-propyl[1,2,4]triazolo[1,5-c]pyrimidine (202)

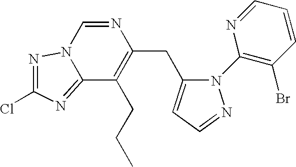

Compound 202 is synthesized via procedures similar to that illustrated by Example 21.

### Step 2. 7-{[1-(3-bromopyridin-2-yl)-1H-pyrazol-5-yl]methyl}-8-propyl-2-pyrrolidin-1-yl[1,2,4]triazolo[1,5-c]pyrimidine (203)

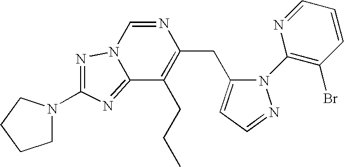

A mixture of 7-{[1-(3-bromopyridin-2-yl)-1H-pyrazol-5-yl]methyl}-2-chloro-8-propyl[1,2,4]triazolo[1,5-c]pyrimidine (202) (42 mg, 0.1 mmol) and pyrrolidine (0.4 ml) is heated at 70° C. in a sealed tube overnight. Excess pyrrolidine is evaporated in vacuo and the residue is purified by preparative TLC with EtOAc to give the title compound as a light yellow solid (203).

### Step 3. 2-{5-[(8-propyl-2-pyrrolidin-1-yl[1,2,4]triazolo[1,5-c]pyrimidin-7-yl)methyl]-1H-pyrazol-1-yl}nicotinonitrile (204)

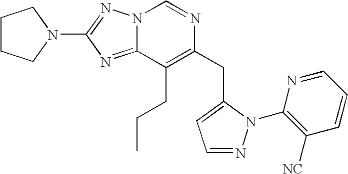

Compound 204 is synthesized via a procedure similar to that illustrated by Example 1D (step 3) using compound 203 as a starting material. ¹H NMR (CDCl₃, δ): 8.80 (s, 1H), 8.56 (dd, 1H), 8.14 (dd, 1H), 7.74 (d, 1H), 7.32 (dd, 1H), 6.14 (d, 1H), 4.58 (s, 2H), 3.54-3.58 (m, 4H), 2.85-2.90 (m, 2H), 1.98-2.03 (M, 4H), 1.65-1.77 (m, 2H), 0.99 (t, 3H).

Compounds 205, 206 and 207 are synthesized via a similar procedure.

### 2-{5-[(2-PIPERIDIN-1-YL-8-PROPYL[1,2,4]TRIAZOLO [1,5-C]PYRIMIDIN-7-YL)METHYL]-1H-PYRAZOL-1-YL}NICOTINONITRILE (205)

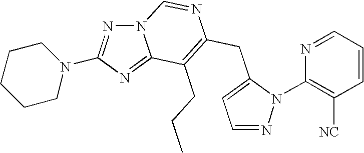

¹H NMR (CDCl₃, δ): 8.76 (s, 1H), 8.56 (dd, 1H), 8.14 (dd, 1H), 7.74 (d, 1H), 7.31 (dd, 1H), 6.14 (d, 1H), 4.57 (s, 2H), 3.60 (s, 4H), 2.83-2.88 (m, 2H), 1.60-1.74 (m, 8H), 0.99 (t, 3H).

### 2-{5-[(2-MORPHOLIN-4-YL-8-PROPYL[1,2,4]TRIAZOLO [1,5-C]PYRIMIDIN-7-YL)METHYL]-1H-PYRAZOL-1-YL}NICOTINONITRILE (206)

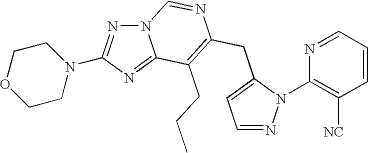

¹H NMR (CDCl₃, δ): 8.78 (s, 1H), 8.56 (dd, 1H), 8.14 (dd, 1H), 7.73 (d, 1H), 7.32 (dd, 1H), 6.14 (d, 1H), 4.59 (s, 2H), 3.81 (t, 4H), 3.62 (t, 4H), 2.84-2.88 (m, 2H), 1.65-1.75 (m, 2H), 0.99 (t, 3H).

### 2-{5-[(2-(2,6-DIMETHYLMORPHOLIN-4-YL)-8-PROPYL[1,2,4]TRIAZOLO[1,5-C]PYRIMIDIN-7-YL)METHYL]-1H-PYRAZOL-1-YL}NICOTINONITRILE (207)

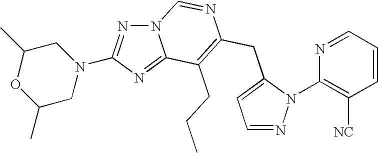

¹H NMR (CDCl₃, δ): 8.77 (s, 1H), 8.57 (dd, 1H), 8.14 (dd, 1H), 7.74 (d, 1H), 7.32 (dd, 1H), 6.15 (d, 1H), 4.59 (s, 2H), 4.08 (dd, 2H), 3.69-3.80 (m, 2H), 2.84-2.89 (m, 2H), 2.70 (dd, 2H), 1.67-1.74 (m, 2H), 1.26 (d, 6H), 0.99 (t, 3H).

### K. 2-{5-[(2-METHOXY-8-PROPYL[1,2,4]TRIAZOLO[1,5-C]PYRIMIDIN-7-YL)METHYL]-1H-PYRAZOL-1-YL}NICOTINONITRILE (209)

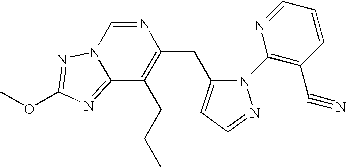

### Step 1. 7-{[1-(3-bromopyridin-2-yl)-11H-pyrazol-5-yl]methyl}-2-methoxy-8-propyl[1,2,4]triazolo[1,5-c]pyrimidine (208)

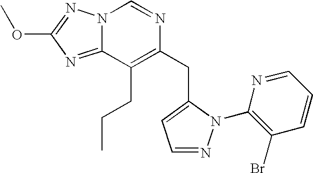

A mixture of 7-{[1-(3-bromopyridin-2-yl)-1H-pyrazol-5-yl]methyl}-2-chloro-8-propyl[1,2,4]triazolo[1,5-c]pyrimidine (202) (67 mg, 0.16 mmol) and NaOMe (0.5 N in MeOH, 1 ml, 0.5 mmol) in MeOH (5 ml) is heated in a sealed tube at 80° C. for 6 hours. The solvent is removed in vacuo and the residue is purified by preparative TLC with EtOAc gives the title compound as a light yellow solid (208).

### Step 2. 2-{5-[(2-methoxy-8-propyl[1,2,4]triazolo[1,5-c]pyrimidin-7-yl)methyl]-1H-pyrazol-1-yl}nicotinonitrile (209)

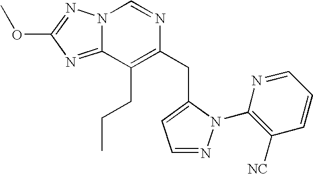

Compound 209 is synthesized via a procedure similar to that illustrated by Example 1D (step 3) using compound 208 as a starting material. ¹H NMR (CDCl₃, δ): 8.86 (s, 1H), 8.56 (dd, 1H), 8.15 (dd, 1H), 7.50 (d, 1H), 7.33 (dd, 1H), 6.18 (d, 1H), 4.64 (s, 2H), 4.12 (s, 3H), 2.88-2.94 (m, 2H), 1.65-1.75 (m, 2H), 1.01 (t, 3H).

### L. 7-{[1-(3-CYANOPYRIDIN-2-YL)-1H-PYRAZOL-5-YL]METHYL}-8-PROPYL[1,2,4]TRIAZOLO[1,5-C]PYRIMIDINE-2-CARBONITRILE (210)

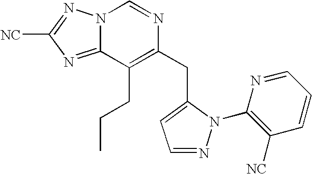

Compound 210 is synthesized via a procedure similar to that illustrated by Example 1D (step 3) using compound 202 as a starting material. ¹H NMR (CDCl₃, 8): 9.13 (s, 1H), 8.55 (dd, 1H), 8.17 (dd, 1H), 7.76 (d, 1H), 7.34 (dd, 1H), 6.20 (d, 1H), 4.69 (s, 2H), 2.95-3.00 (m, 2H), 1.70-1.75 (m, 2H), 1.03 (t, 3H). 2-{5-[(2-Chloro-8-propyl[1,2,4]triazolo[1,5-c]pyrimidin-7-yl)methyl]-1H-pyrazol-1-yl}nicotinonitrile (211) is also obtained.

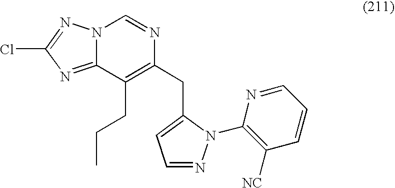

¹H NMR (CDCl₃, δ): 8.98 (s, 1H), 8.55 (dd, 1H), 8.17 (dd, 1H), 7.76 (d, 1H), 7.34 (dd, 1H), 6.20 (d, 1H), 4.69 (s, 2H), 2.95-3.00 (m, 2H), 1.70-1.75 (m, 2H), 1.03 (t, 3H).

### M. 7-{[1-(6-fluoropyridin-2-yl)-1H-pyrazol-5-yl]oxy}-8-propyl[1,2,4]triazolo[1,5-c]pyrimidine (212)

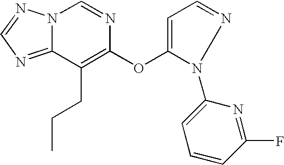

A mixture of 4-{[1-(6-fluoropyridin-2-yl)-1H-pyrazol-5-yl]oxy}-6-iodo-5-propylpyrimidine (116) (0.37 g) and hydrazine (84 mg) in EtOH (10 mL) is heated at 60° C. overnight. The solvent is removed in vacuo and formic acid (5 mL) is added. The mixture is stirred at 110° C. for three hours. Formic acid is removed in vacuo and to the residue is added NaHCO₃ (aq.) (10 mL) and DCM (20 mL). The organic layer is separated and the aqueous layer is extracted with DCM (2×20 mL). The combined organic layers are dried (NaSO₄) and solvent is removed. The crude product is separated by column chromatography (5% MeOH in DCM) to give 212. ¹H NMR δ (CDCl₃) 1.06 (t, 3H), 1.84-1.94 (m, 2H), 3.11 (t, 2H), 6.23 (d, 1H), 6.71 (dd, 1H), 7.72-7.90 (m, 3H), 8.35 (s, 1H), 8.93 (s, 1H).

### Example 3

### Synthesis of IMIZAZO[1,2-C]PYRIMIDINES

### A. 7-{[1-(3-fluoropyridin-2-yl)-1H-pyrazol-5-yl]methyl}-8-propylimidazo[1,2-c]pyrimidine (215)

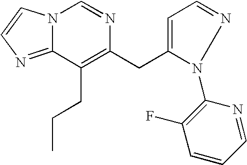

### Step 1. [2-(3-fluoro-pyridin-2-yl)-2H-pyrazol-3-yl]-(6-azido-5-propyl-pyrimidin-4-yl)-acetic acid ethyl ester (213)

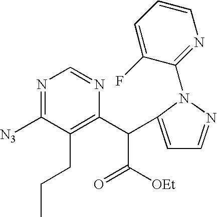

A solution of [2-(3-fluoro-pyridin-2-yl)-2H-pyrazol-3-yl]-(6-iodo-5-propyl-pyrimidin-4-yl)acetic acid ethyl ester (104) (60 mg) and NaN₃ (50 mg) in DMF (2 mL) is heated at 70° C. in a sealed tube overnight. The solvent is removed in vacuo and water (10 mL) and EtOAc (10 mL) are added to the residue. The layers are separated and the aqueous layer is extracted with EtOAc (2×10 mL). The combined extracts are washed with brine (15 mL) and dried with Na₂SO₄. The solvent is removed in vacuo and the resulting yellow oil (213) is used in the next step without further purification.

### Step 2. [2-(3-fluoro-pyridin-2-yl)-2H-pyrazol-3-yl]-(6-amino-5-propyl-pyrimidin-4-yl)-acetic acid ethyl ester (214)

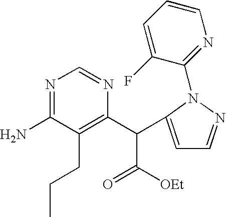

Pd/C (10%, 10 mg) is added to a solution of [2-(3-fluoro-pyridin-2-yl)-2H-pyrazol-3-yl]-(6-azido-5-propyl-pyrimidin-4-yl)-acetic acid ethyl ester (213) (60 mg) in EtOH (20 mL). The mixture is stirred under H₂ at 30 psi overnight. The catalyst is removed by filtration and the filtrate is evaporated in vacuo. The resulting light yellow solid (214) is used in the next step without further purification.

### Step 3. 7-{[1-(3-fluoropyridin-2-yl)-1H-pyrazol-5-yl]methyl}-8-propylimidazo[1,2-c]pyrimidine (215)

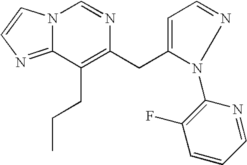

A solution of [2-(3-fluoro-pyridin-2-yl)-2H-pyrazol-3-yl]-(6-amino-5-propyl-pyrimidin-4-yl)acetic acid ethyl ester (214) (20 mg) and chloroacetaldehyde (1 mL) in DMF (5 mL) is heated overnight at 70° C. in a sealed tube. The solvent is removed in vacuo and 6N HCl (2 mL) is added. The resulting mixture is heated at 60° C. for 3 hours, then neutralized with saturated NaHCO₃. The aqueous solution is extracted with DCM (2×15 mL). The combined extracts are washed with brine (15 mL), dried (Na₂SO₄) and evaporated. PTLC separation of the residue with 5% MeOH in CH₂Cl₂ provides a white solid (215). ¹H NMR δ (CDCl₃) 0.99 (t, 3H), 1.60-1.67 (m, 2H), 2.90 (t, 2H), 4.36 (s, 2H), 6.15 (s, 1H), 7.34-7.42 (m, 1H), 7.54-7.70 (m, 4H), 8.36 (d, 1H), 8.78 (s, 1H).

### Example 4

### Synthesis of 7-{[1-(3-FLUOROPYRIDIN-2-YL)-1H-PYRAZOL-5-YL]METHYL}-1-METHYL-8-PROPYLIMIDAZO[1,5-c]PYRIMIDINE (217)

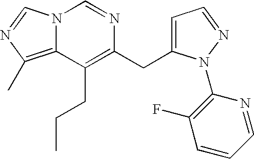

### Step 1. 1-(6-{[1-(3-Fluoropyridin-2-yl)-1H-pyrazol-5-yl]methyl}-5-propylpyrimidin-4-yl)ethanone (216)

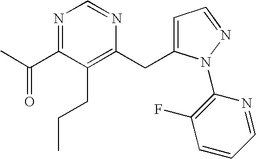

Tributyltinvinylethylether (0.186 g) and Pd(Ph₃P)₄ (40 mg) are added to a solution of [2-(3-fluoro-pyridin-2-yl)-2H-pyrazol-3-yl]-(6-iodo-5-propyl-pyrimidin-4-yl)-acetic acid ethyl ester (104) (0.17 g) in toluene (30 mL). The mixture is degassed for 10 minutes, and then heated at 110° C. overnight. The solvent is removed under vacuum to obtain the crude product, which is then dissolved in 6N of HCl (10 mL) and the mixture is stirred at 60° C. for 3 hours. The solvent is removed, and the residue is neutralized with saturated NaHCO₃ and extracted with EtOAc. The combined organic layers are dried and solvent removed to give crude product, which is purified by TLC with 5% MeOH in DCM to give 216. ¹H NMR δ (CDCl₃) 0.95 (t, 3H), 1.44-1.55 (m, 2H), 2.64 (s, 3H), 2.74 (t, 2H), 4.52 (s, 2H), 6.17 (s, 1H), 7.31-7.37 (m, 1H), 7.62 (t, 1H), 7.71 (s, 1H), 8.24 (d, 1H), 8.91 (s, 1H).

### Step 2. 7-{[1-(3-fluoropyridin-2-yl)-1H-pyrazol-5-yl]methyl}-1-methyl-8-propylimidazo[1,5-c]pyrimidine (217)

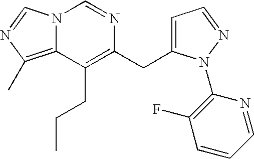

A mixture of 1-(6-{[1-(3-fluoropyridin-2-yl)-1H-pyrazol-5-yl]methyl}-5-propylpyrimidin-4-yl)ethanone (216) (60 mg) and formic acid (0.1 mL) is added to 2 mL of formamide at 160-180° C. The mixture is heated at 160-180° C. for an additional 3 hours. During this period, additional formic acid (0.2 mL) is added. The mixture is cooled to room temperature and poured into water (10 mL). The solution is made alkaline to at least pH 11 with concentrated NaOH. The solution is extracted with EtOAc. The combined organic layers are dried over MgSO₄, and the solvent is removed to give the crude product, which is heated with POCl₃ (1 mL) at reflux for 3 hours. Excess POCl₃ is removed, EtOAc (10 mL) is added, and the mixture is washed with saturated NaHCO₃ (5 mL) and brine (5 mL), and dried over MgSO₄. After evaporation of the solvent, the residue is purified by PTLC with 5% MeOH in DCM to give the title product 217. ¹H NMR δ (CDCl₃) 1.01 (t, 3H), 1.52-1.63 (m, 2H), 2.59 (s, 3H), 2.71 (t, 2H), 4.19 (s, 2H), 6.13 (s, 1H), 7.38 (s, 1H), 7.62 (t, 1H), 7.68 (s, 1H), 8.12 (s, 1H), 8.37 (s, 1H), 8.51 (s, 1H).

### Example 5

### Synthesis of Pyrazines

### A. 2-{5-[(3-PROPYLPYRAZIN-2-YL)METHYL]-1H-PYRAZOL-1-YL}NICOTINONITRILE (307)

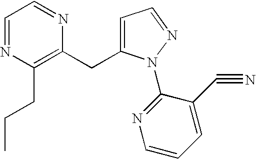

Step 1. 3-chloro-2-propyl-pyrazine (300)

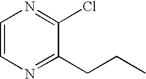

A mixture of 3-propyl-pyrazin-2-ol (50 g) and POCl₃ (50 mL) is heated at reflux for 2 hours. The solvent is removed in vacuo and EtOAc (30 mL) and water (30 mL) are added to the residue. NaHCO₃ is carefully added until the pH of the aqueous layer is greater than 7. The layers are separated and the aqueous layer is extracted with EtOAc (2×30 mL). The combined extracts are washed with brine (50 mL) and dried (Na₂SO₄), and solvent is evaporated. Flash column purification of the residue (EtOAc:hexane=6:1) provides the product as a light yellow oil (300). ¹H NMR δ (CDCl₃) 1.02 (t, 3H), 1.80 (h, 2H), 2.93 (t, 2H), 8.19 (d, 1H), 8.41 (d, 1H).

### Step 2. 3-chloro-2-propyl-pyrazin-1-ol (301)

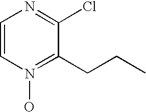

A mixture of 3-chloro-2-propyl-pyrazine (300) (4.3 g) and MCPBA (7.38 g) in 1,2-dichloroethane is heated at 65° C. overnight. Ammonia is passed through the solution until no more precipitate is formed. The solid is filtered and washed with DCM. Concentration of the filtrate gives 301. ¹H NMR δ (CDCl₃) 1.04 (t, 3H), 1.63-1.76 (m, 2H), 3.02 (t, 2H), 8.04 (d, 1H), 8.08 (d, 1H).

### Step 3. [2-(3-bromo-pyridin-2-yl)-2H-pyrazol-3-yl]-(4-hydroxy-3-propyl-pyrazin-2-yl)-acetic acid ethyl ester (302)

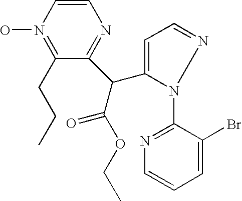

NaH (0.77 g, 60% in mineral oil) is added to a solution of [2-(3-bromo-pyridin-2-yl)-2H-pyrazol-3-yl]-acetic acid ethyl ester (5 g) in DMSO (25 mL) at room temperature. The mixture is stirred for 10 minutes. 3-Chloro-2-propyl-pyrazin-1-ol (301) (3 g) is then added to the above mixture. The resulting mixture is heated at 110° C. overnight. The mixture is cooled to room temperature. EtOAc (20 mL) and water (20 mL) are added to the residue. The layers are separated and the aqueous layer is extracted with EtOAc (2×20 mL). The combined extracts are washed with brine (10 mL), dried (Na₂SO₄), and solvent evaporated. Flash column purification of the residue (EtOAc:hexane=1:1) provides the product 302.

### Step 4. 3-[2-(3-bromo-pyridin-2-yl)-2H-pyrazol-3-ylmethyl]-2-propyl-pyrazin-1-ol (303)

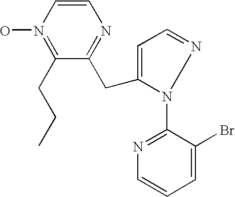

A mixture of [2-(3-bromo-pyridin-2-yl)-2H-pyrazol-3-yl]-(4-hydroxy-3-propyl-pyrazin-2-yl)acetic acid ethyl ester (302) (3 g) in 6N HCl (30 mL) is heated at 70° C. for 6 hours. The reaction mixture is cooled to room temperature, neutralized with saturated NaHCO₃, and extracted with DCM. The solution is dried, and evaporated. The residue is purified by column with 5% MeOH in DCM to give 303. ¹H NMR δ (CDCl₃) 0.95 (t, 3H), 1.47-1.56 (m, 2H), 2.79 (t, 2H), 4.29 (s, 2H), 6.18 (d, 1H), 7.26 (dd, 1H), 7.68 (d, 1H), 7.95 (d, 1H), 8.05 (d, 1H), 8.08 (d, 1H), 8.47 (dd, 1H).

### Step 5. 2-{5-[(4-oxido-3-propylpyrazin-2-yl)methyl]-1H-pyrazol-1-yl}nicotinonitrile (304)

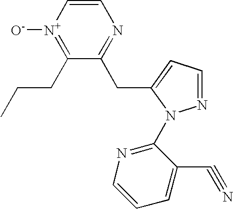

To a solution of 3-[2-(3-bromo-pyridin-2-yl)-2H-pyrazol-3-ylmethyl]-2-propyl-pyrazin-1-ol (303) (1.2 g) and Zn(CN)₂ (0.38 g) in DMF (15 mL), DPPF (0.29 g) and Pd₂(dba)₃ (0.17 g) are added. The mixture is degassed for 10 minutes and is heated at 110° C. overnight. The solvent is removed in vacuo and EtOAc (10 mL) and water (10 mL) are added to the residue. The layers are separated and the aqueous layer is extracted with EtOAc (2×10 mL). The combined extracts are washed with brine (10 mL) and dried (Na₂SO₄), and solvent is evaporated. Column purification of the residue with 5% MeOH in DCM provides the product 304. ¹H NMR δ (CDCl₃) 1.03 (t, 3H), 1.62-1.78 (m, 2H), 2.94 (t, 2H), 4.68 (s, 2H), 6.22 (d, 1H), 7.31 (dd, 1H), 7.77 (d, 1H), 7.95 (d, 1H), 8.07 (d, 1H), 8.14 (dd, 1H), 8.47 (dd, 1H).

### Step 6. 2-[5-(5-chloro-3-propyl-pyrazin-2-ylmethyl)-pyrazol-1-yl]-nicotinonitrile (305)

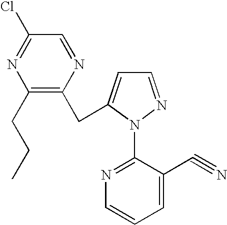

A mixture of 2-{5-[(4-oxido-3-propylpyrazin-2-yl)methyl]-1H-pyrazol-1-yl}nicotinonitrile (304) (0.2 g) and POCl₃ (5 mL) is heated at 100° C. for 2 hours. The solvent is removed in vacuo and EtOAc (10 mL) and water (10 mL) are added to the residue. NaHCO₃ is carefully added until the pH of aqueous layer is greater than 7. The layers are separated and the aqueous layer is extracted with EtOAc (2×10 mL). The combined extracts are washed with brine (10 mL) and dried (Na₂SO₄), and solvent is evaporated. Flash column purification of the residue (EtOAc:hexane=2:1) provides the title product 305. ¹H NMR δ (CDCl₃) 0.95 (t, 3H), 1.58-1.65 (m, 2H), 2.62 (t, 2H), 4.26 (s, 2H), 6.11 (d, 1H), 7.31 (dd, 1H), 7.66 (d, 1H), 7.87 (d, 1H), 8.18 (d, 1H), 8.41 (dd, 1H).

2-{5-[3-(1-Chloro-propyl)-pyrazin-2-ylmethyl]-pyrazol-1yl-}-nicotinonitrile (306) is also obtained. ¹H NMR δ (CDCl₃) 0.99 (t, 3H), 2.30 (p, 2H), 4.79 (q, 2H), 5.11 (t, 1H), 6.16 (dd, 1H), 7.30 (dd, 1H), 7.75 (d, 1H), 8.13 (dd, 1H), 8.32 (d, 1H), 8.43 (dd, 1H), 8.51 (dd, 1H).

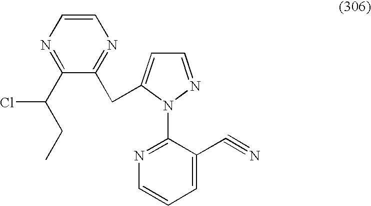

### Step 7. 2-{5-[(3-propylpyrazin-2-yl)methyl]-1H-pyrazol-1-yl}nicotinonitrile (307)

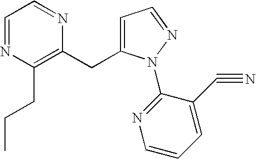

Pd/C (10%, 10 mg) is added to a solution of 2-[5-(5-chloro-3-propyl-pyrazin-2-ylmethyl)pyrazol-1-yl]-nicotinonitrile (305) (10 mg) in EtOH (10 mL). The mixture is stirred under H₂ for 2 hours. The catalyst is removed by filtration and the filtrate is evaporated in vacuo. The residue is purified by PTLC with 5% MeOH in DCM to give the title product 307. ¹H NMR δ (CDCl₃) 0.99 (t, 3H), 1.77 (h, 2H), 2.81 (t, 2H), 4.68 (s, 2H), 6.13 (s, 1H), 7.30 (dd, 1H), 7.75 (d, 1H), 7.83 (dd, 1H), 8.19 (d, 1H), 8.31 (dd, 1H), 8.48 (dd, 1H).

1-(2-{5-[(3-Propylpyrazin-2-yl)methyl]-1H-pyrazol-1-yl}pyridin-3-yl)methanamine (308) is also obtained. ¹H NMR δ (CDCl₃) 0.97 (t, 3H), 1.71 (h, 2H), 2.77 (t, 2H), 4.04 (s, 2H), 4.58 (s, 2H), 6.21 (s, 1H), 7.31 (d, 1H), 7.67 (m, 1H), 8.11 (m, 2H), 8.26 (m, 1H), 8.39 (m, 1H).

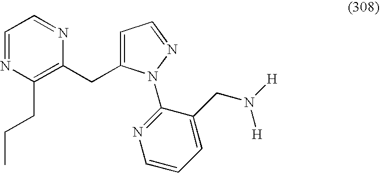

### B. 2-{5-[(5-METHOXY-3-PROPYLPYRAZIN-2-YL)METHYL]-1H-PYRAZOL-1-YL}NICOTINONITRILE (311)

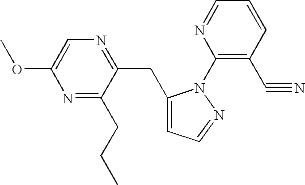

### Step 1. 2-[2-(3-chloro-pyridin-2-yl)-2H-pyrazol-3-ylmethyl]-5-chloro-3-propyl-pyrazine (309)

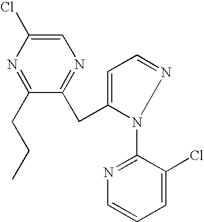

This compound is synthesized via a procedure similar to that illustrated in Example 5A.

### Step 2. 2-[2-(3-Chloro-pyridin-2-yl)-2H-pyrazol-3-ylmethyl]-5-methoxy-3-propyl-pyrazine (310)

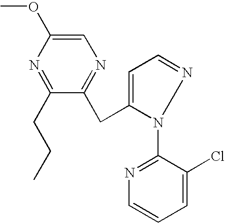

To a solution of 2-[2-(3-chloro-pyridin-2-yl)-2H-pyrazol-3-ylmethyl]-5-chloro-3-propyl-pyrazine (309) (68 mg) in MeOH (5 mL), NaOMe in MeOH (0.1 mL, 25%) is added. The resulting mixture is heated at 60° C. for 3 hours. The solvent is removed in vacuo, and EtOAc (10 mL) and water (10 mL) are added to the residue. The layers are separated and the aqueous layer is extracted with EtOAc (2×10 mL). The combined extracts are washed with brine (10 mL), dried (Na₂SO₄), and solvent evaporated. PTLC of the residue with 5% MeOH in DCM provides the product 310.

### Step 3. 2-{5-[(5-methoxy-3-propylpyrazin-2-yl)methyl]-1H-pyrazol-1-yl}nicotinonitrile (311)

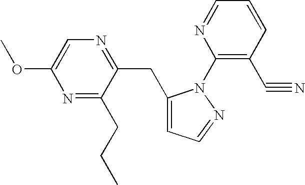

The desired compound 311 is synthesized via a similar procedure illustrated by Example 1D (step 3) using compound 310 as a starting material. ¹H NMR δ (CDCl₃) 0.95 (t, 3H), 1.72 (h, 2H), 2.67 (t, 2H), 3.92 (s, 3H), 4.54 (s, 2H), 6.02 (d, 1H), 7.32 (dd, 1H), 7.70 (d, 1H), 7.87 (dd, 1H), 8.14 (dd, 1H), 88.58 (dd, 1H).

### C. 2-{5-[(5-isopropoxy-3-propylpyrazin-2-yl)methyl]-1H-pyrazol-1-yl}nicotinonitrile (312)

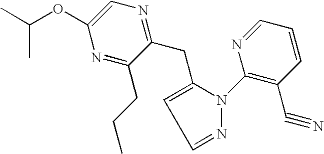

Compound 312 is synthesized following the synthetic procedure of 311 using sodium isopropoxide. ¹H NMR δ (CDCl₃) 0.95 (t, 3H), 1.32 (s, 3H), 1.34 (s, 3H), 1.71 (h, 2H), 2.64 (t, 2H), 4.53 (s, 2H), 5.24 (h, 1H), 6.02 (d, 1H), 7.33 (dd, 1H), 7.70 (d, 1H), 7.80 (d, 1H), 8.14 (d, 1H), 8.59 (dd, 1H).

### D. 2-{5-[(3-PROPYL-5-PYRIDIN-3-YLPYRAZIN-2-YL)METHYL]-1H-PYRAZOL-1-YL}NICOTINONITRILE (313)

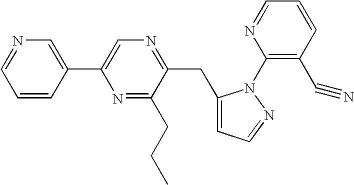

To a solution of 2-[5-(5-chloro-3-propyl-pyrazin-2-ylmethyl)-pyrazol-1-yl]-nicotinonitrile (305) (35 mg) and 3-pyridinoborobic acid (20 mg) in dioxane (5 mL) and water (1 mL), Na₂CO₃ (50 mg) and Pd (PPh₃)₄ (15 mg) are added. The mixture is degassed for 10 minutes and heated at 60° C. for 2 hours. The solvent is removed in vacuo, and EtOAc (10 mL) and water (10 mL) are added to the residue. The layers are separated and the aqueous layer is extracted with EtOAc (2×10 mL). The combined extracts are washed with brine (10 mL), dried (Na₂SO₄), and solvent evaporated. PTLC of the residue (EtOAc:hexane=2:1) provides the product 313. ¹H NMR δ (CDCl₃) 1.03 (t, 3H), 1.85 (h, 2H), 2.88 (t, 2H), 4.72 (s, 2H), 6.17 (d, 1H), 7.29 (dd, 1H), 7.41 (m, 1H), 7.76 (d, 1H), 8.14 (dd, 1H), 8.28 (d, 1H), 8.51 (dd, 1H), 8.65 (s, 2H), 9.22 (s, 1H).

Compounds shown in Table 4 are synthesized via a procedure similar to that described in Example 5.

TABLE 4

| | Compound | Name | LC-MS/NMR |
|---|---|---|---|
| 314 | 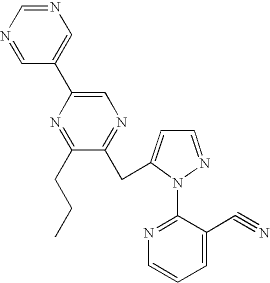 | 2-{5-[(3-propyl-5- pyrimidin-5-ylpyrazin-2- yl)methyl]-1H-pyrazol-1- yl}nicotinonitrile | ¹H NMR δ (CDCl₃) 1.03 (t, 3H), 1.83 (h, 2H), 2.90 (t, 2H), 4.74 (s, 2H), 6.19 (d, 1H), 7.30 (dd, 1H), 7.77 (d, 1H), 8.14 (dd, 1H), 8.49 (dd, 1H), 8.67 (s, 1H), 9.27 (s, 1H), 9.32 (s, 1H). |
| 315 | 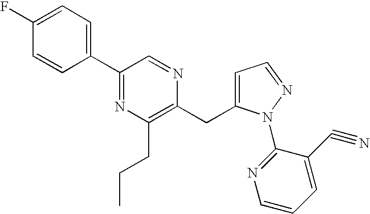 | 2-(5-{[5-(4-fluorophenyl)- 3-propylpyrazin-2- yl]methyl}-1H-pyrazol-1- yl)nicotinonitrile | ¹H NMR δ (CDCl₃) 1.02 (t, 3H), 1.83 (h, 2H), 2.85 (t, 2H), 4.69 (s, 2H), 6.16 (d, 1H), 7.10-7.18 (m, 2H), 7.29 (dd, 1H), 7.77 (d, 1H), 7.94-8.04 (m, 2H), 8.13 (dd, 1H), 8.51 (dd, 1H), 8.59 (s, 1H). |
| 316 | 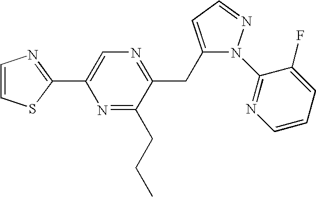 | 2-{[1-(3-fluoropyridin-2- yl)-1H-pyrazol-5- yl]methyl}-3-propyl-5- (1,3-thiazol-2-yl)pyrazine | ¹H NMR δ (CDCl₃) 9.01 (s, 1H), 8.30 (d, 1H), 7.93 (d, 1H), 7.70 (s, 1H), 7.65-7.58 (m, 1H), 7.45 (d, 1H), 7.37-7.32 (m, 1H), 6.12 (s, 1H), 4.49 (s, 2H), 2.76 (t, 2H), 1.80-1.72 (m, 2H), 0.98 (t, 3H). |
| 317 | 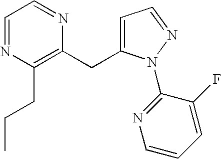 | 2-{[1-(3-fluoropyridin-2- yl)-1H-pyrazol-5- yl]methyl}-3- propylpyrazine | LC-MS (M + 1) 298.31 |
| 318 | 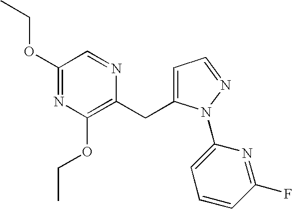 | 3,5-diethoxy-2-{[1-(6- fluoropyridin-2-yl)-1H- pyrazol-5- yl]methyl}pyrazine | ¹H NMR δ (CDCl₃) 7.87-7.80 (m, 2H), 7.61-7.58 (m, 2H), 6.76-6.73 (m, 2H), 6.07 (s, 1H), 4.64 (s, 2H), 4.64-4.27 (m, 4H), 1.43-1.31 (m, 6H). |

### E. 5-{[1-(3-cyanopyridin-2-yl)-1H-pyrazol-5-yl]methyl}-6-propylpyrazine-2-carbonitrile (319)

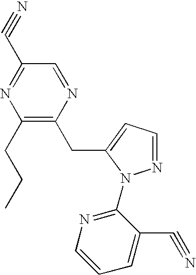

To a solution of 2-[2-(3-chloro-pyridin-2-yl)-2H-pyrazol-3-ylmethyl]-5-chloro-3-propyl)pyrazine (309) (100 mg) and Zn(CN)₂ (50 mg) in DMF (5 mL), DPPF (50 mg) and Pd₂(dba)₃ (50 mg) are added. The mixture is degassed for 10 minutes and heated at 110° C. overnight. The solvent is removed in vacuo and EtOAc (10 mL) and water (10 mL) are added to the residue. The layers are separated and the aqueous layer is extracted with EtOAc (2×10 mL). The combined extracts are washed with brine (10 mL) and dried (Na₂SO₄), and solvent is evaporated. TLC with 5% MeOH in DCM provides the product 319. ¹H NMR δ (CDCl₃) 0.77 (t, 3H), 1.50-1.66 (m, 2H), 2.66-2.87 (m, 2H), 4.58 (s, 2H), 5.89 (s, 1H), 7.53 (m, 1H), 7.97 (s, 1H), 8.20 (d, 1H), 8.90 (s, 2H).

### F. 2-{5-[(3-propenyl)-pyrazin-2-ylmethyl]-pyrazol-1-YL}-nicotinitrile (320)

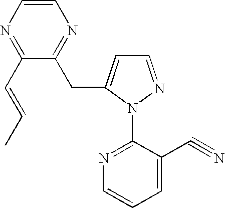

To a solution of 2-[2-(3-chloro-pyridin-2-yl)-2H-pyrazol-3-ylmethyl]-5-chloro-3-(1-chloropropyl)-pyrazine (100 mg) and Zn(CN)₂ (30 mg) in DMF (5 mL), DPPF (20 mg) and Pd₂(dba)₃ (20 mg) are added. The mixture is degassed for 10 minutes and heated at 110° C. overnight. The solvent is removed in vacuo and EtOAc (10 mL) and water (10 mL) are added to the residue. The layers are separated and the aqueous layer is extracted with EtOAc (2×10 mL). The combined extracts are washed with brine (10 mL) and dried (Na₂SO₄), and solvent is evaporated. TLC with 5% MeOH in DCM provides the product 320. ¹H NMR δ (CDCl₃) 1.95 (dd, 3H), 4.68 (s, 2H), 6.10 (d, 1H), 6.64 (dq, 1H), 6.93-7.02 (m, 1H), 7.25-7.31 (m, 1H), 7.72 (d, 1H), 8.12 (dd, 1H), 8.18 (d, 1H), 8.30 (dd, 1H), 8.51 (dd, 1H).

### G. 3-Propyl-5-(1,3-thiazol-2-yl)-2-({1-[3-(1,3-thiazol-2-yl)pyridin-2-yl]-1H-pyrazol-5-yl}methyl)pyrazine (321)

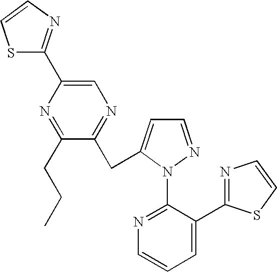

Tributyltinthioazole (30 mg) and Pd(Ph₃P)₄ (15 mg) are added to a solution of 2-[5-(5-chloro-3-propyl-pyrazin-2-ylmethyl)-pyrazol-1-yl]-nicotinonitrile (305) (20 mg) in toluene (10 mL). The mixture is degassed for 10 minutes, and then heated at 110° C. overnight. The solvent is removed, neutralized with a 37% KF solution, and extracted with EtOAc. The combined organic layers are dried, and solvent is removed to give crude product, which is purified by TLC with 5% MeOH in DCM to give the product 321. ¹H NMR δ (CDCl₃) 0.95 (t, 3H), 1.70 (h, 2H), 2.66 (t, 2H), 4.29 (s, 2H), 6.22 (s, 1H), 7.26 (s, 1H), 7.32 (d, 1H), 7.44 (d, 1H), 7.53 (dd, 1H), 7.68 (s, 1H), 7.81 (d, 1H), 7.92 (d, 1H), 8.59 (dd, 1H), 8.67 (dd, 1H), 8.87 (s, 1H).

### Example 6

### Synthesis of TRIAZOLO[4,3-A]PYRAZINES

### A. 2-{5-[(5-propyl[1,2,4]triazolo[4,3-a]pyrazin-6-yl)methyl]-1H-pyrazol-1-yl}nicotinonitrile (324)

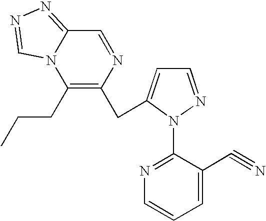

### Step 1. 2-[2-(3-chloro-pyridin-2-yl)-2H-pyrazol-3-ylmethyl]-5-methyl-3-propyl-pyrazine (322)

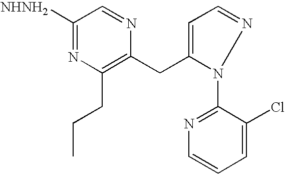

A mixture of hydrazine monohydrate (50 mg) and 2-[2-(3-chloro-pyridin-2-yl)-2H-pyrazol-3-ylmethyl]-5-chloro-3-propyl-pyrazine (309) (0.16 g) in EtOH (10 mL) is heated in a sealed tube at 70° C. overnight. The solvent is removed in vacuo and the residue is triturated with EtOAc and ethyl ether. Filtration gives a white solid 322 which is used in the next step without further purification.

### Step 2. 6-[2-3-chloro-pyridin-2-yl)-2H-pyrazol-3-ylmethyl]-5-propyl-[1,2,4]triazolo[4,3-a]pyrazine (323)

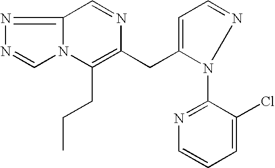

A solution of 2-[2-(3-chloro-pyridin-2-yl)-2H-pyrazol-3-ylmethyl]-5-methyl-3-propyl-pyrazine (322) in formic acid (2 mL) is heated in a sealed tube at 110° C. overnight. Excess formic acid is removed in vacuo and to the residue is added NaHCO₃ (aq.) (10 mL) and DCM (15 mL). The organic layer is separated and the aqueous layer is extracted with DCM (2×10 mL). The combined organic layers are dried (NaSO₄) and solvent removed. The crude product is separated by PTLC (5% MeOH in DCM) to give 323.

### Step 3. 2-{5-[(5-propyl[1,2,4]triazolo[4,3-a]pyrazin-6-yl)methyl]-1H-pyrazol-1-yl}nicotinonitrile (324)

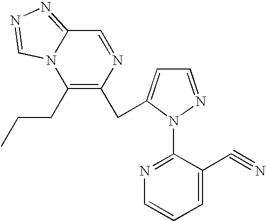

To a solution of 6-[2-3-chloro-pyridin-2-yl)-2H-pyrazol-3-ylmethyl]-5-propyl-[1,2,4]triazolo[4,3-a]pyrazine (323) (50 mg) and Zn(CN)₂ (50 mg) in DMF (15 mL), DPPF (50 mg) and Pd₂(dba)₃ (50 mg) are added. The mixture is degassed for 10 minutes and heated at 110° C. overnight. The solvent is removed in vacuo and EtOAc (10 mL) and water (10 mL) are added to the residue. The layers are separated and the aqueous layer is extracted with EtOAc (2×10 mL). The combined extracts are washed with brine (10 mL), dried (Na₂SO₄), and solvent evaporated. Column separation of the residue with 5% MeOH in DCM provides the product 324. ¹H NMR δ (CDCl₃) 1.07 (t, 3H), 1.74 (h, 2H), 3.04 (t, 2H), 4.66 (s, 2H), 6.07 (s, 1H), 7.39 (dd, 1H), 7.72 (d, 1H), 8.13 (dd, 1H), 8.62 (d, 1H), 8.89 (s, 1H), 9.16 (s, 1H).

### Example 7

### Synthesis of Pyridines

### A. 2-{5-[(5-Methoxy-3-propylpyridin-2-yl)methyl]-1H-pyrazol-1-yl}nicotinonitrile (410)

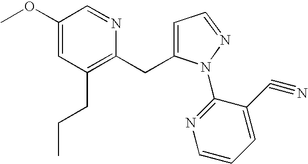

### Step 1. Pyridine-2-yl-carbamic acid tert-butyl ester (401)

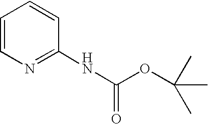

To a solution of 2-amino pyridine (47.06, 0.5 mmol) in anhydrous DCM (300 mL) at 0° C. under nitrogen is added a solution of trimethylacetyl chloride (60.29 g, 0.5 mol, 1.0 eq.) in 100 mL of DCM dropwise. The resulting mixture is stirred at 0° C. for 60 minutes, then at room temperature overnight. Water (300 mL) is added. The organic layer is collected, washed with water and brine, dried over Na₂SO₄, and concentrated. The crude product is recrystallized with 250 mL hexane at −10° C. to give the product 401.

### Step 2. (3-propyl-pyridin-2-yl)-carbamic acid tert-butyl ester (402)

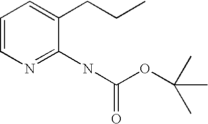

To a solution of pyridine-2-yl-carbamic acid tert-butyl ester (401) (32.26 g, 181 mmol) in anhydrous THF (200 mL) cooled to −78° C. under nitrogen, is added BuLi (1.6 M in hexane, 250 mL, 400 mmol, 2.2 eq.). The resulting solution is stirred at −78° C. for 30 minutes, gradually warmed to −10° C. and stirred for additional 2 hours. The reaction mixture is cooled to −78° C. again, and 67.7 g of iodopropane (400 mmol, 2.2 eq.) in 200 mL of anhydrous THF is added dropwise. The reaction mixture is stirred at −78° C. for 2 hours, and at room temperature overnight. The reaction is quenched with a saturated ammonium chloride solution (100 mL). Upon removal of the solvent, the residue is extracted with EtOAc (200 mL×3), washed with water and brine, and dried over Na₂SO₄. Concentration and purification through silica gel chromatography (hexanes/EtOAc, from 4:1 to 1:1) affords the product 402. ¹H NMR (400 MHz, CDCl₃) δ 8.26 (1H, dd, J=1.6, 4.8 Hz), 7.86 (1H, s, br), 7.57 (1H, dd, J=1.6, 7.6 Hz), 7.12 (1H, J=5.2, 8.0 Hz), 2.54 (2H, t, J=7.8 Hz), 1.61 (2H, m), 1.34 (9H, s), 0.92 (3H, t, J=7.2 Hz).

### Step 3. 3-Propyl-pyridin-2-ylamine (403)

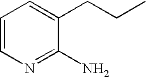

To a solution of (3-propyl-pyridin-2-yl)-carbamic acid tert-butyl ester (402) (30.56 g, 139 mmol) in EtOH (200 mL), is added 10.0N NaOH (80 mL). The reaction mixture is heated to reflux for 4 hours. Upon removal of the solvent, the residue is extracted with DCM (150 mL×3), washed with water and brine, and dried over Na₂SO₄. Concentration gives the product 403. ¹H NMR (400 MHz, CDCl₃) δ 7.94 (1H, dd, J=1.6, 4.8 Hz), 7.26 (1H, dd, J=1.6, 7.2 Hz), 6.64 (1H, J=5.2, 7.8 Hz), 4.40 (2H, s, br), 2.40 (2H, t, J=1.8 Hz), 1.65 (2H, m), 0.99 (3H, J=7.2 Hz).

### Step 4. 3-propyl-5-nitro-pyridin-2-ylamine (404)

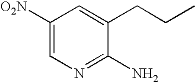

To concentrated sulfuric acid (26.0 mL) at 0° C. with vigorous stirring, 3-propyl-pyridin-2-ylamine (403) is added dropwise to keep the internal temperature lower than 20° C. over 40 minutes. The reaction mixture is cooled to −20° C., and to the mixture a solution of concentrated sulfuric acid (10 mL) and fuming nitric acid (10 mL) is added with vigorous stirring to keep the internal temperature lower than 0° C. The reaction mixture is stirred at 0° C. for 1 hour, then allowed to warm to room temperature over about 3 hours. The reaction mixture is heated to 50° C. for additional 4 hours, and then cooled to room temperature, pour onto crushed ice, and basified with 10.0N ammonium hydroxide solution. The precipitate is collected by filtration, washed with water and dried under house vacuum at 60° C. with nitrogen flow to give the title compound 404 as a dark-green solid. ¹H NMR (400 MHz, CDCl₃) δ 12.94 (2H, s, br), 8.52 (1H, s), 8.06 (1H, s), 2-56 (2H, t, J=6.8 Hz), 1.67 (2H, m), 1.02 (3H, t, J=7.2 Hz).

### Step 5. 2-Bromo-3-propyl-5-nitro-pyridine (405)

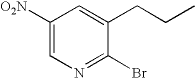

3-Propyl-5-nitro-pyridin-2-ylamine (404) (13.2 g, 72.8 mmol) is added to hydrobromic acid (48%, 41 mL, 362 mmol) in portion at 0° C. with vigorous stirring. The mixture is stirred until the internal temperature has dropped to −15 to −20° C. with an ice-salt-EtOH bath. To the mixture is added bromine (10.9 mL, 215 mmol) dropwise over 15 minutes to maintain the internal temperature below 0° C. The resulting mixture is stirred at this temperature for 90 minutes, and to the mixture a solution of sodium nitrite (17.5 g, 253 mmol) in 25 mL of water is added dropwise. The mixture is allowed to warm to room temperature over 1 hour, and stirred at this temperature for additional 1 hour. The reaction mixture is cooled to −15 to −20° C. again, and to the mixture a solution off sodium hydroxide (26.7 g, 667 mmol) in 40 mL of water is added slowly to keep the internal temperature below −10° C. The mixture is allowed to warm to room temperature, and stirred for additional 1 hour. The mixture is extracted with ether (100×3), washed with water and brine, and dried over Na₂SO₄. Concentration and purification through silica gel column flash chromatography (hexane/EtOAc, 20:1) afford the title compound 405. ¹H NMR (400 MHz, CDCl₃) δ 9.03 (1H, d, J=2.8 Hz), 8.26 (1H, d, J=2.8 Hz), 2.81 (2H, t, J=7.8 Hz), 1.7 (2H, m), 1.05 (3H, t, J=7.8 Hz).

### Step 6. 3-Nitro-6-[2-(3-bromo-pyridin-2-yl)-2H-pyrazol-3-ylmethyl]-5-propyl-pyridine (406)

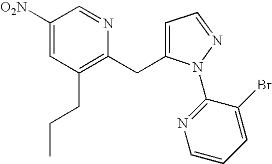

A solution of [2-(3-bromo-pyridin-2-yl)-2H-pyrazol-3-yl]-acetic acid ethyl ester (1.63 g, 6.65 mmol) in 6 mL of DMSO is added to a suspension of sodium hydride (60% in mineral oil, 293 mg, 7.32 mmol, 1.1 eq.) in 9 mL of anhydrous DMSO at 5° C. The reaction mixture is stirred at room temperature for 20 minutes, and then a solution of compound 405 (2.06 g, 6.65 mmol, 1.0 eq.) in 3 mL of DMSO is added. The resulting mixture is heated to 60° C. for 3 hours. After cooling to room temperature, 15 mL of saturated ammonium hydroxide solution is added to quench the reaction, and the mixture is diluted with 40 mL of water, extracted with EtOAc (30 mL×2), washed with water and brine, and dried over Na₂SO₄. Concentration and purification with silica gel column flash chromatography (hexane/EtOAc, 4:1) affords the title compound 406. ¹H NMR (300 MHz, CDCl₃) δ 9.04 (1H, d, J=2.4 Hz), 8.46 (1H, dd, J=1.5, 4.8 Hz), 8.16 (1H, d, J=2.4 Hz), 8-05 (1H, dd, J=1.5, 8.1 Hz), 7.66 (1H, J=1.5 Hz), 7.26 (1H, dd, J=4.5, 7.8 Hz), 6.11 (1H, d, J=15 Hz), 4.36 (2H, s), 2.59 (2H, t, J=7.5 Hz), 1.57 (2H, m), 0.95 (3H, t, J=7.5 Hz).

### Step 7. 3-Amino-6-[2-(3-bromo-pyridin-2-yl)-2H-pyrazol-3-ylmethyl]-5-propyl-pyridine (407)

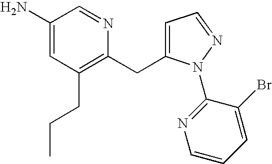

To the solution of compound 406 (570 mg, 1.42 mmol) in 20 mL of EtOAc is added tin chloride monohydrate (990 mg, 4.26 mmol, 3.0 eq.). The resulting mixture is refluxed overnight. After cooling to room temperature, 20 mL of water is added, and the mixture is basified with 1.0N NaOH, washed with brine, and dried over Na₂SO₄. Concentration affords the title compound 407.

### Step 8. 6-{[1-(3-Bromopyridin-2-yl)-1H-pyrazol-5-yl]methyl}-5-propylpyridin-3-ol (408)

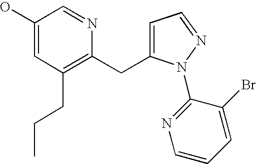

To a solution of 3-amino-6-[2-(3-bromo-pyridin-2-yl)-2H-pyrazol-3-ylmethyl]-5-propylpyridine (407) (310 mg, 0.83 mmol) in 5 mL of 5% sulfuric acid at 0° C. is added a solution of sodium nitrite (69 mg, 1.0 mmol, 1.2 eq.) in 1.0 mL of water dropwise. The resulting mixture is stirred at 0° C. for 30 minutes, and then added dropwise to a boiling 5% sulfuric acid solution (5 mL). The reaction mixture is refluxed for 10 minutes, then cooled to 0° C., basified to pH=7.0 with 10.0N NaOH, extracted with DCM, washed with water and brine, and dried over Na₂SO₄. Concentration and purification with preparative silica gel PTLC (hexane/EtOAc, 1:1) affords the title compound 408. ¹H NMR (300 MHz, CDCl₃) δ 8.47 (1H, dd, J=1.5, 4.5 Hz), 8.02 (1H, dd, J=1.8, 8.1 Hz), 7.85 (1H, d, J=2.7 Hz), 7.60 (1H, d, J=2.1 Hz), 7.24 (1H, dd, J=4.5, 8.1 Hz), 6.94 (1H, d, J=2.4 Hz), 5.95 (1H, d, J=1.5 Hz), 4.09 (2H, s), 2.36 (2H, t, J=7.2 Hz), 1.44 (2H, m), 0.86 (3H, t, J=7.2 Hz).

### Step 9. 2-{[1-(3-Bromopyridin-2-yl)-1H-pyrazol-5-yl]methyl}-5-methoxy-3-propylpyridine (409)

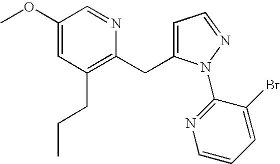

A solution of 6-{[1-(3-bromopyridin-2-yl)-1H-pyrazol-5-yl]methyl}-5-propylpyridin-3-ol (408) (60 mg, 0.16 mmol) in 2 mL of anhydrous DMF is added dropwise to a suspension of sodium hydride (60%, 7.7 mg, 0.19 mmol, 1.2 eq.). The resulting mixture is stirred at 0° C. for 10 minutes, and then 2.0 eq. of alkyl halide is added. The reaction mixture is stirred at the same temperature for 20 minutes and quenched with 2.0 mL of saturated ammonium hydroxide solution. DMF is removed, and the residue is diluted with 30 mL of DCM, washed with water and brine, and dried over Na₂SO₄. Concentration and purification with preparative silica gel TLC plate (hexane/EtOAc, 4:1) affords the title product 409 as an oil. ¹H NMR (300 MHz, CDCl₃) δ 8.50 (1H, d, J=3.0 Hz), 8.04 (1H, d, J=7.8 Hz), 7.97 (1H, d, J=2.4 Hz), 7.62 (1H, s), 7.26 (1H, m), 6.93 (1H, d, J=2.1 Hz), 6.00 (1H, s), 4.13 (2H, s), 3.81 (3H, s), 2.43 (2H, t, J=7.8 Hz), 1.48 (2H, m), 0.89 (3H, t, J=7.5 Hz).

### Step 10. 2-{5-[(5-Methoxy-3-propylpyridin-2-yl)methyl]-1H-pyrazol-1-yl}nicotinonitrile (410)

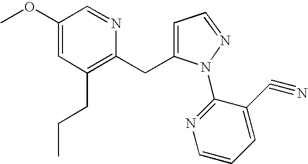

Compound 410 is synthesized via a procedure similar to that illustrated by Example 1D (step 3) using 409. ¹H NMR (300 MHz, CDCl₃) δ 8.59 (1H, dd, J=1.8, 4.8 Hz), 8.13 (1H, dd, J=1.8, 7.8 Hz), 7.99 (1H, d, J=2.7 Hz), 7.69 (1H, d, J=1.5 Hz), 7.32 (1H, dd, J=4.8, 7.5 Hz), 6.98 (1H, d, J=3.0 Hz), 5.98 (1H, d, J=1.5 Hz), 4.53 (2H, s), 3.82 (3H, s), 2.54 (2H, J=7.2 Hz), 1.58 (2H, m), 0.94 (3H, J=7.2 Hz).

Compounds 411 and 412 are synthesized via a similar procedure.

### 2-{[1-(3-bromopyridin-2-yl)-1H-pyrazol-5-yl]methyl}-5-isopropoxy-3-propylpyridine (411)

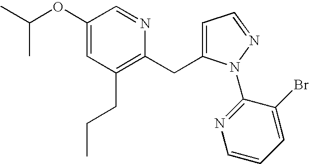

¹H NMR (300 MHz, CDCl₃) δ 8.50 (1H, dd, J=1.5, 4.5 Hz), 8.03 (1H, dd, J=1.5, 7.8 Hz), 7.93 (1H, d, J=2.7 Hz), 7.62 (1H, d, J=1.5 Hz), 7.25 (1H, dd, J=4.8, 8.1 Hz), 6.91 (1H, d, J=2.7 Hz), 6.033 (1H, d, J=1.5 Hz), 4.52 (1H, m, J=5.7 Hz), 4.13 (2H, s), 2.42 (2H, t, J=7.5 Hz), 1.48 (2H, m), 1.32 (6H, d, J=6.0 Hz), 0.89 (3H, t, J=7.5 Hz).

### 2-{5-[(5-isopropoxy-3-propylpyridin-2-yl)methyl]-1H-pyrazol-1-yl}nicotinonitrile (412)

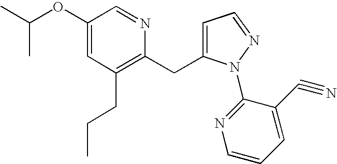

¹H NMR (300 MHz, CDCl₃) δ 8.59 (1H, dd, J=1.5, 4.5 Hz), 8.12 (1H, dd, J=1.8, 7.8 Hz), 7.96 (1H, d, J=3.0 Hz), 7.69 (1H, d, J=1.5 Hz), 7.32 (1H, dd, J=5.1, 8.1 Hz), 6.97 (1H, d, J=2.7 Hz), 6.00 (1H, s), 4.48˜4.57 (3, m, overlapped), 2.53 (2H, J=7.2 Hz), 1.58 (2H, m), 1.32 (6H, d, J=6.0 Hz), 0.94 (3H, J=7.5 Hz).

### B. 2-{5-[(5-fluoro-3-propylpyridin-2-yl)methyl]-1H-pyrazol-1-yl}nicotinonitrile (414)

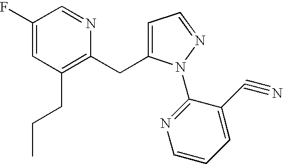

### Step 1. 2-{[1-(3-Bromopyridin-2-yl)-1H-pyrazol-5-yl]methyl}-5-fluoro-3-propylpyridine (413)

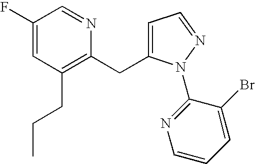

To a solution of 3-amino-6-[2-(3-bromo-pyridin-2-yl)-2H-pyrazol-3-ylmethyl]-5-propylpyridine 407 (260 mg, 0.70 mmol) in 3 mL of hydrogen fluoride-pyridine cooled to 0° C. is added sodium nitrite (60 mg, 1.3 eq.) in portions. The mixture is stirred at 0° C. for 30 minutes, and heated at 50° C. for an additional 1 hour. The reaction mixture is poured onto crushed ice, basified carefully to pH=7.0 with NaHCO₃ solution, extracted with DCM, washed with water and brine, and dried over Na₂SO₄. Concentration and purification with preparative silica gel TLC (hexane/EtOAc, 4:1) affords the title compound 413 as an oil. ¹H NMR (400 MHz, CDCl₃) δ 8.48 (1H, d, J=2.7 Hz), 8.11 (1H, d, J=2.7 Hz), 8.04 (1H, d, J=7.8 Hz), 7.63 (1H, d, J=1.2 Hz), 7.24 (1H, dd, J=4.5, 8.1 Hz), 7.12 (1H, dd, J=2.4, 9.0 Hz), 6.04 (1H, s), 4.19 (2H, s), 2.46 (2H, t, J=7.8 Hz), 1.50 (2H, m), 0.90 (3H, t, J=7.5 Hz).

### Step 2. 2-{5-[(5-Fluoro-3-propylpyridin-2-yl)methyl]-1H-pyrazol-1-yl}nicotinonitrile (414)

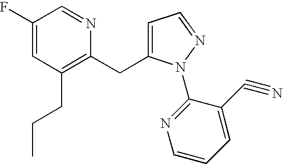

Compound 414 is synthesized via a procedure similar to that illustrated by Example 1D (step 3) using 413. ¹H NMR (400 MHz, CDCl₃) δ 8.55 (1H, dd, J=1.8, 4.5 Hz), 8.14 (2H, m, overlapped), 7.71 (1H, d, J=1.5 Hz), 7.32 (1H, dd, J=1.8, 7.8 Hz), 7.19 (1H, dd, J=2.7, 9.0 Hz), 6.02 (1H, d, J=1.5 Hz), 4.58 (2H, s), 2.59 (2H, t, J=7.8 Hz), 1.60 (2H, m), 0.96 (3H, t, J=7.5 Hz).

### Example 8

### Ligand Binding Assay

A. Purified Rat Cortical Membranes

Purified rat cortical membranes are prepared according to Procedure 1 or Procedure 2:

Procedure 1: Frozen rat cortex is homogenized in ice cold 50 mM Tris 7.4 (1 g cortex/150 mL buffer) using a POLYTRON homogenizer (setting 5 for 30 seconds). The suspension is poured into centrifuge tubes, and then centrifuged for 15 minutes at 20,000 rpm in a SS34 rotor (48,000×g). The supernatants are discarded and the pellets are washed twice with same buffer and centrifuge speed. The final pellets are stored in covered centrifuge tubes at −80° C. Prior to use, the washed rat cortical membrane is thawed and re-suspended in ice cold 50 mM Tris 7.4 (6.7 mg frozen cortex weight/mL buffer).

Procedure 2: Rat cortical tissue is dissected and homogenized in 25 volumes (w/v) of Buffer A (0.05 M Tris HCl buffer, pH 7.4 at 4° C.). The tissue homogenate is centrifuged in the cold (4° C.) at 20,000×g for 20 minutes. The supernatant is decanted, the pellet rehomogenized in the same volume of buffer, and centrifuged again at 20,000×g. The supernatant of this centrifugation step is decanted and the pellet stored at −20° C. overnight. The pellet is then thawed and resuspended in 25 volumes of Buffer A (original wt/vol), centrifuged at 20,000×g and the supernatant decanted. This wash step is repeated once. The pellet is finally resuspended in 50 volumes of Buffer A.

B. Radioligand Binding Assays

The affinity of compounds provided herein for the benzodiazepine site of the GABA_{A} receptor is confirmed using a binding assay essentially described by Thomas and Tallman (J. Bio. Chem. (1981) 156:9838-9842 and J. Neurosci. (1983) 3:433-440). Membranes prepared via Procedure 1 are assayed according to Method 1, and membranes prepared via Procedure 2 are assayed according to Method 2.

Method 1: Incubations are carried out at 1.2 mg membrane/well. Duplicate samples containing 180 μL of membrane suspension, 20 μL of ³H-Ro15-1788 (3H-Flumazenil (PerkinElmer Life Sciences, Boston, Mass.) and 2 μL of test compound or control in DMSO (total volume of 202 μL) are incubated at 4° C. for 60 minutes. The incubation is terminated by rapid filtration through untreated 102×258 mm filter mats on Tomtec filtration manifold (Hamden, Conn.) and the filters are rinsed three times with ice cold 50 mM Tris 7.4. The filters are air dried and counted on a Wallac 1205 Betaplate Liquid Scintillation Counter. Nonspecific binding (control) is determined by displacement of ³H-RO15-1788 by 10⁻⁶ M 4-oxo-4,5,6,7-tetrahydro-1H-indole-3-carboxylic acid [4-(2-propylamino-ethoxy)-phenyl]-amide. Percent inhibition of total specific binding (Total Specific Binding=Total−Nonspecific) is calculated for each compound.

Method 2: Incubations contain 100 μl of tissue homogenate, 100 μl of radioligand (0.5 nM ³H-RO15-1788, specific activity 80 Ci/mmol) and test compound or control (see below), and are brought to a total volume of 500 μl with Buffer A. Incubations are carried out for 30 minutes at 4° C. and then rapidly filtered through Whatman GFB filters to separate free and bound ligand. Filters are washed twice with fresh Buffer A and counted in a liquid scintillation counter. Nonspecific binding (control) is determined by displacement of ³H RO15-1788 with 10 μm Diazepam (Research Biochemicals International, Natick, Mass.). Data are collected in triplicate, averaged, and percent inhibition of total specific binding (Total Specific Binding=Total−Nonspecific) is calculated for each compound.

Analysis: A competition binding curve is obtained with up to 11 points (e.g., 7 points) spanning the test compound concentration range from 10^{-12 m} or 10⁻¹¹ M to 10^{-5 m}. IC₅₀ and Hill coefficient (“nH”) are determined by fitting the displacement binding data with the aid of SIGMAPLOT software (SPSS Inc., Chicago, Ill.). The Kᵢ is calculated using the Cheng-Prusoff equation (Biochemical Pharmacology 22:3099-3108 (1973)): Kᵢ=IC₅₀/(1+[L]/K_{d}), where IC₅₀ is determined as by SIGMAPLOT as the concentration of compound which displaces ½ the maximal ³H-Ro15-1788 binding, [L] is the ³H-Ro15-1788 concentration used to label the target, and K_{d} is the binding dissociation constant of ³H-Ro15-1788, previously determined to be 1.0 mM. Preferred compounds exhibit Kᵢ values of less than 100 nM and more preferred compounds exhibit Kᵢ values of less than 10 nM.

### Example 9

### Electrophysiology

The following assay is used to determine if a compound alters the electrical properties of a cell and if it acts as an agonist, an antagonist or an inverse agonist at the benzodiazepine site of the GABA_{A} receptor.

Assays are carried out essentially as described in White and Gurley (1995) NeuroReport 6:1313-16 and White et al. (1995) Receptors and Channels 3:1-5, with modifications. Electrophysiological recordings are carried out using the two electrode voltage-clamp technique at a membrane holding potential of −70 mV. Xenopus laevis oocytes are enzymatically isolated and injected with non-polyadenylated cRNA mixed in a ratio of 4:1:4 for α, β and γ subunits, respectively. Of the nine combinations of α, β and γ subunits described in the White et al. publications, preferred combinations are α₁β₂γ₂, α₂β₃γ₂, α₃β₃γ₂ and α₅β₃γ₂. Preferably all of the subunit cRNAs in each combination are human clones or all are rat clones. Each of these cloned subunits is described in GENBANK, e.g., human α₁, GENBANK accession no. X14766, human α₂, GENBANK accession no. A28100; human α₃, GENBANK accession no. A28102; human α₅, GENBANK accession no. A28104; human β₂, GENBANK accession no. M82919; human β₃, GENBANK accession no. Z20136; human γ₂, GENBANK accession no. X15376; rat α₁, GENBANK accession no. L08490, rat α₂, GENBANK accession no. L08491; rat β₃, GENBANK accession no. L08492; rat α₅, GENBANK accession no. L08494; rat β₂, GENBANK accession no. X15467; rat β₃, GENBANK accession no. X15468; and rat γ₂, GENBANK accession no. L08497. For each subunit combination, sufficient message for each constituent subunit is injected to provide current amplitudes of >10 nA when 1 μM GABA is applied.

Compounds are evaluated against a GABA concentration that evokes <10% of the maximal evocable GABA current (e.g., 1 μM-9 μM). Each oocyte is exposed to increasing concentrations of a compound being evaluated (test compound) in order to evaluate a concentration/effect relationship. Test compound efficacy is calculated as a percent-change in current amplitude: 100*((Ic/I)−1), where Ic is the GABA evoked current amplitude observed in the presence of test compound and I is the GABA evoked current amplitude observed in the absence of the test compound.

Specificity of a test compound for the benzodiazepine site is determined following completion of a concentration/effect curve. After washing the oocyte sufficiently to remove previously applied test compound, the oocyte is exposed to GABA+1 μM RO15-1788, followed by exposure to GABA+1 μM RO15-1788+test compound. Percent change due to addition of compound is calculated as described above. Any percent change observed in the presence of RO15-1788 is subtracted from the percent changes in current amplitude observed in the absence of 1 μM RO15-1788. These net values are used for the calculation of average efficacy and EC₅₀ values by standard methods. To evaluate average efficacy and EC₅₀ values, the concentration/effect data are averaged across cells and fit to the logistic equation.

### Example 10

### MDCK Toxicity Assay

This Example illustrates the evaluation of compound toxicity using a Madin Darby canine kidney (MDCK) cell cytotoxicity assay.

1 μL of test compound is added to each well of a clear bottom 96-well plate (PACKARD, Meriden, Conn.) to give final concentration of compound in the assay of 10 micromolar, 100 micromolar or 200 micromolar. Solvent without test compound is added to control wells.

MDCK cells, ATCC no. CCL-34 (American Type Culture Collection, Manassas, Va.), are maintained in sterile conditions following the instructions in the ATCC production information sheet. Confluent MDCK cells are trypsinized, harvested and diluted to a concentration of 0.1×10⁶ cells/mL with warm (37° C.) medium (VITACELL Minimum Essential Medium Eagle, ATCC catalog #30-2003). 100 μL of diluted cells is added to each well, except for five standard curve control wells that contain 100 μL of warm medium without cells. The plate is then incubated at 37° C. under 95% O₂, 5% CO₂ for 2 hours with constant shaking. After incubation, 50 μL of mammalian cell lysis solution is added per well, the wells are covered with PACKARD TOPSEAL stickers, and plates are shaken at approximately 700 rpm on a suitable shaker for 2 minutes.

Compounds causing toxicity will decrease ATP production, relative to untreated cells. The PACKARD, (Meriden, Conn.) ATP-LITE-M Luminescent ATP detection kit, product no. 6016941, is generally used according to the manufacturer's instructions to measure ATP production in treated and untreated MDCK cells. PACKARD ATP LITE-M reagents are allowed to equilibrate to room temperature. Once equilibrated, the lyophilized substrate solution is reconstituted in 5.5 mL of substrate buffer solution (from kit). Lyophilized ATP standard solution is reconstituted in deionized water to give a 10 mM stock. For the five control wells, 10 μL of serially diluted PACKARD standard is added to each of the standard curve control wells to yield a final concentration in each subsequent well of 200 nM, 100 nM, 50 nM, 25 nM and 12.5 nM. PACKARD substrate solution (50 μL) is added to all wells, which are then covered, and the plates are shaken at approximately 700 rpm on a suitable shaker for 2 minutes. A white PACKARD sticker is attached to the bottom of each plate and samples are dark adapted by wrapping plates in foil and placing in the dark for 10 minutes. Luminescence is then measured at 22° C. using a luminescence counter (e.g., PACKARD TOPCOUNT Microplate Scintillation and Luminescence Counter or TECAN SPECTRAFLUOR PLUS), and ATP levels calculated from the standard curve. ATP levels in cells treated with test compound(s) are compared to the levels determined for untreated cells. Cells treated with 10 μM of a preferred test compound exhibit ATP levels that are at least 80%, preferably at least 90%, of the untreated cells. When a 100 μM concentration of the test compound is used, cells treated with preferred test compounds exhibit ATP levels that are at least 50%, preferably at least 80%, of the ATP levels detected in untreated cells.

1. A compound of the formula: 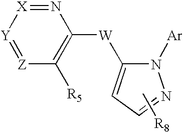
or a pharmaceutically acceptable salt thereof, wherein:
W is CR₆R₇ or O;
X is nitrogen, NO or CR₁;
Y is nitrogen, NO or CR₂;
Z is nitrogen, NO or CR₃; such that no more than two of X, Y and Z are nitrogen or NO;
R₁ is chosen from R_{C};
With respect to R₂ and R₃:
(i) R₂ and R₃ are independently chosen from R_{C}; or
(ii) Z is nitrogen and R₂ is taken together with Z to form a fused, 5-membered heteroaryl that contains 2 or 3 ring nitrogen atoms, with remaining ring atoms being carbon, and that is substituted with from 0 to 3 substituents independently chosen from R₄; or
(iii) X is nitrogen and R₂ is taken together with X to form a fused, 5-membered heteroaryl that contains 1, 2 or 3 ring nitrogen atoms, with remaining ring atoms being carbon, and that is substituted with from 0 to 3 substituents independently chosen from R₄; and R₃ is chosen from R_{C}; or
(iv) Y is nitrogen and R₃ is taken together with Y to form a fused 5-membered heteroaryl that contains 2 or 3 ring nitrogen atoms, with remaining ring atoms being carbon, and that is substituted with from 0 to 3 substituents independently chosen from R₄;
Each R₄ is independently chosen from R_{C};
R₅ is:
(a) hydrogen, halogen or cyano; or
(b) C₁-C₆alkyl, C₂-C₆alkenyl, C₂-C₆alkynyl, C₁-C₄alkoxy, or mono- or di-(C₁-C₄alkyl)amino, each of which is substituted with from 0 to 5 substituents independently chosen from halogen, hydroxy, nitro, cyano, amino, C₁-C₄alkoxy, C₁-C₂haloalkyl, C₁-C₂haloalkoxy, mono- and di-(C₁-C₄alkyl)amino, C₃-C₈cycloalkyl, phenyl, phenylC₁-C₄alkoxy and 5- or 6-membered heteroaryl;
such that if W is O, then R₅ is not hydrogen;
R₆ and R₇ are independently hydrogen, methyl, ethyl or halogen;
R₈ represents 0, 1 or 2 substituents independently chosen from halogen, hydroxy, nitro, cyano, amino, C₁-C₄alkyl, C₁-C₄alkoxy, mono- and di(C₁-C₄alkyl)amino, C₃-C₇cycloalkyl, C₁-C₂haloalkyl and C₁-C₂haloalkoxy;
Each R_{C} is independently chosen from:
(a) hydrogen, halogen, nitro and cyano; and
(b) groups of the formula: 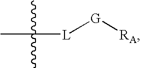
wherein:
L is absent, a single covalent bond or C₁-C₈alkylene;
G is a single covalent bond, N(R_{B}), O, C(═O), C(═O)O, C(═O)N(R_{B}), N(R_{B})C(═O), S(O)ₘ, CH₂C(═O), S(O)ₘN(R_{B}) or N(R_{B})S(O)ₘ; wherein m is 0, 1 or 2; and
R_{A} and each R_{B} are independently selected from:
(i) hydrogen; and
(ii) C₁-C₈alkyl, C₂-C₈alkenyl, C₂-C₈alkynyl, (C₃-C₈cycloalkyl)C₀-C₄alkyl, (3- to 7-membered heterocycloalkyl)C₀-C₄alkyl, (C₆-C₁₀aryl)C₀-C₂alkyl and (5- to 10-membered heteroaryl)C₀-C₂alkyl, each of which is substituted with from 0 to 4 substituents independently selected from halogen, hydroxy, nitro, cyano, amino, oxo, C₁-C₄alkyl, C₁-C₄alkoxy, C₁-C₄alkanoyl, mono- and di(C₁-C₄alkyl)amino, C₁-C₄haloalkyl and C₁-C₄haloalkoxy; and
Ar represents phenyl, naphthyl or 5- to 10-membered heteroaryl, each of which is substituted with from 0 to 4 substituents independently chosen from halogen, hydroxy, nitro, cyano, amino, aminocarbonyl, C₁-C₈alkyl, C₂-C₈alkenyl, C₂-C₈alkynyl, C₁-C₈alkoxy, (C₃-C₇cycloalkyl)C₀-C₄alkyl, (C₃-C₇cycloalkyl)C₁-C₄alkoxy, C₂-C₈alkyl ether, C₁-C₈alkanone, C₁-C₈alkanoyl, (3- to 7-membered heterocycle)C₀-C₄alkyl, C₁-C₈haloalkyl, C₁-C₈haloalkoxy, oxo, C₁-C₈hydroxyalkyl, C₁-C₈aminoalkyl, and mono- and di(C₁-C₈alkyl)aminoC₀-C₈alkyl.

2. A compound or salt according to claim 1, wherein R₈ represents 0 substituents or 1 substituent selected from halogen, C₁-C₂alkyl and C₁-C₂alkoxy.

3. A compound or salt according to claim 1, wherein Ar is substituted with 0, 1, 2 or 3 substituents independently selected from halogen, hydroxy, amino, cyano, aminocarbonyl, C₁-C₄alkyl, C₁-C₄alkoxy, mono- or di-(C₁-C₄alkyl)amino, C₂-C₄alkanoyl, (C₃-C₇cycloalkyl)C₀-C₂alkyl, C₁-C₄aminoalkyl, C₁-C₄haloalkyl, C₁-C₄haloalkoxy and 5-membered heteroaryl.

4. A compound or salt according to claim 1, wherein Ar represents phenyl, pyridyl, thiazolyl, thienyl, pyridazinyl or pyrimidinyl, each of which is substituted with from 0 to 3 substituents.

5. A compound or salt according to claim 4, wherein Ar represents phenyl, pyridyl, thiazolyl, thienyl or pyridazinyl, each of which is substituted with from 0 to 2 substituents independently selected from halogen, hydroxy, cyano, amino, aminocarbonyl, C₁-C₄alkyl, C₁-C₄-aminoalkyl, C₁-C₄alkoxy, mono- or di-(C₁-C₂alkyl)amino, C₁-C₂haloalkyl, C₁-C₂haloalkoxy and 5-membered heteroaryl.

6. A compound or salt according to claim 5, wherein Ar represents phenyl, pyridin-2-yl, 1,3-thiazol-2-yl, thien-2-yl or pyridazin-3-yl, each of which is substituted with from 0 to 3 substituents independently selected from fluoro, chloro, bromo, hydroxy, aminocarbonyl, thiazolyl, aminomethyl, methyl, ethyl, cyano, methoxy and ethoxy.

7. A compound or salt according to claim 5, wherein Ar represents 3-cyano-phenyl, pyridin-2-yl, 3-fluoro-pyridin-2-yl, 3-bromo-pyridin-2-yl, 3-chloro-pyridin-2-yl, 3-cyano-pyridin-2-yl, 3-aminomethyl-pyridin-2-yl, 3-aminocarbonyl-pyridin-2-yl, 3-thiazolyl-pyridin-2-yl, 6-fluoro-pyridin-2-yl or 6-cyano-pyridin-2-yl.

8. A compound or salt according to claim 1, wherein R₁, R₂ and R₃ are independently selected from:
(a) hydrogen, halogen, nitro or cyano; and
(b) groups of the formula: 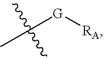
wherein:
(i) G is a single covalent bond, NH, N(R_{B}), O, C(═O)O or C(═O); and
(ii) R_{A} and R_{B} are independently selected from (1) hydrogen and (2) C₁-C₆alkyl, C₂-C₆alkenyl, (C₃-C₇cycloalkyl)C₀-C₄alkyl, (3- to 7-membered heterocycloalkyl)C₀-C₄alkyl, phenylC₀-C₄alkyl and (5- or 6-membered heteroaryl)C₀-C₄alkyl, each of which is substituted with from 0 to 4 substituents independently selected from hydroxy, halogen, cyano, amino, C₁-C₂alkyl and C₁-C₂alkoxy.

9. A compound or salt according to claim 8 wherein R₁, R₂ and R₃ are independently selected from hydrogen, hydroxy, halogen, cyano, amino, aminocarbonyl, nitro, C₁-C₆alkyl, C₂-C₆alkenyl, C₁-C₆alkoxy, C₂-C₆alkyl ether, C₃-C₇cycloalkylC₀-C₄alkyl, C₃-C₇cycloalkylC₁-C₄alkoxy, C₁-C₄hydroxyalkyl, C₁-C₆haloalkyl, C₁-C₆haloalkoxy, mono- or di(C₁-C₆alkyl)amino C₁-C₆alkanoyl, C₁-C₆alkoxycarbonyl, mono- and di-(C₁-C₄alkyl)amino, phenylC₀-C₄alkyl, phenylC₁-C₄alkoxy, thienyl, pyridyl, pyrimidinyl, thiazolyl and pyrazinyl.

10. A compound or salt according to claim 9, wherein R₁ is hydrogen, methyl or ethyl.

11. A compound or salt according to claim 8, wherein the compound has the formula:

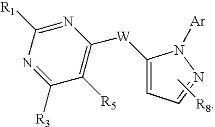

12. A compound or salt according to claim 8, wherein the compound has the formula:

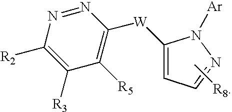

13. A compound or salt according to claim 8, wherein the compound has the formula:

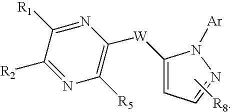

14. A compound or salt according to claim 8, wherein the compound has the formula:

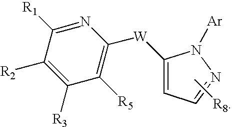

15. A compound or salt according to claim 1, wherein the compound has the formula: 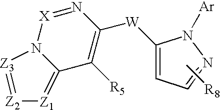
wherein Z₁, Z₂ and Z₃ are independently nitrogen or CR₄ such that exactly one or two of Z₁, Z₂ and Z₃ are nitrogen.

16. A compound or salt according to claim 15, wherein Z₁ and Z₃ are nitrogen and Z₂ is CR₄.

17. A compound or salt according to claim 15, wherein Z₁ is nitrogen and Z₂ and Z₃ are independently chosen from CR₄.

18. A compound or salt according to claim 15, wherein Z₁ is CR₄, Z₂ is nitrogen and Z₃ is CR₄.

19. A compound or salt according to claim 15, wherein each R₄ is independently:
(a) hydrogen, halogen, cyano, amino or aminocarbonyl; or
(b) C₁-C₄alkyl, C₁-C₄haloalkyl, C₁-C₄hydroxyalkyl, C₁-C₄alkoxy, C₁-C₄alkoxycarbonyl, C₂-C₄alkyl ether, (C₃-C₇cycloalkyl)C₀-C₂alkyl, (3- to 7-membered heterocycle)C₀-C₂alkyl, mono- or di-(C₁-C₄alkyl)aminocarbonyl or phenyl, each of which is substituted with from 0 to 2 substituents independently chosen from halogen, methyl and ethyl.

20. A compound or salt according to claim 1, wherein the compound has the formula: 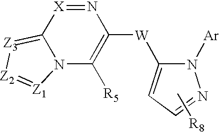
wherein:
Z₁, Z₂ and Z₃ are independently nitrogen or CR₄ such that exactly one or two of Z₁, Z₂ and Z₃ are nitrogen.

21-22. (canceled)

23. A compound or salt according to claim 1, wherein each R₄ is independently hydrogen, chloro, fluoro, cyano, amino, aminocarbonyl, methyl, ethyl, isopropyl, t-butyl, cyclopentylmethyl, methoxymethyl, ethoxymethyl, ethoxyethyl, hydroxymethyl, aminomethyl, methylaminocarbonyl, mono-, di- or tri-fluoromethyl, or (4- to 6-membered heterocycle)C₀-C₁alkyl that is optionally substituted with one or two substituents independently chosen from fluoro, chloro, methyl and ethyl.

24. A compound or salt according to claim 1, wherein W is CR₆R₇, and wherein R₆ and R₇ are both hydrogen.

25. A compound or salt according to claim 1, wherein W is O.

26. A compound or salt according to claim 1, wherein R₅ is C₁-C₆alkyl, C₂-C₆alkenyl, C₁-C₄alkoxy, or mono- or di-C₁-C₄alkylamino, each of which is substituted with from 0 to 3 substituents independently selected from halogen, hydroxy, C₁-C₂alkoxy, C₃-C₈cycloalkyl, phenyl and phenylC₁-C₂alkoxy.

27. A compound or salt according to claim 26, wherein R₅ is ethyl, propyl, butyl, ethoxy or methoxymethyl.

28. A compound or salt according to claim 1, wherein the compound exhibits a Kᵢ of 1 micromolar or less in an assay of GABA_{A} receptor binding.

29. A pharmaceutical composition comprising a compound or salt according to claim 1 in combination with a physiologically acceptable carrier or excipient.

30. A pharmaceutical composition according to claim 29, wherein the pharmaceutical composition is formulated as an injectable fluid, an aerosol, a cream, a gel, a pill, a capsule, a syrup or a transdermal patch.

31. A method for the treatment of anxiety, depression, sleepwalking, a sleep disorder, attention deficit disorder or Alzheimer's dementia, comprising administering to a patient in need of such treatment a therapeutically effective amount of a compound or salt according to claim 1.

32-39. (canceled)

40. A packaged pharmaceutical preparation comprising a pharmaceutical composition according to claim 29 in a container and instructions for using the composition to treat a patient suffering from anxiety, depression, sleepwalking, a sleep disorder, attention deficit disorder, Alzheimer's dementia or short-term memory loss.

41. (canceled)
